SEQUENCE LISTING (30 sequences)

SEQ ID NO: 1 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAVYAFYGMWPLLLLLLALPQRAYALDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRQAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

SEQ ID NO: 2 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTGPASAYQVRNSTGLYHVTNDCPNSSIVFEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHISGHRMAWDMMMNWSPTAALLVAQLLRIPQAILDMIAGAHWGVLAGMAYFSMVGNWAKVLVVLLLFAGVDAETYVTGGSAARTTAGLASLFSPGAKQNIQLVNTNGSWHINSTALNCNDSLNTGWIAGLFYHHKFNSSGCSERLASCRPLTDFAQGWGPISHADGSGPDQRPYCWHYPPKPCGIVPAKSVCGPVYCFTPSPVVVGTTDRSGAPTYSWGANDTDVFVLNNTRPPLGNWFGCTWMNSTGFTKVCGAPPCVIGGVGNNTLRCPTDCFRKHPEATYSRCGSGPWITPRCLVDYPYRLWHYPCTINYTVFKVRMYVGGVEHRLEAACNWTRGDRCNLDDRDRSELSPLLLSTTQWQVLPCSFTTLPALSTGLIHLHQNIVDVQYLYGVGSSIASWAIKWEYVVLLFLLLADARVCSCLWMMLLISQVEAALENLVVLNAASLAGTHGLVSFLVFFCFAWYLKGKWVPGAVYALYGMWPLLLLLLALPQRAYALDTEVAASCGGVVLVGLMALTLSPYYKRYISWCLWWLQYFLTRIEAQLHVWIPPLNVRGGRDAVILLMCVVHPALVFDITKLLLAAFGPLWILQASLLKVPYFVRVQGLLRICALARKMAGGHYVQMAIIKLGALTGTYVYNHLTPLRDWAHNGLRDLAVAVEPVVFSRMETKLITWGADTAACGDIINGLPVSARRGREILLGPADGMVSKGWRLLAPITAYAQQTRGLLGCIITSLTGRDKNQVEGEIQIVSTAAQTFLATCINGVCWTVYHGAGTRTIASPKGPVIQMYTNVDKDLVGWPAPQGARSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVGHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSITVPHPNIEEVALSTTGEIPFYGKAIPLEAIKGGRHLIFCHSKKKCDELAAKLVALGINAVAYYRGLDVSVIPTSGDVVVVATDALMTGYTGDFDSVIDCNTCVTQTVDLSLDPTFTIETTTLPQDAVSRTQRRGRTGRGKPGIYRFVALGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLTRLKPTLHGPTPLLYRLGAVQNEVTLTHPITKYIMTCMSADLEVVTSTWVLVGGVLAALASYCLSTGCVVIVGRIVLSGKPAIIPDREVLYREFDEMEECSQHLPYIEQGMMLAEQFKQKALGLLQTASRQAEVVAPAVQTNWQKLEAFWAKHMWNFISGIHYLAGLSTLPGNPAIASLMAFTAAVTSPLTTSQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGTHVSPTHYVPESDAAARVTTILSSLTVTQLLRRLHQWISSECTTPCSGSWLRDIWDWICEVLSDFKTWLKAKLMPQLPGIPFVSCQRGYKGVWRGDGIMHTRCHCGAEITGHVKNGTMRIVGPKTCRNMWSGTFPINAYTTGPCTPLPAPNYTFALWRVSAEEYVEIRQVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSMASSSASQLSAPSLRATCTTNHDSPDAELIEANLLWRQEMGGNITRVESENKVVILDSFDPLVAEEDEREVSVPAEILRKSRKFTPALPIWARPDYNPPLVEPWKKPDYEPPVVHGCPLPPPQSPPVPPPRKKRTVILTESTLPTALAELATKSFGSSSTSGITGDDTTTSPEPASSSCPPDSDAESYSSMPPLEGEPGDPDLSDGSWSTVSSEADKEDVVCCSMSYTWTGALVTPCAAEEQKLPINALSNSLLRHHNLVYSTTSRSACQRQKKVTFDRLQVLDGHYQDVLKEVKAAASKVKANLLSVEEACSLTPPHSAKSKFGYGAKDVRCHARKAVNHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTEHDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGELNRVAACLRKLGVPPLRAWRHRARNVRARLLSRGGRAAICGKYLFNWAVRTKLKLTPIAAAGRLDLSGWFTAGYSGGGIFHSVSRARPRWFWSCLLLLAAGVGIYLLPNR

SEQ ID NO: 3 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTGPASAYQVRNSTGLYHVTNDCPNSSIVFEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHISGHRMAWDMMMNWSPTAALLVAQLLRIPQAILDMIAGAHWGVLAGMAYFSMVGNWAKVLVVLLLFAGVDAETYVTGGSAARTTAGLASLFSPGAKQNIQLVNTNGSWHINSTALNCNDSLNTGWIAGLFYHHKFNSSGCSERLASCRPLTDFAQGWGPISHADGSGPDQRPYCWHYPPKPCGIVPAKSVCGPVYCFTPSPVVVGTTDRSGAPTYSWGANDTDVFVLNNTRPPLGNWFGCTWMNSTGFTKVCGAPPCVIGGVGNNTLRCPTDCFRKHPEATYSRCGSGPWITPRCLVDYPYRLWHYPCTINYTVFKVRMYVGGVEHRLEAACNWTRGDRCNLDDRDRSELSPLLLSTTQWQVLPCSFTTLPALSTGLIHLHQNIVDVQYLYGVGSSIASWAIKWEYVVLLFLLLADARVCSCLWMMLLISQVEAALENLVVLNAASLAGTHGLVSFLVFFCFAWYLKGKWVPGAVYALYGMWPLLLLLLALPQRAYALDTEVAASCGGVVLVGLMALTLSPYYKRYISWCLWWLQYFLTRIEAQLHVWIPPLNVRGGRDAVILLMCVVHPALVFDITKLLLAAFGPLWILQASLLKVPYFVRVQGLLRICALARKMAGGHYVQMAIIKLGALTGTYVYNHLTPLRDWAHNGLRDLAVAVEPVVFSRMETKLITWGADTAACGDIINGLPVSARRGREILLGPADGMVSKGWRLLAPITAYAQQTRGLLGCIITSLTGRDKNQVEGEIQIVSTAAQTFLATCINGVCWTVYHGAGTRTIASPKGPVIQMYTNVDKDLVGWPAPQGARSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVGHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSITVPHPNIEEVALSTTGEIPFYGKAIPLEAIKGGRHLIFCHSKKKCDELAAKLVALGINAVAYYRGLDVSVIPTSGDVVVVATDALMTGYTGDFDSVIDCNTCVTQTVDLSLDPTFTIETTTLPQDAVSRTQRRGRTGRGKPGIYRFVALGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLTRLKPTLHGPTPLLYRLGAVQNEVTLTHPITKYIMTCMSADLEVVTSTWVLVGGVLAALASYCLSTGCVVIVGRIVLSGKPAIIPDREVLYREFDEMEECSQHLPYIEQGMMLAEQFKQKALGLLQTASRQAEVVAPAVQTNWQKLEAFWAKHMWNFISGIHYLAGLSTLPGNPAIASLMAFTAAVTSPLTTSQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGTHVSPTHYVPESDAAARVTTILSSLTVTQLLRRLHQWISSECTTPCSGSWLRDIWDWICEVLSDFKTWLKAKLMPQLPGIPFVSCQRGYKGVWRGDGIMHTRCHCGAEITGHVKNGTMRIVGPKTCRNMWSGTFPINAYTTGPCTPLPAPNYTFALWRVSAEEYVEIRQVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSMASSSASQLSAPSLRATCTTNHDSPDAELIEANLLWRQEMGGNITRVESENKVVILDSFDPLVAEEDEREVSVPAEILRKSRKFTPALPIWARPDYNPPLVEPWKKPDYEPPVVHGCPLPPPQSPPVPPPRKKRTVILTESTLPTALAELATKSFGSSSASGITGDDTTTSPEPASSGCPPDSDAESYSSMPPLEGEPGDPDLSDGSWSTVSSEADKEDVVRCSMSYTWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVNHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTENDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIILFAPTLWARMILMTHFFSVLIARDQLEQALDCEIYGACYSIEPLDLPPIIQRLHGLSAFSLHSYSPGEINRVAACLRKLGVPPLRAWRHRARNVRARLLSRGGRAAICGKYLFNWAVRTKLKLTPIAAAGRLDLSGWFTAGYSGGGIFHSVSRARPRWFWSCLLLLAAGVGIYLLPNR

SEQ ID NO: 4 - protein, 3011 aa, Hepatitis C virus
MSTNPKPQRKNKRNTNRRPQDVKFPGGGQIVSGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTGPASAYQVRNSTGLYHVTNDCPNSSIVFEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHISGHRMAWDMMMNWSPTAALLVAQLLRIPQAILDMIAGAHWGVLAGMAYFSMAGNWAKVLVVLLLFAGVDAETYVTGGSAARTTAGLASLLSPGAKQKIQLVNTNGSWHINSTALNCNDSLNTGWIAGLFYHHKFNSSGCSERLASCRPLTDFAQGWGPISHADGSGPDQRPYCWHYPPKPCGIVPAKSVCGPVYCFTPSPVVVGTTDRSGAPTYSWGANDTDVFVLNNTRPPLGNWFGCTWMNSTGFTKVCGAPPCVIGGVGNNTLRCPTDCFRKHPEATYSRCGSGPWITPRCLVDYPYRLWHYPCTINYTVFKVRMYVGGVEHRLEAACNWTRGDRCNLDDRDRSELSPLLLSTTQWQVLPCSFTTLPALSTGLIHLHQNIVDVQYLYGVGSSIASWAIKWEYIVLLFLLLADARVCSCLWMMLLISQVEAALENLVVLNAASLAGTHGLVSFLVFFCFAWYLKGKWVPGAAYALYGMWPLLLLLLALPQRAYALDTEVAASCGGVVLVGLMALTLSPYYKRYISWCLWWLQYFLTRIEAQLHAWIPPLNVRGGRDAVILLMCVVHPALVFDITKLLLAAFGPLWILQASLLKVPYFVRVQGLLRICALARKMAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEIQIVSTAAQTFLATCINGVCWTVYHGAGTRTIASPKGPVIQMYTNVDKDLVGWPAPQGARSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVGHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGVRLVVLATATPPGSITVPHPNIEEVALSTTGEIPFYGKAIPLEAIKGGRHLIFCHSKKKCDELAAKLVALGINAVAYYRGLDVSVIPTSGDVVVVATDALMTGYTGDFDSVIDCNTSVTQTVDLSLDPTFTIETTTLPQDAVSRTQRRGRTGRGKPGIYRFVALGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLTRLKPTLHGPTPLLYRLGAVQNEVTLTHPITKYIMTCMSADLEVVTSTWVLVGGVLAALASYCLSTGCVVIVGRIVLSGKPAIIPDREVLYREFDEMEECSQHLPYIEQGMMLAEQFKQKALGLLQTASRQAEVVAPAVQTNWQKLEAFWAKHMWNFISGIHYLAGLSTLPGNPAIASLMAFTAAVTSPLTTSQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPAEGAVQWMNRLIAFASRGTHVSPTHYVPESDAAARVTTILSSLTVTQLLRRLHQWISSECTTPCSGSWLRDIWDWICEVLSDFKTWLKAKLMPQLPGIPFVSCQRGYKGVWRGDGIMHTRCHCGAEITGHVKNGTMRIVGPKTCRNMWSGTFPINAYTTGPCTPLPAPNYTFALWRVSAEEYVEIRQVGDFHYVTGMTTDNLKCPCQVPSPEFFTELDGVRIHRFAPPCKPLLREEVSFRVGLHEYPVGSQLPCEPEPDVAVLTSMLTDPSHITAEAAGRRLARGSPPSMASSSASQLSAPSLRATCTTNHDSPDAELIEANLLWRQEMGGNITRVESENKVVILDSFDPLVAEEDEREVSVPAEILRKSRKFTPALPIWARPDYNPPLVEPWKKPDYEPPVVHGCPLPPPQSPPVPPPRKKRTVILTESTLPTALAELATKSFGSSSTSGITGDDTTTSPEPASSSCPPDSDAESYSSMPPLEGEPGDPDLSDGSWSTVSSEADKEDAVCCSMSYTWTGALVTPCAAEEQKLPINALSNSLLRHHNLVYSTTSRSACQRQKKVTFDRLQVLDGHYQDVLKEVKAAASKVKANLLSVEEACSLTPPHSAKSKFGYGAKDVRCHARKAVNHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTEHDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGELNRVAACLRKLGVPPLRAWRHRARNVRARLLSRGGRAAICGKYLFNWAVRTKLKLTPIAAAGRLDLSGWFTAGYSGGGIFHSVSRARPRWFWSCLLLLAAGVGIYLLPNR

SEQ ID NO: 5 - protein, 3033 aa, Hepatitis C virus
MSTNPKPQRKTKRNTNRRPQDVKFPGGGQIVGGVYLLPRRGPRLGVRATRKTSERSQPRGRRQPIPKDRRSTGKSWGEPGYPWPLYGNEGLGWAGWLLSPRGSRPSWGPNDPRHRSRNVGKVIDTLTCGFADLMGYIPVLGAPLGGVARALAHGVRVLEDGVNFATGNLPGCSFSIFLLALLSCITTPVSAAEVKNISTGYMVTNDCTNDSITWQLQAAVLHVPGCVPCEKVGNASQCWIPVSPNVAVQRPGALTQGLRTHIDMVVMSATLCSALYVGDLCGGVMLAAQMFIVSPQHHWFVQDCNCSIYPGTITGHRMAWDMMMNWSPSATMILAYAMRVPEVIIDIISGAHWGVMFGLAYFSMQGAWAKVVVILLLAAGVDARTHTVGGSAAQTTGRLTSLFDMGPRQKIQLVNTNGSWHINRTALNCNDSLNTGFIASLFYTHSFNSSGCPERMSACRSIEAFRVGWGALQYEDNVTNPEDMRPYCWHYPPRQCGVVSAKTVCGPVYCFTPSPVVVGTTDRLGAPTYTWGENETDVFLLNSTRPPLGSWFGCTWMNSSGYTKTCGAPPCRTRTDFNASTDLLCPTDCFRKHPDTTYLKCGSGPWLTPRCLIDYPYRLWHYPCTVNYTIFKIRMYVGGVEHRLTAACNFTRGDRCNLEDRDRSQLSPLLHSTTEWAILPCSYSDLPALSTGLLHLHQNIVDVQFMYGLSPALTKYIVRWEWVILLFLLLADARVCACLWMLILLGQAEAALEKLVILHTASAASCNGFLYFAIFSVAAWYIKGRVVPLATYSLTGLWSFSLLLLALPQQAYAYDASVHGQIGAALLAMITLFTLTPGYKTLLSRFLWWLCYLLTLGEAMVQEWAPPMQVRGGRDGIIWAVAIFYPGVVFDITKWLLAVLGPAYLLKGALTRVPYFVRAHALLRMCTMARHLAGGRYVQMALLALGRWTGTYIYDHLTPMSDWAASGLRDLAVAVEPIIFSPMEKKVIVWGAETAACGDILHGLPVSARLGREVLLGPADGYTSKGWSLLAPITAYAQQTRGLLGTIVVSMTGRDKTEQAGEIQVLSTVTQSFLGTSISGVLWTVYHGAGNKTLAGSRGLVTQMYSSAEGDLVGWPSPPGTKSLEPCTCGAVDLYLVTRNADVIPVRRRGDKRGALLSPRPLSTLKGSSGGPVLCPRGHAVGVFRAAVCSRGVAKSIDFIPVETLDIVTRSPTFSDNSTPPAVPQTYQVGYLHAPTGSGKSTKVPVAYAAQGYKVLVLNPSVAATLGFGAYLSKAHGINPNIRTGVRTVTTGAPITYSTYGKFLADGGCAGGAYDIIICDECHAVDSTTVLGIGTVLDQAETAGVRLTVLATATPPGSVTTPHPNIEEVALGQEGEIPFYGRAIPLSYIKGGRHLIFCHSKKKCDELAAALRGMGLNSVAYYRGLDVSVIPTQGDVVVVATDALMTGYTGDFDSVIDCNVAVTQVVDLSLDPTFTITTQIVPQDAVSRSQRRGRTGRGRPGIYRYVSTGERASGMFDSVVLCECYDAGAAWYELTPSETTVRLRAYFNTPGLPVCQDHLEFWEAVFTGLTHIDAHFLSQTKQSGENFAYLTAYQATVCARAKAPPPSWDVMWKCLTRLKPTLVGPTPLLYRLGSVTNEVTLTHPVTKYIATCMQADLEVMTSTWVLAGGVLAAVASYCLATGCVCIIGRLHINQRAVVAPDKEVLYEAFDEMEECASRAALIEEGQRIAEMLKSKIQGLLQQASKQAQDIQPTVQASWPKVEQFWAKHMWNFISGIQYLAGLSTLPGNPAVASMMAFSAALTSPLSTSTTILLNILGGWLASQIAPPAGATGFVISGLVGAAVGSIGLGKVLVDILAGYGAGISGALVAFKIMSGEKPSMEDVVNLLPGILSPGALVVGVICAAILRRHVGPGEGAVQWMNRLIAFASRGTHVAPTHYVTESDASQRVTQLLGSLTITSLLRRLHNWITEDCPIPCGGSWLRDVWDWVCTILTDFKNWLASKLFPKMPGLPFVSCQKGYKGVWAGTGIMTTRCPCGANISGNVRLGSMRITGPKTCMNIWQGTFPINCYTEGQCVPKPAPNFKVAIWRVAASEYAEVTQHGSYHYITGLTTDNLKVPCQLPSPEFFSWVDGVQIHRFAPTPKPFFRDEVSFCVGLNSFVVGSQLPCDPEPDTDVLMSMLTDPSHITAETAARRLARGSPPSEASSSASQLSAPSLRATCTTHGKAYDVDMVDANLFMGGDVTRIESGSKVVVLDSLDPMVEERSDLEPSIPSEYMLPKKRFPPALPAWARPDYNPPLVESWKRPDYQPATVAGCALPPPRKTPTPPPRRRRTVGLSEDSIGDALQQLAIKSFGQPPPSGDSGPSTGPGAADSGSQTPPDELALSETGSISSMPPLEGELGDPDLEPEQVEPQPPPQGGVAAPGSDSGSWSTCSEEDDSVVCCSMSYSWTGALITPCSPEEEKLPINPLSNSLLRYHNKVYCTTTKSASLRAKKVTFDRMQVLDSYYDSVLKDIKLAASKVTARLLTMEEACQLTPPHSARSKYGFGAKEVRSLSGRAVNHIKSVWKDLLEDSETPIPTTIMAKNEVFCVDPTKGGKKAARLIVYPDLGVRVCEKMALYDITQKLPQAVMGASYGFQYSPAQRVEFLLKAWAEKKDPMGFSYDTRCFDSTVTERDIRTEESIYRACSLPEEAHTAIHSLTERLYVGGPMFNSKGQTCGYRRCRASGVLTTSMGNTITCYVKALAACKAAGIIAPTMLVCGDDLVVISESQGTEEDERNLRAFTQAMTRYSAPPGDPPRPEYDLELITSCSSNVSVALGPQGRRRYYLTRDPTTPIARAAWETVRHSPVNSWLGNIIQYAPTIWARMVLMTHFFSILMAQDTLDQNLNFEMYGAVYSVSPLDLPAIIERLHGLDAFSLHTYTPHELTRVASALRKLGAPPLRAWKSRARAVRASLISRGGRAAVCGRYLFNWAVKTKLKLTPLPEARLLDLSSWFTVGAGGGGIYHSVSRARPRSLLFGLLLLFVGVGLFLLPAR

SEQ ID NO: 6 - protein, 3022 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGHPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCIPCVQDGNASTCWTPVTPTVAVRYVGATTASIRSHVDLLVGAATMCSALYVGDMCGAVFLVGQAFTFRPRRHQTVQTCNCSLYPGHLSGHQMAWDMMMNWSPAVGMVVAHILRLPQTLFDIIAGAHWGILAGLAYYSMQGNWAKVAIIMIMFSGVDADTHTIGGVVARRVRGLSSLLSPGPKQKLELINTNGSWHINSTALNCNDSLNTGFIAGLIYYHKFNATGCPQRLSSCKPITSFGQGWGPLAAANITGSSDDRPYCWHYAPRPCNTVSASSVCGPVYCFTPSPVVVGTTDAKGAPTYNWGANESDVFLLESLRPPSGRWFGCVWMNSTGFVKTCGAPPCDIYGGGENPRNKSDSLRCPTDCFRKHPEATYSRCGAGPWLTPRCMVDYPYRLWHYPCTVNFTLFKARMFVGGFEHRFSAACNWTRGERCNIEDRDRSEQQPLLHSTTELAILPCSFTPMPALSTGLIHLHQNIVDVQYLYGVGSGMVGWALKWEFVVLIFLLLADARVCVALWLMLMITQAEAALENLVTLNAVAAAGTHGIGWYLVAFCAAWYVRGKLVPLVTYSLTGLWSLALLVLLLPQRAYAWSGEDSATLGAGVLILFGFFTLSPWYKHWIGRLMWWNQYTICRCESALQVWVPPLLARRSRDGVILLTSLLYPSLIFDITKLLIAVLGPLYLIQAAITTTPYFVRAHVLVRLCMLVRSMMGGKYLQMIILSIGRWFNTYLYDHLAPLQHWAAAGLKDLAVATEPVIFSPMEIKIITWGADTAACGDILCGLPVSARLGREVLLGPADDYREMGWRLLAPITAYTQQTRGLLGTIVTSLTGRDKNVVTGEVQVLSTATQTFLGTTVGGVMWTVYHGAGSRTLAGVKHPALQMYTNVDQDLVGWPAPPGAKSLEPCACGSADLYLVTRDADVIPARRRGDSTASLLSPRPLACLKGSSGGPVMCPSGHVAGIFRAAVCTRGVAKALQFIPVETLSTQARSPSFSDNSTPPAVPQSYQVGYLHAPTGSGKSTKVPAAYVAQGYNVLVLNPSVAATLGFGSFMSRAYGIDPNIRTGNRTVTTGAKLTYSTYGKFLADGGCSGGAYDVIICDECHAQDATSILGIGTVLDQAETAGVRLTVLATATPPGSITVPHSNIEEVALGSEGEIPFYGKAIPIALLKGGRHLIFCHSKKKCDEIASKLRGMGLNAVAYYRGLDVSVIPTAGDVVVCATDALMTGFTGDFDSVIDCNVAVEQYVDFSLDPTFSIETRTAPQDAVSRSQRRGRTGRGRLGTYRYVTPGERPSGMFDSVVLCECYDAGCSWYDLQPAETTVRLRAYLSTPGLPVCQDHLDFWESVFTGLTHIDAHFLSQTKQQGLSFSYLTAYQATVCARAQAPPPSWDETWKCLVRLKPTLHGPTPLLYRLGPIQNEICLTHPITKYIMACMSADLEVTTSTWVLLGGVLAALAACCLSVGCVVIVGHIELGGKPALVPDKEVLYQQYDEMEECSQAAPYIEQAQVIAHQFKEKVLGLLQRATQQQAVSEPIVVTNWQKLEAFWHKHMWNFASGIQYLAGLSTLPGNPAVASLMAFTASVTSPLTTNQTVFFNILGGWVATHLAGPQSSSAFVVSGMAGAAIGGVGLGRVLLDILAGYGAGVSGALVAFKIMGGEFPTAEDMVNLLPAILSPGALVVGVICAAILRRHVGPGEGAVQWMNRLIAFASRGNHVSPTHYVPESDAAAKVTALLSSLTVTSLLRRLHQWINEDYPSPCSGDWLRDIWDWVCTVLSDFKSWLSAKIMPALPGLPFISCQKGYKGVWRGDGVMSTRCPCGATIAGHVKNGSMRLAGPRTCANMWYGTFPINEYTTGPSTPCPSPNYTRALWRVAASSYVEVRRVGDFHYITGATEDELKCPCQVPAAEFFTEVDGVRLHRYAPPCKPLLRDDITFMVGLNSYAIGSQLPCEPEPDVSVLTSMLRDPSHITAETAARRLARGSPPSEASSSASQLSAPSLKATCQTHRPHPDAELVDANLLWRQEMGSNITRVESETKVVILDSFEPLRAEIDDAELSVAAECFKKPPKYPPALPIWARPDYNPPLLDRWKAPDYEPPTVHGCALPPRGAPPVPPPRRKKTIQLDGSNVSAALAALAEKSFPSSKPQEENSSSSGVDTQSSITSKVPPSPGGESDSESCSSMPPLEGEPGDPDLSCDSWSTVSDSEEQRAVCCSMSYSWTGALITPCSAEEEKLPINPLSNSLLRHHNLVYSTSSRSASQRQKKVTFDRLQVLDDHYKTALKEVKERASKVKARMLTIEEACALVPPHSARSKFGYSAKDVRSLSSKAINQIRSVWEDLLEDTTTPIPTTIMAKNEVFCVDPAKGGRKPARLIVYPDLGVRVCEKRALYDVIQKLSIETMGSAYGFQYSPQQRVERLLKMWTSKKTPLGFSYDTRCFDSTVTEQDIRVEEEIYQCCNLEPEARKVISSLTERLYCGGPMFNSKGAQCGYRRCRASGVLPTSFGNTITCYIKATAAAKAAGLRNPDFLVCGDDLVVVAESDGVSEDRAALRAFTEAMTRYSAPPGDAPQPTYDLELITSCSSNVSVAFDGKGKRYYYLTRDATTPLARAAWETARHTPVNSWLGNIIMYAPTIWVRMVMMTHFFSILQSQEILDRPLDFEMYGDTYSVTPLDLPAIIERLHGLSAFTLHSYSPVELNRVAGTLRKLGCPPLRAWRHRARAVRAKLIAQGGKAKICGLYLFNWAVRTKTNLTPLPAAGQLDLSSWFTVGVGGNDIYHSVSRARTRHLLLYLLLLTVGVGIFLLPAR

SEQ ID NO: 7 - DNA, 9599 bp, Hepatitis C virus
  source 1..9599: mol_type = genomic DNA
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcgcgtcggcccgagggcaggacctgggctcagcccgggtacccttggcccctctatggcaatgagggttgcgggtgggcgggatggctcctgtctccccgtggctctcggcctagctggggccccacggacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttttggaagacggcgtgaactatgcaacagggaaccttcctggttgctctttctctattttccttctggccctgctctcttgtctgactggacccgcttcagcctaccaagtgcgcaactccacggggctttaccatgtcaccaatgattgccccaactcgagcattgtgttcgaggcggctgatgccatcctgcacactccggggtgtgtcccttgcgtacgcgagggtaacgcctcgaggtgttgggtggcggtaacccccacggtggccaccagggatggcaaactccccacaacgcagcttcgacgtcacatcgatctgcttgtcgggagcgccaccctctgctcggccctttacgtgggggacctgtgcgggtctgtctttcttgttggtcaactgttcaccttctctcccaggcgccactggacgacgcaagattgcaattgttctatctaccccggccatatttcaggtcaccgtatggcatgggatatgatgatgaactggtcccctacggcggcgttgttggtagctcagctgctccggatcccacaagccatcctggacatgatcgctggtgctcactggggagtcctggcgggcatggcgtatttctccatggtggggaactgggcgaaggttctggtagtgctgctgctatttgccggcgtcgacgcggaaacctacgtcaccgggggaagtgccgcccgtactacggctggacttgctagtcttttctcaccaggcgccaagcagaacatccagctggtcaacaccaacggcagttggcacatcaatagcacggccttgaactgcaatgacagcctcaacaccggctggatagcaggacttttctatcaccacaaattcaactcttcgggctgttccgagaggttagccagctgccgaccccttaccgattttgcccagggctggggccctatcagtcacgccgacggaagtggccccgaccaacgcccctactgctggcactaccctccaaaaccttgtggtattgtgcccgcaaagagcgtgtgtggcccggtatattgtttcactcccagtcccgtggtggtgggaacgaccgacaggtcgggcgcgcctacctacagctggggtgcaaatgacacggacgtcttcgtccttaacaacaccaggccaccgctgggcaattggttcggttgcacctggatgaactcaactggattcaccaaagtgtgcggagcgcccccttgcgtcatcggaggggtgggcaacaacaccttgcgctgccccactgattgtttccgcaagcatccggaagccacatactctcggtgcggctccggtccctggattacgcccaggtgcctggtcgactacccgtataggctttggcattatccttgtaccatcaactacaccgtgtttaaagtcaggatgtacgtgggaggggtcgagcacaggctggaagctgcctgcaactggacgcggggcgaccgttgtaatctggatgacagggacaggtccgagctcagcccgctgctgctgtccactacgcagtggcaggtcctcccgtgttccttcacgaccctgccagccttgtccaccggcctcatccacctccaccaaaacatcgtggacgtgcaatacttgtacggggtgggatcaagcatcgcgtcctgggccatcaagtgggaatacgtcgttctcttgttccttctgcttgcagacgcgcgcgtctgctcctgcttgtggatgatgttactcatatcccaagtggaggcggctttggagaacctcgtagtactcaatgcagcatccctggccgggacacacggtcttgtatccttcctcgtgttcttctgctttgcatggtatctgaagggtaagtgggtgcccggagcggtctacgccctctacgggatgtggcctctcctcttgctcctgttagcgttgccccagcgggcatacgcactggacacggaggtggccgcgtcgtgtggcggcgttgttcttgtcgggttaatggcgctgactctgtcaccatattacaagcgctatatcagctggtgcttgtggtggcttcagtatttcctgaccagaatagaagcgcaactgcacgtgtggattccccctctcaacgtccggggggggcgcgatgccgtcatcttactcatgtgtgttgtgcacccggctctggtatttgacatcaccaagctactgctggctgccttcgggcccctttggattcttcaagccagtttgcttaaggtaccctacttcgtgcgcgttcaaggccttctccggatctgcgcgctagcgcggaagatggctggaggccattacgtgcaaatggccatcatcaagttaggggcgcttactggcacttatgtttacaaccatctcaccccccttcgggactgggcgcacaacggcctgcgagatctggccgtggctgtggagccagtcgtcttctcccgaatggagaccaagcttatcacctggggggcagacaccgccgcgtgcggtgacatcatcaacggcttgcccgtctccgcccggaggggccgggagatactgctcgggccagccgatggaatggtctccaaggggtggagattgctggcgcccatcacggcgtacgcccagcaaacgaggggcctcctagggtgtataatcaccagtctgaccggccgggacaaaaaccaagtggagggtgagatccagattgtgtcaactgctgcccaaaccttcctggcaacgtgcatcaacggggtttgctggaccgtctaccacggggccggaacgaggaccatcgcatcacccaagggtccggttatccaaatgtataccaatgtggacaaagaccttgtgggctggcccgctcctcaaggtgcccgctcactgacaccctgtacctgcggctcctcggacctttacctggtcacgaggcacgccgatgtcattcccgtgcgccggcggggtgatagcaggggcagcctgctttcgccccggcccatttcctacttgaaaggctcctcggggggtccgctgctgtgccccgcgggacacgccgtaggcttattcagggccgcggtgtgcacccgtggagtggctaaggcggtggacttcatccctgtggagaacctagagacaaccatgaggtccccggtgttcacggacaactcctctccaccagcagtgccccagagcttccaggtgggccacctgcatgctcccaccggcagcggcaaaagcaccaaggtcccggctgcatacgcagctcagggctataaggtgctagtgctcaacccctctgtcgctgcaacactgggctttggtgcttacatgtccaaggcccatggggtcgatcctaatatcaggaccggggtgagaacaattaccactggcagccccatcacgtactccacctacggcaagttccttgccgacggcgggtgttcagggggtgcttatgacataataatttgtgacgagtgccactccacggatgccacatccatcttgggcatcggcaccgttcttgaccaagcagagaccgcgggggcgagactggttgtgctcgccaccgctacccctccgggctccatcaccgtgccccatcctaacatcgaggaggttgctctgtccactaccggagagatccctttttacggcaaggctatccccctcgaggcgatcaagggggggagacatctcatcttctgtcactcaaagaagaagtgcgacgagctcgccgcaaagctggtcgcattgggcatcaatgccgtggcctactaccgcggtcttgacgtgtctgtcatcccaaccagcggcgatgttgtcgtcgtggcgaccgatgctcttatgactggctataccggcgactttgactcggtgatagactgcaacacgtgtgtcacccagacagtcgacctcagccttgaccctaccttcaccattgagacgaccacgctcccccaggacgctgtctcccgcacacaacgccggggcaggactggcagggggaagccaggcatctacagattcgtggcactgggggagcgcccctccggcatgttcgactcgtccgttctctgtgagtgctatgacgcgggctgtgcttggtatgagctcacgcccgccgagactacagttagactacgagcgtacatgaacaccccggggctccctgtgtgccaggaccatcttgaattttgggagggcgtctttacaggcctcacccatatagatgcccatttcctatctcagacaaagcagagcggggaaaactttccttacctggtagcataccaagcaaccgtgtgcgctagggctcaagcccctcccccatcgtgggaccagatgtggaagtgtttgactcgcctcaagcccaccctccatgggccaacacccttgctatacagactgggcgctgttcagaatgaagtcaccctgacgcacccaatcaccaaatacatcatgacatgcatgtcggccgacctggaggtcgtcacgagcacctgggtgctcgtcggcggcgtcctggccgctttggcctcgtattgcctgtcaacaggctgcgtggtcatagtgggcaggattgtcctgtctgggaagccggcaattatacctgacagggaagttctctaccgggagttcgatgagatggaagagtgctctcagcacttaccgtacatcgagcaagggatgatgctcgccgagcagttcaagcagaaggccctcggcctcctgcagaccgcgtcccgtcaggcagaggttgtcgcccctgctgtccagaccaactggcaaaaactcgaggccttctgggcgaagcatatgtggaacttcatcagtgggatacactacttggcgggcttgtcaacgttgcctggtaaccccgccattgcttcattgatggcttttacagctgctgtcaccagcccactaaccactagccaaaccctcctcttcaacatactgggggggtgggtggctgcccagcttgccgcccccggtgccgccaccgcctttgtgggcgctggcttagccggcgccgcaatcggcagtgttggactggggaaggtcctcgtggacattctagcagggtatggcgcgggcgtggcgggagctcttgtagcattcaagatcatgagcggtgaggtcccctccacggaggacctagtcaacctgctgcccgccatcctctcgcctggagcccttgtagtcggtgtggtctgcgcagcaatactgcgccggcacgtcggcccgggcgagggggcagtgcaatggatgaaccggctaatcgccttcgcctcccgggggacccatgtttcccccacgcactatgtgccggagagcgatgcagctgcccgcgtcactaccatactcagcagcctcactgtaacccagctcctgaggcgactgcaccagtggataagctcggagtgtaccactccatgctccggttcctggctgagggacatctgggactggatatgcgaggtgctgagcgactttaagacctggctgaaagccaagctcatgccgcaactgcctgggattccctttgtgtcctgccagcgtgggtataagggggtctggcgaggggacggcatcatgcacactcgctgccactgtggagctgagataactggacatgtcaaaaacgggacgatgaggatcgttggtcctaagacttgcaggaacatgtggagtgggactttccccattaacgcctacaccacgggcccctgtactccccttcctgcgccgaactatacgttcgcgctgtggagggtgtctgcagaggaatacgtggagataaggcaggtgggggacttccactacgtgacgggtatgactactgacaaccttaaatgcccgtgccaggtcccatcgcccgaatttttcacagaattggacggggtgcgcctacataggtttgcgcccccttgcaagcccctgctgcgggaggaggtgtcattcagagtgggactccacgagtacccggtggggtcgcaattaccttgcgagcccgaaccggacgtggccgtgttgacgtccatgctcactgatccctcccatataacagcagaggcggccgggagaaggttggcgaggggatcacccccctctatggccagctcctcggctagccaactgtccgctccatctctcagggcaacttgcactaccaaccatgactcccctgatgctgagctcatagaggccaacctcctatggaggcaggagatgggcggcaacatcaccagggttgagtcagagaacaaagtggtgattctggactccttcgatccgcttgtggcagaggaggatgagcgggaggtctccgtgcccgcagagatactgcggaagtctcggaaattcaccccagccctacccatttgggcgcggccggactataaccccccgctggtggagccgtggaaaaagcctgactacgaaccacctgtggtccatggctgcccgcttccacctccacagtcccctcctgtgcctccacctcggaagaagcggacggtgatcctcaccgaatcaaccctacctactgccttggccgagcttgccaccaaaagttttggcagctcctcaacttccggcattacgggcgacgacacgacaacatcccctgagcccgcctcctctagctgccctcccgactccgacgctgagtcctattcttccatgccccctctggagggggagcctggggatccggatctcagcgacgggtcatggtcgacggtcagtagtgaggccgacaaggaggatgtcgtgtgctgctcaatgtcttatacctggacaggcgcactcgtcaccccgtgcgccgcggaagaacaaaaactgcccatcaacgcactaagcaactcgttgctgcgtcatcacaatctggtgtattccaccacctcacgcagtgcttgccaaaggcagaagaaagtcacatttgacagactgcaagtcctggacggccattaccaggacgtgctcaaggaggttaaggcagcggcgtcaaaagtgaaggctaacttgctatccgtagaggaagcttgcagcctgacgcccccacactcagccaaatccaagtttggctatggggcaaaagacgtccgttgccatgccagaaaggccgtaaaccacatcaactccgtgtggaaagaccttctggaagacagtgtaacaccaatagacactaccatcatggctaagaacgaggttttctgcgttcagcctgagaaggggggtcgtaagccagctcgtctcatcgtgttccccgacctgggtgtacgcgtgtgcgagaagatggccttgtacgacgtagtcagcaagctccccctagccgtgatgggaagctcctacggattccaatactcaccaggacagcgggttgaattcctcgtgcaagcgtggaagtccaagaagaccccaatggggttttcgtatgatacccgctgttttgactccacagtcactgagcatgatatccgtacggaggaggcaatctaccaatgttgtgacctggacccccaagcccgcgtggccatcaagtccctcactgagaggctttatgtcgggggccctcttaccaattcaaggggggagaactgcggctatcgcaggtgccgcgcgagcggcgtactgacgaccagctgtggtaacaccctcacctgctacatcaaggcccgagcagcctgtcgagccgcagggctccaggactgcaccatgctcgtgtgtggcgacgacttagtcgttatctgtgagagtgcgggggtccaggaggacgcggcgagcctgagagccttcacggaggctatgaccaggtactccgccccccccggggaccccccacaaccagaatacgacttggagctcataacatcatgctcctccaacgtgtcagtcgcccacgacggcgctggaaaaagggtctactacctcacccgtgaccctacaacccccctcgcgcgggccgcgtgggagacagcaagacacactccagtcaattcctggctaggcaacataattatgtttgcccccacactgtgggcgaggatgatactgatgacccatttctttagcgtcctcatagccagggatcagcttgaacaggccctcgattgcgagatctacggggcctgctactccatagaaccactggatctacctccaatcattcaaagactccatggcctcagcgcattttcactccacagttactctccaggtgaactcaatagggtggccgcatgcctcagaaaacttggggtcccgcccttgcgagcttggagacaccgggcccggaatgtccgcgctaggcttctgtccagaggaggcagggctgccatttgtggcaagtacctcttcaactgggcagtaaggacaaagctcaaactcactccaatagcggccgctggccggctagacttatctggctggttcacggctggctacagcgggggaggcatttttcacagcgtgtctcgtgcccggccccgctggttctggtcttgcctactcctgctcgctgcaggggtaggcatctacctcctccccaaccgatgaaggttggggtaaacactccggcctcttaggccatttcctgttttttttttttttttttttttttttttttttttttttttttttttctttttccctctttttcttctctttttccttctttaatggtggctccatcttagccctagtcacggctagctgtgaaaggtccgtgagccgcatgactgcagagagtgctgatactggcctctctgcagatcatgt

SEQ ID NO: 8 - DNA, 9599 bp, Hepatitis C virus
  source 1..9599: mol_type = genomic DNA
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcgcgtcggcccgagggcaggacctgggctcagcccgggtacccttggcccctctatggcaatgagggttgcgggtgggcgggatggctcctgtctccccgtggctctcggcctagctggggccccacggacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttttggaagacggcgtgaactatgcaacagggaaccttcctggttgctctttctctattttccttctggccctgctctcttgtctgactggacccgcttcagcctaccaagtgcgcaactccacggggctttaccatgtcaccaatgattgccccaactcgagcattgtgttcgaggcggctgatgccatcctgcacactccggggtgtgtcccttgcgtacgcgagggtaacgcctcgaggtgttgggtggcggtaacccccacggtggccaccagggatggcaaactccccacaacgcagcttcgacgtcacatcgatctgcttgtcgggagcgccaccctctgctcggccctttacgtgggggacctgtgcgggtctgtctttcttgttggtcaactgttcaccttctctcccaggcgccactggacgacgcaagattgcaattgttctatctaccccggccatatttcaggtcaccgtatggcatgggatatgatgatgaactggtcccctacggcggcgttgttggtagctcagctgctccggatcccacaagccatcctggacatgatcgctggtgctcactggggagtcctggcgggcatggcgtatttctccatggtggggaactgggcgaaggttctggtagtgctgctgctatttgccggcgtcgacgcggaaacctacgtcaccgggggaagtgccgcccgtactacggctggacttgctagtcttttctcaccaggcgccaagcagaacatccagctggtcaacaccaacggcagttggcacatcaatagcacggccttgaactgcaatgacagcctcaacaccggctggatagcaggacttttctatcaccacaaattcaactcttcgggctgttccgagaggttagccagctgccgaccccttaccgattttgcccagggctggggccctatcagtcacgccgacggaagtggccccgaccaacgcccctactgctggcactaccctccaaaaccttgtggtattgtgcccgcaaagagcgtgtgtggcccggtatattgtttcactcccagtcccgtggtggtgggaacgaccgacaggtcgggcgcgcctacctacagctggggtgcaaatgacacggacgtcttcgtccttaacaacaccaggccaccgctgggcaattggttcggttgcacctggatgaactcaactggattcaccaaagtgtgcggagcgcccccttgcgtcatcggaggggtgggcaacaacaccttgcgctgccccactgattgtttccgcaagcatccggaagccacatactctcggtgcggctccggtccctggattacgcccaggtgcctggtcgactacccgtataggctttggcattatccttgtaccatcaactacaccgtgtttaaagtcaggatgtacgtgggaggggtcgagcacaggctggaagctgcctgcaactggacgcggggcgaccgttgtaatctggatgacagggacaggtccgagctcagcccgctgctgctgtccactacgcagtggcaggtcctcccgtgttccttcacgaccctgccagccttgtccaccggcctcatccacctccaccaaaacatcgtggacgtgcaatacttgtacggggtgggatcaagcatcgcgtcctgggccatcaagtgggaatacgtcgttctcttgttccttctgcttgcagacgcgcgcgtctgctcctgcttgtggatgatgttactcatatcccaagtggaggcggctttggagaacctcgtagtactcaatgcagcatccctggccgggacacacggtcttgtatccttcctcgtgttcttctgctttgcatggtatctgaagggtaagtgggtgcccggagcggtctacgccctctacgggatgtggcctctcctcttgctcctgttagcgttgccccagcgggcatacgcactggacacggaggtggccgcgtcgtgtggcggcgttgttcttgtcgggttaatggcgctgactctgtcaccatattacaagcgctatatcagctggtgcttgtggtggcttcagtatttcctgaccagaatagaagcgcaactgcacgtgtggattccccctctcaacgtccggggggggcgcgatgccgtcatcttactcatgtgtgttgtgcacccggctctggtatttgacatcaccaagctactgctggctgccttcgggcccctttggattcttcaagccagtttgcttaaggtaccctacttcgtgcgcgttcaaggccttctccggatctgcgcgctagcgcggaagatggctggaggccattacgtgcaaatggccatcatcaagttaggggcgcttactggcacttatgtttacaaccatctcaccccccttcgggactgggcgcacaacggcctgcgagatctggccgtggctgtggagccagtcgtcttctcccgaatggagaccaagcttatcacctggggggcagacaccgccgcgtgcggtgacatcatcaacggcttgcccgtctccgcccggaggggccgggagatactgctcgggccagccgatggaatggtctccaaggggtggagattgctggcgcccatcacggcgtacgcccagcaaacgaggggcctcctagggtgtataatcaccagtctgaccggccgggacaaaaaccaagtggagggtgagatccagattgtgtcaactgctgcccaaaccttcctggcaacgtgcatcaacggggtttgctggaccgtctaccacggggccggaacgaggaccatcgcatcacccaagggtccggttatccaaatgtataccaatgtggacaaagaccttgtgggctggcccgctcctcaaggtgcccgctcactgacaccctgtacctgcggctcctcggacctttacctggtcacgaggcacgccgatgtcattcccgtgcgccggcggggtgatagcaggggcagcctgctttcgccccggcccatttcctacttgaaaggctcctcggggggtccgctgctgtgccccgcgggacacgccgtaggcttattcagggccgcggtgtgcacccgtggagtggctaaggcggtggacttcatccctgtggagaacctagagacaaccatgaggtccccggtgttcacggacaactcctctccaccagcagtgccccagagcttccaggtgggccacctgcatgctcccaccggcagcggcaaaagcaccaaggtcccggctgcatacgcagctcagggctataaggtgctagtgctcaacccctctgtcgctgcaacactgggctttggtgcttacatgtccaaggcccatggggtcgatcctaatatcaggaccggggtgagaacaattaccactggcagccccatcacgtactccacctacggcaagttccttgccgacggcgggtgttcagggggtgcttatgacataataatttgtgacgagtgccactccacggatgccacatccatcttgggcatcggcaccgttcttgaccaagcagagaccgcgggggcgagactggttgtgctcgccaccgctacccctccgggctccatcaccgtgccccatcctaacatcgaggaggttgctctgtccactaccggagagatccctttttacggcaaggctatccccctcgaggcgatcaagggggggagacatctcatcttctgtcactcaaagaagaagtgcgacgagctcgccgcaaagctggtcgcattgggcatcaatgccgtggcctactaccgcggtcttgacgtgtctgtcatcccaaccagcggcgatgttgtcgtcgtggcgaccgatgctcttatgactggctataccggcgactttgactcggtgatagactgcaacacgtgtgtcacccagacagtcgacctcagccttgaccctaccttcaccattgagacgaccacgctcccccaggacgctgtctcccgcacacaacgccggggcaggactggcagggggaagccaggcatctacagattcgtggcactgggggagcgcccctccggcatgttcgactcgtccgttctctgtgagtgctatgacgcgggctgtgcttggtatgagctcacgcccgccgagactacagttagactacgagcgtacatgaacaccccggggctccctgtgtgccaggaccatcttgaattttgggagggcgtctttacaggcctcacccatatagatgcccatttcctatctcagacaaagcagagcggggaaaactttccttacctggtagcataccaagcaaccgtgtgcgctagggctcaagcccctcccccatcgtgggaccagatgtggaagtgtttgactcgcctcaagcccaccctccatgggccaacacccttgctatacagactgggcgctgttcagaatgaagtcaccctgacgcacccaatcaccaaatacatcatgacatgcatgtcggccgacctggaggtcgtcacgagcacctgggtgctcgtcggcggcgtcctggccgctttggcctcgtattgcctgtcaacaggctgcgtggtcatagtgggcaggattgtcctgtctgggaagccggcaattatacctgacagggaagttctctaccgggagttcgatgagatggaagagtgctctcagcacttaccgtacatcgagcaagggatgatgctcgccgagcagttcaagcagaaggccctcggcctcctgcagaccgcgtcccgtcaggcagaggttgtcgcccctgctgtccagaccaactggcaaaaactcgaggccttctgggcgaagcatatgtggaacttcatcagtgggatacactacttggcgggcttgtcaacgttgcctggtaaccccgccattgcttcattgatggcttttacagctgctgtcaccagcccactaaccactagccaaaccctcctcttcaacatactgggggggtgggtggctgcccagcttgccgcccccggtgccgccaccgcctttgtgggcgctggcttagccggcgccgcaatcggcagtgttggactggggaaggtcctcgtggacattctagcagggtatggcgcgggcgtggcgggagctcttgtagcattcaagatcatgagcggtgaggtcccctccacggaggacctagtcaacctgctgcccgccatcctctcgcctggagcccttgtagtcggtgtggtctgcgcagcaatactgcgccggcacgtcggcccgggcgagggggcagtgcaatggatgaaccggctaatcgccttcgcctcccgggggacccatgtttcccccacgcactatgtgccggagagcgatgcagctgcccgcgtcactaccatactcagcagcctcactgtaacccagctcctgaggcgactgcaccagtggataagctcggagtgtaccactccatgctccggttcctggctgagggacatctgggactggatatgcgaggtgctgagcgactttaagacctggctgaaagccaagctcatgccgcaactgcctgggattccctttgtgtcctgccagcgtgggtataagggggtctggcgaggggacggcatcatgcacactcgctgccactgtggagctgagataactggacatgtcaaaaacgggacgatgaggatcgttggtcctaagacttgcaggaacatgtggagtgggactttccccattaacgcctacaccacgggcccctgtactccccttcctgcgccgaactatacgttcgcgctgtggagggtgtctgcagaggaatacgtggagataaggcaggtgggggacttccactacgtgacgggtatgactactgacaaccttaaatgcccgtgccaggtcccatcgcccgaatttttcacagaattggacggggtgcgcctacataggtttgcgcccccttgcaagcccctgctgcgggaggaggtgtcattcagagtgggactccacgagtacccggtggggtcgcaattaccttgcgagcccgaaccggacgtggccgtgttgacgtccatgctcactgatccctcccatataacagcagaggcggccgggagaaggttggcgaggggatcacccccctctatggccagctcctcggctagccaactgtccgctccatctctcagggcaacttgcactaccaaccatgactcccctgatgctgagctcatagaggccaacctcctatggaggcaggagatgggcggcaacatcaccagggttgagtcagagaacaaagtggtgattctggactccttcgatccgcttgtggcagaggaggatgagcgggaggtctccgtgcccgcagagatactgcggaagtctcggaaattcaccccagccctacccatttgggcgcggccggactataaccccccgctggtggagccgtggaaaaagcctgactacgaaccacctgtggtccatggctgcccgcttccacctccacagtcccctcctgtgcctccacctcggaagaagcggacggtgatcctcaccgaatcaaccctacctactgccttggccgagcttgccaccaaaagttttggcagctcctcagcttccggcattacgggcgacgacacgacaacatcccctgagcccgcctcctctggctgccctcccgactccgacgctgagtcctattcttccatgccccctctggagggggagcctggggatccggatctcagcgacgggtcatggtcgacggtcagtagtgaggccgacaaggaggatgtcgtgcgctgctcaatgtcttatacctggacaggcgcactcgtcaccccgtgcgccgcggaagaacaaaaactgcccatcaacgcactaagcaactcgttgctgcgtcatcacaatctggtgtattccaccacctcacgcagtgcttgccaaaggcagaagaaagtcacatttgacagactgcaagtcctggacagccattaccaggacgtgctcaaggaggttaaggcagcggcgtcaaaagtgaaggctaacttgctatccgtagaggaagcttgcagcctgacgcccccacactcagccaaatccaagtttggctatggggcaaaagacgtccgttgccatgccagaaaggccgtaaaccacatcaactccgtgtggaaagaccttctggaagacagtgtaacaccaatagacactaccatcatggctaagaacgaggttttctgcgttcagcctgagaaggggggtcgtaagccagctcgtctcatcgtgttccccgacctgggtgtacgcgtgtgcgagaagatggccttgtacgacgtagtcagcaagctccccctagccgtgatgggaagctcctacggattccaatactcaccaggacagcgggttgaattcctcgtgcaagcgtggaagtccaagaagaccccaatggggttttcgtatgatacccgctgttttgactccacagtcactgagaatgatatccgtacggaggaggcaatctaccaatgttgtgacctggacccccaagcccgcgtggccatcaagtccctcactgagaggctttatgtcgggggccctcttaccaattcaaggggggagaactgcggctatcgcaggtgccgcgcgagcggcgtactgacgaccagctgtggtaacaccctcacctgctacatcaaggcccgagcagcctgtcgagccgcagggctccaggactgcaccatgctcgtgtgtggcgacgacttagtcgttatctgtgagagtgcgggggtccaggaggacgcggcgagcctgagagccttcacggaggctatgaccaggtactccgccccccccggggaccccccacaaccagaatacgacttggagctcataacatcatgctcctccaacgtgtcagtcgcccacgacggcgctggaaaaagggtctactacctcacccgtgaccctacaacccccctcgcgcgggccgcgtgggagacagcaagacacactccagtcaattcctggctaggcaacataattttgtttgcccccacactgtgggcgaggatgatactgatgacccatttctttagcgtcctcatagccagggatcagcttgaacaggccctcgattgcgagatctacggggcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggaatgtccgcgctaggcttctgtccagaggaggcagggctgccatttgtggcaagtacctcttcaactgggcagtaaggacaaagctcaaactcactccaatagcggccgctggccggctagacttatctggctggttcacggctggctacagcgggggaggcatttttcacagcgtgtctcgtgcccggccccgctggttctggtcttgcctactcctgctcgctgcaggggtaggcatctacctcctccccaaccgatgaaggttggggtaaacactccggcctcttaggccatttcctgttttttttttttttttttttttttttttttttttttttttttttttctttttccctctttttcttctctttttccttctttaatggtggctccatcttagccctagtcacggctagctgtgaaaggtccgtgagccgcatgactgcagagagtgctgatactggcctctctgcagatcatgt

SEQ ID NO: 9 - DNA, 9599 bp, Hepatitis C virus
  source 1..9599: mol_type = genomic DNA
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaacaaacgtaacaccaaccgtcgcccacaggacgtcaagttcccgggtggcggtcagatcgttagtggagtttacttgttgccgcgcaggggccctagattgggtgtgcgcgcgacgaggaagacttccgagcggtcgcaacctcgaggtagacgtcagcctatccccaaggcgcgtcggcccgagggcaggacctgggctcagcccgggtacccttggcccctctatggcaatgagggttgcgggtgggcgggatggctcctgtctccccgtggctctcggcctagctggggccccacggacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttttggaagacggcgtgaactatgcaacagggaaccttcctggttgctctttctctattttccttctggccctgctctcttgtctgactggacccgcttcagcctaccaagtgcgcaactccacggggctttaccatgtcaccaatgattgccccaactcgagcattgtgttcgaggcggctgatgccatcctgcacactccggggtgtgtcccttgcgtacgcgagggtaacgcctcgaggtgttgggtggcggtaacccccacggtggccaccagggatggcaaactccccacaacgcagcttcgacgtcacatcgatctgcttgtcgggagcgccaccctctgctcggccctttacgtgggggacctgtgcgggtctgtctttcttgttggtcaactgttcaccttctctcccaggcgccactggacgacgcaagattgcaattgttctatctaccccggccatatttcaggtcaccgtatggcatgggatatgatgatgaactggtcccctacggcggcgttgttggtagctcagctgctccggatcccacaagccatcctggacatgatcgctggtgctcactggggagtcctggcgggcatggcgtatttctccatggcggggaactgggcgaaggttctggtagtgctgctgctatttgccggcgtcgacgcggaaacctacgtcaccgggggaagtgccgcccgtactacggctggacttgctagtcttctctcaccaggcgccaagcagaagatccagctggtcaacaccaacggcagttggcacatcaatagcacggccttgaactgcaatgacagcctcaacaccggctggatagcaggacttttctatcaccacaaattcaactcttcgggctgttccgagaggttagccagctgccgaccccttaccgattttgcccagggctggggccctatcagtcacgccgacggaagtggccccgaccaacgcccctactgctggcactaccctccaaaaccttgtggtattgtgcccgcaaagagcgtgtgtggcccggtatattgtttcactcccagtcccgtggtggtgggaacgaccgacaggtcgggcgcgcctacctacagctggggtgcaaatgacacggacgtcttcgtccttaacaacaccaggccaccgctgggcaattggttcggttgcacctggatgaactcaactggattcaccaaagtgtgcggagcgcccccttgcgtcatcggaggggtgggcaacaacaccttgcgctgccccactgattgtttccgcaagcatccggaagccacatactctcggtgcggctccggtccctggattacgcccaggtgcctggtcgactacccgtataggctttggcattatccttgtaccatcaactacaccgtgtttaaagtcaggatgtacgtgggaggggtcgagcacaggctggaagctgcctgcaactggacgcggggcgaccgttgtaatctggatgacagggacaggtccgagctcagcccgctgctgctgtccactacgcagtggcaggtcctcccgtgttccttcacgaccctgccagccttgtccaccggcctcatccacctccaccaaaacatcgtggacgtgcaatacttgtacggggtgggatcaagcatcgcgtcctgggccatcaagtgggaatacatcgttctcttgttccttctgcttgcagacgcgcgcgtctgctcctgcttgtggatgatgttactcatatcccaagtggaggcggctttggagaacctcgtagtactcaatgcagcatccctggccgggacacacggtcttgtatccttcctcgtgttcttctgctttgcatggtatctgaagggtaagtgggtgcccggagcggcctacgccctctacgggatgtggcctctcctcttgctcctgttagcgttgccccagcgggcatacgcactggacacggaggtggccgcgtcgtgtggcggcgttgttcttgtcgggttaatggcgctgactctgtcaccatattacaagcgctatatcagctggtgcttgtggtggcttcagtatttcctgaccagaatagaagcgcaactgcacgcgtggattccccctctcaacgtccggggggggcgcgatgccgtcatcttactcatgtgtgttgtgcacccggctctggtatttgacatcaccaagctactgctggctgccttcgggcccctttggattcttcaagccagtttgcttaaggtaccctacttcgtgcgcgttcaaggccttctccggatctgcgcgctagcgcggaagatggctggaggccattacgtgcaaatggccatcatcaagttaggggcgcttactggcacttatgtttacaaccatctcaccccccttcgggactgggcgcacaacggcctgcgagatctggccgtggctgtggagccagtcgtcttctcccgaatggagaccaagcttatcacctggggggcagacaccgccgcgtgcggtgacatcatcaacggcttgcccgtctccgcccggaggggccaggagatactgctcgggccagccgatggaatggtctccaaggggtggagattgctggcgcccatcacggcgtacgcccagcaaacgaggggcctcctagggtgtataatcaccagtctgaccggccgggacaaaaaccaagtggagggtgagatccagattgtgtcaactgctgcccaaaccttcctggcaacgtgcatcaacggggtttgctggaccgtctaccacggggccggaacgaggaccatcgcatcacccaagggtccggttatccaaatgtataccaatgtggacaaagaccttgtgggctggcccgctcctcaaggtgcccgctcactgacaccctgtacctgcggctcctcggacctttacctggtcacgaggcacgccgatgtcattcccgtgcgccggcggggtgatagcaggggcagcctgctttcgccccggcccatttcctacttgaaaggctcctcggggggtccgctgctgtgccccgcgggacacgccgtaggcttattcagggccgcggtgtgcacccgtggagtggctaaggcggtggacttcatccctgtggagaacctagagacaaccatgaggtccccggtgttcacggacaactcctctccaccagcagtgccccagagcttccaggtgggccacctgcatgctcccaccggcagcggcaaaagcaccaaggtcccggctgcatacgcagctcagggctataaggtgctagtgctcaacccctctgtcgctgcaacactgggctttggtgcttacatgtccaaggcccatggggtcgatcctaatatcaggaccggggtgagaacaattaccactggcagccccatcacgtactccacctacggcaagttccttgccgacggcgggtgttcagggggtgcttatgacataataatttgtgacgagtgccactccacggatgccacatccatcttgggcatcggcaccgttcttgaccaagcagagaccgcgggggtgagactggttgtgctcgccaccgctacccctccgggctccatcaccgtgccccatcctaacatcgaggaggttgctctgtccactaccggagagatccctttttacggcaaggctatccccctcgaggcgatcaagggggggagacatctcatcttctgtcactcaaagaagaagtgcgacgagctcgccgcaaagctggtcgcattgggcatcaatgccgtggcctactaccgcggtcttgacgtgtctgtcatcccaaccagcggcgatgttgtcgtcgtggcgaccgatgctcttatgactggctataccggcgactttgactcggtgatagactgcaacacgtctgtcacccagacagtcgacctcagccttgaccctaccttcaccattgagacgaccacgctcccccaggacgctgtctcccgcacacaacgccggggcaggactggcagggggaagccaggcatctacagattcgtggcactgggggagcgcccctccggcatgttcgactcgtccgttctctgtgagtgctatgacgcgggctgtgcttggtatgagctcacgcccgccgagactacagttagactacgagcgtacatgaacaccccggggctccctgtgtgccaggaccatcttgaattttgggagggcgtctttacaggcctcacccatatagatgcccatttcctatctcagacaaagcagagcggggaaaactttccttacctggtagcataccaagcaaccgtgtgcgctagggctcaagcccctcccccatcgtgggaccagatgtggaagtgtttgactcgcctcaagcccaccctccatgggccaacacccttgctatacagactgggcgctgttcagaatgaagtcaccctgacgcacccaatcaccaaatacatcatgacatgcatgtcggccgacctggaggtcgtcacgagcacctgggtgctcgtcggcggcgtcctggccgctttggcctcgtattgcctgtcaacaggctgcgtggtcatagtgggcaggattgtcctgtctgggaagccggcaattatacctgacagggaagttctctaccgggagttcgatgagatggaagagtgctctcagcacttaccgtacatcgagcaagggatgatgctcgccgagcagttcaagcagaaggccctcggcctcctgcagaccgcgtcccgtcaggcagaggttgtcgcccctgctgtccagaccaactggcaaaaactcgaggccttctgggcgaagcatatgtggaacttcatcagtgggatacactacttggcgggcttgtcaacgttgcctggtaaccccgccattgcttcattgatggcttttacagctgctgtcaccagcccactaaccactagccaaaccctcctcttcaacatactgggggggtgggtggctgcccagcttgccgcccccggtgccgccaccgcctttgtgggcgctggcttagccggcgccgcaatcggcagtgttggactggggaaggtcctcgtggacattctagcagggtatggcgcgggcgtggcgggagctcttgtagcattcaagatcatgagcggtgaggtcccctccacggaggacctagtcaacctgctgcccgccatcctctcgcctggagcccttgtagtcggtgtggtctgcgcagcaatactgcgccggcacgtcggcccggccgagggggcagtgcaatggatgaaccggctaatcgccttcgcctcccgggggacccatgtttcccccacgcactatgtgccggagagcgatgcagctgcccgcgtcactaccatactcagcagcctcactgtaacccagctcctgaggcgactgcaccagtggataagctcggagtgtaccactccatgctccggttcctggctgagggacatctgggactggatatgcgaggtgctgagcgactttaagacctggctgaaagccaagctcatgccgcaactgcctgggattccctttgtgtcctgccagcgtgggtataagggggtctggcgaggggacggcatcatgcacactcgctgccactgtggagctgagataactggacatgtcaaaaacgggacgatgaggatcgttggtcctaagacttgcaggaacatgtggagtgggactttccccattaacgcctacaccacgggcccctgtactccccttcctgcgccgaactatacgttcgcgctgtggagggtgtctgcagaggaatacgtggagataaggcaggtgggggacttccactacgtgacgggtatgactactgacaaccttaaatgcccgtgccaggtcccatcgcccgaatttttcacagaattggacggggtgcgcatacataggtttgcgcccccttgcaagcccctgctgcgggaggaggtgtcattcagagtgggactccacgagtacccggtggggtcgcaattaccttgcgagcccgaaccggacgtggccgtgttgacgtccatgctcactgatccctcccatataacagcagaggcggccgggagaaggttggcgaggggatcacccccctctatggccagctcctcggctagccaactgtccgctccatctctcagggcaacttgcactaccaaccatgactcccctgatgctgagctcatagaggccaacctcctatggaggcaggagatgggcggcaacatcaccagggttgagtcagagaacaaagtggtgattctggactccttcgatccgcttgtggcagaggaggatgagcgggaggtctccgtgcccgcagagatactgcggaagtctcggaaattcaccccagccctacccatttgggcgcggccggactataaccccccgctggtggagccgtggaaaaagcctgactacgaaccacctgtggtccatggctgcccgcttccacctccacagtcccctcctgtgcctccacctcggaagaagcggacggtgatcctcaccgaatcaaccctacctactgccttggccgagcttgccaccaaaagttttggcagctcctcaacttccggcattacgggcgacgacacgacaacatcccctgagcccgcctcctctagctgccctcccgactccgacgctgagtcctattcttccatgccccctctggagggggagcctggggatccggatctcagcgacgggtcatggtcgacggtcagtagtgaggccgacaaggaggatgccgtgtgctgctcaatgtcttatacctggacaggcgcactcgtcaccccgtgcgccgcggaagaacaaaaactgcccatcaacgcactaagcaactcgttgctgcgtcatcacaatctggtgtattccaccacctcacgcagtgcttgccaaaggcagaagaaagtcacatttgacagactgcaagtcctggacggccattaccaggacgtgctcaaggaggttaaggcagcggcgtcaaaagtgaaggctaacttgctatccgtagaggaagcttgcagcctgacgcccccacactcagccaaatccaagtttggctatggggcaaaagacgtccgttgccatgccagaaaggccgtaaaccacatcaactccgtgtggaaagaccttctggaagacagtgtaacaccaatagacactaccatcatggctaagaacgaggttttctgcgttcagcctgagaaggggggtcgtaagccagctcgtctcatcgtgttccccgacctgggtgtacgcgtgtgcgagaagatggccttgtacgacgtagtcagcaagctccccctagccgtgatgggaagctcctacggattccaatactcaccaggacagcgggttgaattcctcgtgcaagcgtggaagtccaagaagaccccaatggggttttcgtatgatacccgctgttttgactccacagtcactgagcatgatatccgtacggaggaggcaatctaccaatgttgtgacctggacccccaagcccgcgtggccatcaagtccctcactgagaggctttatgtcgggggccctcttaccaattcaaggggggagaactgcggctatcgcaggtgccgcgcgagcggcgtactgacgaccagctgtggtaacaccctcacctgctacatcaaggcccgagcagcctgtcgagccgcagggctccaggactgcaccatgctcgtgtgtggcgacgacttagtcgttatctgtgagagtgcgggggtccaggaggacgcggcgagcctgagagccttcacggaggctatgaccaggtactccgccccccccggggaccccccacaaccagaatacgacttggagctcataacatcatgctcctccaacgtgtcagtcgcccacgacggcgctggaaaaagggtctactacctcacccgtgaccctacaacccccctcgcgcgggccgcgtgggagacagcaagacacactccagtcaattcctggctaggcaacataattatgtttgcccccacactgtgggcgaggatgatactgatgacccatttctttagcgtcctcatagccagggatcagcttgaacaggccctcgattgcgagatctacggggcctgctactccatagaaccactggatctacctccaatcattcaaagactccatggcctcagcgcattttcactccacagttactctccaggtgaactcaatagggtggccgcatgcctcagaaaacttggggtcccgcccttgcgagcttggagacaccgggcccggaatgtccgcgctaggcttctgtccagaggaggcagggctgccatttgtggcaagtacctcttcaactgggcagtaaggacaaagctcaaactcactccaatagcggccgctggccggctagacttatctggctggttcacggctggctacagcgggggaggcatttttcacagcgtgtctcgtgcccggccccgctggttctggtcttgcctactcctgctcgctgcaggggtaggcatctacctcctccccaaccgatgaaggttggggtaaacactccggcctcttaggccatttcctgttttttttttttttttttttttttttttttttttttttttttttttctttttccctctttttcttctctttttccttctttaatggtggctccatcttagccctagtcacggctagctgtgaaaggtccgtgagccgcatgactgcagagagtgctgatactggcctctctgcagatcatgt

SEQ ID NO: 10 - DNA, 9678 bp, Hepatitis C virus
  source 1..9678: mol_type = genomic DNA
acccgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacaccaaccgtcgcccacaagacgttaagtttccgggcggcggccagatcgttggcggagtatacttgttgccgcgcaggggccccaggttgggtgtgcgcgcgacaaggaagacttcggagcggtcccagccacgtggaaggcgccagcccatccctaaagatcggcgctccactggcaaatcctggggagaaccaggatacccctggcccctatacgggaatgagggactcggctgggcaggatggctcctgtccccccgaggttcccgtccctcttggggccccaatgacccccggcataggtcgcgcaacgtgggtaaggtcatcgataccctaacgtgcggctttgccgacctcatggggtacatccctgtcctgggcgccccgctcggcggcgtcgccagagctctcgcgcatggcgtgagagtcctggaggacggggttaattttgcaacagggaacttacccggttgctccttttctatcttcttgctggccctgctgtcctgcatcaccaccccggtctccgctgccgaagtgaagaacatcagtaccggctacatggtgactaacgactgcaccaatgacagcattacctggcagctccaggctgctgtcctccacgtccccgggtgcgtcccgtgcgagaaagtggggaatgcatctcagtgctggataccggtctcaccgaatgtggccgtgcagcggcccggcgccctcacgcagggcttgcggacgcacatcgacatggttgtgatgtccgccacgctctgctctgccctctacgtgggggacctctgcggtggggtgatgctcgcagcccaaatgttcattgtctcgccgcagcaccactggtttgtccaagactgcaattgctccatctaccctggtaccatcactggacaccgcatggcatgggacatgatgatgaactggtcgccctcggctaccatgatcttggcgtacgcgatgcgtgtccccgaggtcattatagacatcattagcggggctcattggggcgtcatgttcggcttggcctacttctctatgcagggagcgtgggcgaaagtcgttgtcatccttctgttggccgccggggtggacgcgcgcacccatactgttgggggttctgccgcgcagaccaccgggcgcctcaccagcttatttgacatgggccccaggcagaaaatccagctcgttaacaccaatggcagctggcacatcaaccgcaccgccctgaactgcaatgactccttgaacaccggctttatcgcgtctctgttctacacccacagcttcaactcgtcaggatgtcccgaacgcatgtccgcctgccgcagtatcgaggccttccgggtgggatggggcgccttgcaatatgaggataatgtcaccaatccagaggatatgagaccctattgctggcactacccaccaaggcagtgtggcgtggtctccgcgaagactgtgtgtggcccagtgtactgtttcacccccagcccagtggtagtgggcacgaccgacaggcttggagcgcccacttacacgtggggggagaatgagacagatgtcttcctattgaacagcactcgaccaccgctggggtcatggttcggctgcacgtggatgaactcttctggctacaccaagacttgcggcgcaccaccctgccgtactagaactgacttcaacgccagcacggacctgttgtgccccacggactgttttaggaagcatcctgataccacttacctcaaatgcggctctgggccctggctcacgccaaggtgcctgatcgactacccctacaggctctggcattacccctgcacagttaactataccatcttcaaaataaggatgtatgtgggaggggttgagcacaggctcacggctgcatgcaatttcactcgtggggatcgttgcaacttggaggacagagacagaagtcaactgtctcctttgttgcactccaccacggaatgggccattttaccttgctcttactcggacctgcccgccttgtcgactggtcttctccacctccaccaaaacatcgtggacgtacaattcatgtatggcctatcacctgccctcacaaaatacatcgtccgatgggagtgggtaatactcttattcctgctcttagcggacgccagggtttgcgcctgcttatggatgctcatcttgttgggccaggccgaagcagcactagagaagctggtcatcttgcacactgcgagcgcagctagctgcaatggcttcctatattttgccatcttttccgtggctgcttggtacatcaagggtcgggtagtccccttagctacctattccctcactggcctgtggtcctttagcctactgctcctagcattgccccaacaggcttatgcttatgacgcatctgtgcatggccagataggagcggctctgctggcaatgatcactctctttactctcacccccgggtataagacccttctcagccggtttttgtggtggttgtgctatcttctgaccctgggggaagctatggtccaggagtgggcaccacctatgcaggtgcgcggtggccgtgatggcatcatatgggccgtcgccatattctacccaggtgtggtgtttgacataaccaagtggctcttggcggtgcttgggcctgcttacctcctaaaaggtgctttgacgcgcgtgccgtacttcgtcagggctcacgctctactgaggatgtgcaccatggcaaggcatctcgcggggggcaggtacgtccagatggcgctactagcccttggcaggtggactggcacttacatctatgaccacctcacccctatgtcggattgggctgctagtggcctgcgggacctggcggtcgccgttgagcctatcatcttcagtccgatggagaagaaagtcattgtctggggagcggagacagctgcttgtggggacattttacacggacttcccgtgtccgcccgacttggtcgggaggtcctccttggcccagctgatggctatacctccaaggggtggagtcttctcgcccccatcactgcttacgcccagcagacacgtggccttttgggcaccatagtggtgagcatgacggggcgcgacaagacagaacaggctggggaaattcaggtcctgtccacagtcactcagtccttcctcggaacatccatctcgggggttttgtggactgtctaccatggagctggcaacaagactctggccggctcacggggtctggtcacgcagatgtactccagtgctgagggggacttagtagggtggcccagcccccctgggactaaatctttggagccgtgcacgtgtggagcggtcgacctgtacctggtcacgcggaacgctgatgtcatcccggttcgaagacgcggggacaaacggggagcgctactctccccgagacctctttccaccttgaaggggtcctcaggaggcccggtgctatgccccaggggccacgctgtcggagtcttccgggcagctgtgtgctctcggggcgtggctaagtccatagatttcatccccgttgagacactcgacatcgtcacgcggtcccccacctttagtgacaacagcacaccacctgctgtgccccagacctatcaggtcgggtacttgcatgccccgactggcagtggaaagagcaccaaagttcctgtcgcatatgctgctcaggggtataaagtgctagtgcttaatccctcagtggctgccaccctggggtttggggcgtacttgtctaaggcacatggcatcaatcccaacattaggactggagtcaggactgtgacgaccggggcgcccatcacgtactccacatatggcaaattcctcgccgatgggggctgtgcgggcggcgcctacgacatcatcatatgtgatgaatgccatgccgtggactctaccaccgtccttggcatcggaacagtccttgatcaagcagagacagctggggtcagactaactgtgctggctacagctacgccccctgggtcagtgacaaccccccaccccaacatagaggaggtggcccttgggcaggagggcgagatccccttctatgggagggcgattcccctgtcttacatcaagggaggaagacatctgatcttctgccattcaaagaaaaagtgtgacgagctcgcggcggcccttcggggtatgggcttgaactcagtggcatactacagagggttggacgtctccgtaataccaactcagggagacgtagtggtcgtcgccaccgacgccctcatgacagggtatactggggactttgactccgtgatcgactgcaacgtagcggtcactcaagttgtagacctcagtttagaccccacattcaccataaccacacagattgtccctcaagacgctgtctcacgtagccagcgccggggtcgcacgggtaggggaagaccgggcatttataggtatgtttccactggtgagcgagcctcaggaatgtttgacagtgtagtgctctgtgagtgctacgacgcaggggccgcatggtatgagctcacaccatcggagaccaccgtcaggctcagggcgtatttcaacacgcccggtttgcctgtgtgccaagaccatcttgagttttgggaggcagttttcaccggcctcacacacatagatgcccacttcctttcccaaacaaagcaatcgggggaaaatttcgcatacttaacagcctaccaggctacagtgtgcgctagggccaaagccccccccccgtcctgggacgtcatgtggaagtgtttgactcgactcaagcccacactcgtgggccccacacctctcctgtaccgcttgggctctgttaccaacgaggtcaccctcacacatcccgtgacgaaatacatcgccacctgcatgcaagccgaccttgaggtcatgaccagcacatgggtcttggcagggggagtcttggcggccgtcgcctcgtattgcctggcgaccgggtgtgtttgcatcatcggccgcttgcacattaaccagcgagccgtcgttgcgccggacaaggaggtcctctatgaggcttttgatgagatggaggaatgtgcctctagggcggctctcattgaagaggggcagcggatagccgagatgctgaagtccaagatccaaggcttattgcagcaagcttccaaacaagctcaagacatacaacccactgtgcaggcttcatggcccaaggtagaacaattctgggccaaacacatgtggaacttcattagcggcatccaatacctcgcaggactatcaacactgccagggaaccctgcagtagcttccatgatggcgttcagtgccgccctcaccagtccgctgtcaacaagcaccactatccttctcaacattttggggggctggctagcatcccaaattgcaccacccgcgggggccactggcttcgttatcagtggcctagtgggagctgccgtaggcagtataggcttaggtaaggtgctagtggacatcctggcagggtatggtgcgggcatttcgggggctctcgtcgcattcaagatcatgtctggcgagaagccctccatggaggatgtcgtcaacttgctgcctggaattctgtctccgggtgccttggtagtgggagtcatctgcgcggccattctgcgccgacacgtgggaccgggggaaggcgccgtccaatggatgaatagactcattgcctttgcttccagaggaactcacgtcgcccccacccactacgtgacggagtcggatgcgtcgcagcgtgtgacccaactacttggctcccttaccataaccagcctgctcagaagactccacaactggattactgaggactgccccatcccatgcggcggctcgtggctccgcgatgtgtgggactgggtttgcaccatcctaacagactttaaaaattggctggcctccaaattattcccaaagatgcccggcctcccctttgtctcctgtcaaaaggggtacaagggcgtgtgggccggcactggcatcatgaccacacggtgtccttgcggcgccaatatctctggcaatgtccgcttgggctccatgagaatcacggggcctaagacctgcatgaatatctggcaggggacctttcctatcaattgttacacggagggccagtgcgtgccgaaacccgcgccaaactttaaggtcgccatctggagggtggcggcctcagagtacgcggaggtgacgcagcacgggtcataccactacataacaggactcaccactgataacttgaaagtcccctgccaactaccctctcccgagttcttttcctgggtggacggagtgcagatccataggtttgcccccacaccgaagccgtttttccgggatgaggtctcgttctgcgttgggcttaattcatttgtcgtcgggtcccagcttccttgcgaccctgaacccgacacagacgtattgatgtccatgctaacagatccatctcatatcacggcggagactgcagcgcggcgtttagcgcgggggtcacccccatccgaggcaagctcctcggcgagccagctatcggcaccatcgctgcgagccacctgcaccacccacggcaaagcctatgatgtggacatggtggatgctaacctgttcatggggggcgatgtgactcggatagagtctgggtccaaagtggtcgttctggactctctcgacccaatggtcgaagaaaggagcgaccttgagccttcgataccatcagaatacatgctccccaagaagaggttcccaccagctttaccggcctgggcacggcctgattacaacccaccgcttgtggaatcgtggaaaaggccagattaccaaccggccactgttgcgggctgtgctctccctcctcctaggaaaaccccgacgcctcccccaaggaggcgccggacagtgggcctaagtgaggactccataggagatgcccttcaacagctggccattaagtcctttggccagccccccccaagcggcgattcaggcccttccacggggccgggcgctgccgattccggcagtcagacgcctcctgatgagttggccctttcggagacaggttccatctcttccatgccccccctcgagggggagcttggagatccagacctggagcctgagcaggtagagccccaaccccccccccaggggggggtggcagctcccggctcggactcggggtcctggtctacttgctccgaggaggacgactccgtcgtgtgctgctccatgtcatactcctggaccggggctctaataactccttgtagtcccgaagaggagaagttaccgattaaccccttgagcaactccctgttgcgatatcacaacaaggtgtactgtaccacaacaaagagcgcctcactaagggctaaaaaggtaacttttgataggatgcaagtgctcgactcctactacgactcagtcttaaaggacattaagctagcggcctccaaggtcaccgcaaggctcctcaccatggaggaggcttgccagttaaccccaccccattctgcaagatctaaatatgggtttggggctaaggaggtccgcagcttgtccgggagggccgttaaccacatcaagtccgtgtggaaggacctcctggaggactcagaaacaccaattcccacaaccattatggccaaaaatgaggtgttctgcgtggaccccaccaaggggggcaagaaagcagctcgccttatcgtttaccctgacctcggcgtcagggtctgcgagaagatggccctttatgacattacacaaaaacttcctcaggcggtgatgggggcttcttatggattccagtattcccccgctcagcgggtagagtttctcttgaaagcatgggcggaaaagaaggaccctatgggtttttcgtatgatacccgatgctttgactcaaccgtcactgagagagacatcaggactgaggagtccatatatcgggcctgctccttgcccgaggaggcccacactgccatacactcgctaactgagagactttacgtgggagggcctatgttcaacagcaagggccaaacctgcgggtacaggcgttgccgcgccagcggggtgctcaccactagcatggggaacaccatcacatgctacgtgaaagccttagcggcttgtaaagctgcagggataatcgcgcccacaatgctggtatgcggcgatgacttggttgtcatctcagaaagccaggggaccgaggaggacgagcggaacctgagagccttcacgcaggctatgaccaggtattctgcccctcctggtgacccccccagaccggagtatgatctggagctgataacatcttgctcctcaaatgtgtctgtggcgctgggcccacaaggccgccgcagatactacctgaccagagaccctaccactccaatcgcccgggctgcctgggaaacagttagacactcccctgtcaattcatggctgggaaacatcatccagtacgccccgaccatatgggctcgcatggtcctgatgacacacttcttctccattctcatggctcaagacacgctggaccagaacctcaactttgagatgtacggagcggtgtactccgtgagtcccttggacctcccagctataattgaaaggttacatgggcttgacgctttttctctgcacacatacactccccacgaactgacacgggtggcttcagccctcagaaaacttggggcgccacccctcagagcgtggaagagccgggcacgtgcagtcagggcgtccctcatctcccgtggggggagagcggccgtttgcggtcgatatctcttcaattgggcggtgaagaccaagctcaaactcactccattgccggaagcgcgcctcctggatttatccagctggttcaccgtcggcgccggcgggggcggcatttatcacagcgtgtcgcgtgcccgaccccgctcattgctctttggcctactcctactttttgtaggggtaggccttttcctactccccgctcggtagagcggcacacattagctacactccatagctaactgtccctttttttttttttttttttttttttttttttttttttttttttttttttttctttttttctcttttccttctttcttaccttattttactttctttcctggtggctccatcttagccctagtcacggctagctgtgaaaggtccgtgagccgcatgactgcagagagtgccgtaactggtctctctgcagatcatgt

SEQ ID NO: 11 - DNA, 9629 bp, Hepatitis C virus
  source 1..9629: mol_type = genomic DNA
gcctgcctcttacgaggcgacactccaccatggatcactcccctgtgaggaacttctgtcttcacgcggaaagcgcctagccatggcgttagtacgagtgtcgtgcagcctccaggaccccccctcccgggagagccatagtggtctgcggaaccggtgagtacaccggaatcgctggggtgaccgggtcctttcttggagcaacccgctcaatacccagaaatttgggcgtgcccccgcgagatcactagccgagtagtgttgggtcgcgaaaggccttgtggtactgcctgatagggtgcttgcgagtgccccgggaggtctcgtagaccgtgcaacatgagcacacttcctaaacctcaaagaaaaaccaaaagaaacaccatccgccgcccacaggacgtcaagttcccgggtggcggacagatcgttggtggagtatacgtgttgccgcgcaggggcccacgattgggtgtgcgcgcgacgcgtaaaacttctgaacggtcacagcctcgcggacgacgacagcctatccccaaggcgcgtcggagcgaaggccggtcctgggctcagcccgggcacccctggcccctctatggtaacgagggctgcgggtgggcagggtggctcctgtccccacgcggttcccgcccatcttggggcccgaacgacccccggcgaaggtcccgcaatttgggtaaggtcatcgataccctcacgtgtggattcgccgacctcatggggtacatcccgctcgtcggcgctcccgtagggggcgtcgcaagggcccttgcgcatggcgtgagggctcttgaagacgggataaacttcgcaacagggaatttgcccggttgctccttttctatcttcctccttgctctgttctcttgcttaatccatccagcagccagtttagagtggaggaacacgtctggtctctatgtcctcactaacgactgttccaatagcagcattgtgtatgaggccgatgacgttatcctgcacacacccggctgtataccttgtgttcaggacggcaatgcatctacgtgctggaccccagtgacacctacagtggcagtcaggtacgtcggagcaaccaccgcttcgatacgcagtcacgtggacctattagtgggcgcggccacgatgtgctctgcgctctacgtaggtgatatgtgtggggccgtcttcctcgtgggacaagccttcacgttcagacctcgtcgccatcagacggtccagacctgcaactgctcgctgtacccaggccatctttcaggacaccaaatggcttgggatatgatgatgaattggtcccccgctgtgggtatggtggtggcgcacatcctgcggttgccccagaccttgttcgacataatagccggagctcattggggcatcttggcgggcctggcctattactccatgcagggcaactgggccaaggtcgctatcatcatgattatgttttcgggggtcgatgccgacacacacaccattggtggtgtcgtagctcgtagagtcagaggactttcttctttgctcagtccgggccccaagcagaaactggagctgatcaacaccaacggatcgtggcacatcaatagtactgccctgaactgcaatgattccctaaacaccgggttcatagccgggctgatctattatcataagtttaacgccactggatgtcctcaaaggctcagcagctgcaagcccatcacttccttcgggcaggggtggggccccttggcagctgctaacatcaccggctcttctgatgacagaccgtactgctggcactacgcacctagaccttgtaacacagtctcggcatcaagtgtctgcggccctgtgtactgcttcacgccgtcaccggtggtcgtgggcactactgatgctaagggcgcgccaacctacaactggggtgcgaatgagtcagacgtgttcctgctggaatccctgcggcctcccagtggtcggtggtttgggtgcgtgtggatgaactccacgggatttgtcaagacgtgcggagcccctccttgtgatatctatgggggtggggaaaatccccggaataagtcagactccctccgctgccccaccgactgcttcaggaaacatcctgaggccacatacagccggtgtggcgcggggccctggttgacacctcggtgcatggtagactacccataccggctttggcattacccatgtacagtcaattttacattgttcaaggcgaggatgtttgtaggtgggtttgagcaccggttctccgccgcctgcaactggaccaggggggagcgctgcaatatcgaggatcgtgaccgcagcgaacaacaaccgctgctgcattcaacaactgagcttgctatactgccttgctctttcacgcccatgcctgcgttgtcaacaggtctgatacacctccaccaaaacatcgtggatgtccaatacctttatggcgttggatctggcatggtgggatgggcgttgaagtgggagttcgtagtcctcattttcctcctcctagcagacgcacgcgtgtgcgttgccctttggctgatgctgatgataacgcaagcggaagcagccctggagaaccttgtcacgctgaatgctgtcgctgctgctgggacacatggtatcggctggtacctggtagccttttgcgcagcgtggtacgtgcggggtaaacttgtcccgctagtgacctacagcctgacgggactttggtccctggcactactcgttctcctgctcccccaacgggcgtatgcttggtcgggtgaggacagcgctactctcggtgctggggtcttgatcctcttcggcttctttaccttatcaccttggtacaagcattggatcggccgcctcatgtggtggaaccagtacaccatatgcaggtgcgagtccgcccttcaagtgtgggtcccccccctacttgcacgcaggagcagggacggtgtcatcctgctaacaagcctgctttatccatctttaatttttgacatcactaagctgctgatagcggtattgggcccattatacttgatacaggctgccatcactactaccccctattttgtgcgtgcgcatgtactggttcgcctttgcatgctcgtgcgctccatgatggggggaaaatacctccagatgatcatactgagcataggcaggtggttcaacacctacttatacgaccacctagcgccattgcaacactgggccgcagccggtctcaaagacctagcagtggccactgagcctgtaatattcagtcccatggaaatcaagatcatcacctggggcgcggacacagcagcttgtggagatattctttgcgggctgcccgtctctgcgcgattaggccgtgaggtattattgggacctgctgatgactatcgggagatgggttggcgcctgttggccccgatcacagcatacacccagcaaactaggggccttcttgggactattgtgaccagcttgactggcagggataagaacgtggtgaccggtgaagtgcaggtgctttctacggctacccagaccttcctaggtacaacagtagggggggttatgtggactgtttaccatggtgcaggctcgagaacactcgcgggcgtcaaacatcctgcgctccaaatgtacacaaatgtggatcaggaccttgttgggtggccagcccctccgggggctaagtctctcgaaccgtgcgcctgcggatctgcagacttatacttggttacccgcgatgctgatgtcatccctgctcggcgcaggggagactccacagcgagcttgctcagtcctaggcctcttgcctgccttaagggttcttctggaggtcctgttatgtgcccgtcggggcatgttgcggggatatttagggctgctgtgtgcaccagaggcgtagcaaaagccctacagttcataccagtggaaacccttagcacacaggctaggtctccatctttctctgacaattcaactcctcctgccgtcccacagagctatcaagtcgggtaccttcatgccccgaccggtagtggtaagagcacaaaggtcccggccgcttatgtagcgcaaggatataatgttcttgtgttgaatccatcggtggcggccacactaggcttcggctctttcatgtcgcgtgcctatgggatcgaccctaatatccgcactgggaaccgtaccgttacaactggtgctaaactgacctattccacctatggtaagtttcttgcggacgggggttgctccgggggagcatatgatgtgatcatctgtgacgagtgtcatgcccaagacgctactagcatattgggtataggcacggtcttagatcaggctgagacggctggggtgaggctgacggttctagcgacagcaactcccccaggcagcatcactgtgccacattctaacatcgaagaggtggccctaggctctgaaggtgagatccctttctacggtaaggctataccgatagccctgcttaagggggggagacaccttatcttttgccattccaagaaaaaatgtgatgagatagcatccaaactcagaggcatggggctcaacgctgtagcgtactataggggtctcgatgtgtccgtcataccaacagcaggagacgtcgtagtttgcgctactgacgccctcatgactgggttcaccggagacttcgattctgtcatagattgcaacgtagctgttgaacagtacgttgacttcagcctggaccccactttttccattgagactcgcactgctccccaagacgcggtttcccgcagccagcgtcgtggccgtacgggccgaggtagactcggcacgtaccgatatgtcacccccggtgaaagaccgtctggaatgtttgactcggttgttctctgtgagtgctatgacgcgggctgctcgtggtacgatctgcagcccgctgagaccacagttagactgagagcttacttgtccacgccggggttacctgtctgccaagaccatttggacttctgggagagcgtcttcactggactaacacacatagatgcccattttctgtcacagactaagcagcagggacttagcttctcgtacctgactgcctaccaagccactgtgtgcgcccgcgcgcaggctcctcccccaagttgggacgagacgtggaagtgtctcgtgcggcttaagccaacactacatggacctacgccccttctgtatcggttggggcctatccaaaatgaaatctgtttgacacatcccatcacaaaatacatcatggcatgcatgtcagctgatctggaagtgaccaccagcacctgggtgctgcttggaggggtccttgcggccctagcggcttgctgcttgtcagtcggctgcgttgtgattgtgggccatatcgagctggggggcaagccagcacttgttccagacaaagaggtgttgtatcaacaatacgatgagatggaggagtgctcgcaagccgccccatacatcgaacaagctcaggtaatagcccaccagttcaaggaaaaagtccttggattgctgcagcgagccacccaacaacaagctgtcagtgagcccatagtagttaccaactggcaaaagcttgaggccttctggcataagcatatgtggaactttgcgagtgggatccagtacctggcaggcctctccaccttgcctggcaaccctgctgtggcgtctcttatggcgttcactgcttcagtcaccagccccctgacgaccaaccaaactgtgttttttaacatactcggggggtgggttgctacccatttggcagggccccagagctcttctgcattcgtggtaagtggcatggccggcgctgccatagggggtgtaggcctgggcagggtcttgcttgacatcctggcaggatacggagctggtgtctcaggtgccttggtggcttttaagatcatgggaggagaattccccactgctgaagacatggtcaacctgttgcccgccatactatctccgggtgctctcgtcgtcggtgtgatatgcgctgccatactacgtcgccacgtaggacctggagagggggcggtacagtggatgaacagactcattgcgttcgcatcccggggcaaccacgtctcaccaacgcactatgtccccgagagcgatgctgcagcaaaggttaccgcattgctcagttctctaactgtcacgagtctgctccggcggttgcaccagtggatcaatgaggactacccaagtccttgcagcggcgattggttgcgtgacatctgggactgggtttgtacagtgttgagtgactttaaatcatggctctctgctaagattatgccagcgctccctggactgcctttcatttcctgtcaaaagggatataagggcgtgtggcggggggacggtgtgatgtcaacgcgctgtccttgcggagcaacaatagccggccatgtgaagaatgggtccatgcggcttgcggggccgcgtacatgtgctaacatgtggtacggtactttccccatcaatgagtacaccaccggacccagcacaccttgcccatcacccaactacactcgcgcactatggcgcgtggctgctagcagctacgtcgaggtgcgccgggtgggggacttccattatattacgggggccacagaagatgagctcaagtgtccgtgccaagtgccggctgctgagttctttactgaagtggatggggtaagactccaccgttacgcccctccatgtaagcccctgttgagagatgatatcactttcatggtggggttgaattcctacgctatagggtcccaactcccctgtgagcccgaaccagacgtttctgtgctgacctcgatgttgagagacccctctcatatcaccgccgagacggcagcgcgccgccttgcgcgcggatcccctccatcagaggcgagctcatccgccagccaattgtcggctccgtcgttgaaggccacttgccagacgcataggcctcatccagacgctgagctagtggatgccaacttgttatggcggcaagagatgggtagcaacatcacacgggtagagtctgagacgaaggttgtgattcttgattcattcgaacctctgagagccgaaattgatgacgctgagctctcggtggctgcagagtgtttcaagaaacctcccaagtatcctccagcccttcccatctgggctaggccagactataaccctccactgctcgaccgctggaaagcaccagattatgaaccaccaactgtccatggatgtgccttaccaccgcgaggcgctccaccggtgcctccccctcggaggaagaaaacaattcagctggatggttccaatgtgtccgcggcgctagctgcgctggcggaaaaatcatttccatcctcgaaaccacaggaagagaatagctcatcctctggggtcgacacacagtccagcattacttccaaggtgcccccttctccgggaggggagtccgactcagagtcgtgttcgtctatgcctcctctcgagggagagccgggcgatccagacttgagttgtgactcctggtccaccgttagtgacagcgaggagcagagagcggtctgctgctctatgtcatactcctggaccggcgccctaataacaccatgtagtgctgaggaggagaaactgcccatcaacccactcagcaattccttgttgagacatcataacctagtctattcaacgtcgtctcgaagcgcttctcagcggcagaagaaggttaccttcgacaggctgcaggtgctcgacgaccattacaagactgcattaaaggaggtaaaggagcgggcgtccaaggtaaaggcccgcatgcttaccatcgaggaagcgtgcgcgctcgtccctcctcactctgcccggtcgaagttcgggtatagtgcgaaggacgtccgctccttgtccagcaaagccattaaccagatccgctccgtctgggaggacttgctggaagacaccacaaccccaattccaactaccataatggcgaagaacgaggtattctgtgtggaccccgctaaggggggccgcaagcccgctcgcctcattgtgtaccctgacctgggggtgcgtgtctgtgagaaacgtgccctatatgacgtgatacagaagttgtcaattgagacgatggggtctgcctacggattccaatactcgcctcaacagcgggtcgaacgtctgctgaagatgtggacctcaaagaagacccccttggggttctcgtatgacacccgctgctttgactcgactgtcactgaacaggacatcagggtggaagaggagatataccaatgctgtaatcttgaaccggaggccaggaaagtgatctcctccctcacggagcggctttactgcgggggccctatgtttaacagcaagggggcccagtgtggttaccgccgttgccgtgccagtggagttctgcctaccagcttcggcaacacgatcacttgttacatcaaggccacagcggccgcaaaggccgcgggcctccggaacccggactttcttgtctgcggagatgatctggtcgtggtggctgagagtgatggcgtcagcgaggatagagcagccctgagagccttcacggaggctatgaccaggtactctgctccacccggagatgctccacagcccacctacgaccttgagctcattacatcttgctcctccaacgtctccgtggcattcgacggcaaggggaagaggtattattacctcacccgtgatgccactacccccctagcccgcgcggcttgggaaacagctcgtcacactccagttaactcctggttgggtaacatcatcatgtacgcgcccaccatctgggtgcgtatggtaatgatgacacattttttctccatactccaatcccaggagatacttgatcgaccccttgactttgaaatgtacggggacacttactctgtcactccgctggatttaccagcaatcattgaaagactccatggtctaagcgcgttcacgctccacagttactctccggtagagctcaatagggttgcggggacactcaggaagcttgggtgcccccccctacgagcttggagacatcgggcacgagcagtgcgcgccaagcttatcgcccagggagggaaggccaagatatgcggcctttatctctttaattgggcggtacgcaccaagaccaacctcactccactgccagccgctggccagctggatttgtccagctggtttacggttggtgtcggcgggaacgacatttatcacagcgtgtcgcgtgcccgaacccgccatttgctgctttacctactcctactaacagtaggggtaggcatcttcctccttccagctcgatgagctggtaggataacactccattcttttttttttttttttttttttttttttttttttttttttttttttttttttttttcttttccttttcccttcttttctgacctttaatcttccttcttaggtggctccatcttagccctagtcacggctagctgtgaaaggtccgtgagccgcatgactgcagagagtgctgatactggcctctctgcagatcatgt

SEQ ID NO: 12 - DNA, 9646 bp, Hepatitis C virus
  source 1..9646: mol_type = genomic DNA
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaagtgccggccgcaccacggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccattgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttcctcctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccttctacgggatgtggcctctcctcctgctcctgctggcgttgcctcagcgggcatacgcactggacacggaggtggccgcgtcgtgtggcggcgttgttcttgtcgggttaatggcgctgactctgtcgccatattacaagcgctacatcagctggtgcatgtggtggcttcagtattttctgaccagagtagaagcgcaactgcacgtgtgggttccccccctcaacgtccggggggggcgcgatgccgtcatcttactcatgtgtgttgtacacccgactctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgcacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaggcggtggactttatccctgtggagaacctagagacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccctttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctacagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttctatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggattgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccaagcagaggttatcacccctgctgtccagaccaactggcagaaactcgaggtcttctgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctaccgcctttgtgggcgctggcttagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacataggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggccagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacccgcagaaattctgcggaagtctcggagattcgcccgggccctgcccgtttgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaggccatttcctgtttttttttttttttttttttttttttttttttttttttttttttttttttctttttttttttttttttccttttttttttttttttttttctttccttcttttttcctttcttttccttccttctttaatggtggctccatcttagccctagtcacggctagctgtgaaaggtccgtgagccgcatgactgcagagagtgctgatactggcctctctgcagatcatgt

SEQ ID NO: 13 - DNA, 20 bp, synthetic construct
  misc_feature 1..20: note = 3' cDNA primer
  source 1..20: mol_type = other DNA
taagaggccggagtgtttac

SEQ ID NO: 14 - DNA, 24 bp, synthetic construct
  misc_feature 1..24: note = 3' cDNA primer
  source 1..24: mol_type = other DNA
ctatggagtgtacctagtgtgtgc

SEQ ID NO: 15 - DNA, 19 bp, synthetic construct
  misc_feature 1..19: note = 3' cDNA primer
  source 1..19: mol_type = other DNA
aaaagaatggagtgttatc

SEQ ID NO: 16 - DNA, 20 bp, synthetic construct
  misc_feature 1..20: note = 3' cDNA primer
  source 1..20: mol_type = other DNA
tgcctgatagggtgcttgcg

SEQ ID NO: 17 - DNA, 20 bp, synthetic construct
  misc_feature 1..20: note = 3' cDNA primer
  source 1..20: mol_type = other DNA
aggccggagtgtttacccca

SEQ ID NO: 18 - DNA, 20 bp, synthetic construct
  misc_feature 1..20: note = 3' cDNA primer
  source 1..20: mol_type = other DNA
tggtctccatctgggaaaag

SEQ ID NO: 19 - DNA, 20 bp, synthetic construct
  misc_feature 1..20: note = 3' cDNA primer
  source 1..20: mol_type = other DNA
cttgcgagtgccccgggagg

SEQ ID NO: 20 - DNA, 26 bp, synthetic construct
  misc_feature 1..26: note = 3' cDNA primer
  source 1..26: mol_type = other DNA
tggagtgtacctagtgtgtgccgctc

SEQ ID NO: 21 - DNA, 26 bp, synthetic construct
  misc_feature 1..26: note = 3'
  source 1..26: mol_type = other DNA
gggatgacatcagcgttccgcgtgac

SEQ ID NO: 22 - DNA, 21 bp, synthetic construct
  misc_feature 1..21: note = 3' cDNA primer
  source 1..21: mol_type = other DNA
gatagggtgcttgcgagtgcc

SEQ ID NO: 23 - DNA, 27 bp, synthetic construct
  misc_feature 1..27: note = 3' cDNA primer
  source 1..27: mol_type = other DNA
agaatggagtgttatcctaccagctca

SEQ ID NO: 24 - DNA, 21 bp, synthetic construct
  misc_feature 1..21: note = 3' cDNA primer
  source 1..21: mol_type = other DNA
ctggccacccaacragrtcct

SEQ ID NO: 25 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTGPASAYQVRNSTGLYHVTNDCPNSSIVFEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHISGHRMAWDMMMNWSPTAALLVAQLLRIPQAILDMIAGAHWGVLAGMAYFSMVGNWAKVLVVLLLFAGVDAETYVTGGSAARTTAGLASLFSPGAKQNIQLVNTNGSWHINSTALNCNDSLNTGWIAGLFYHHKFNSSGCSERLASCRPLTDFAQGWGPISHADGSGPDQRPYCWHYPPKPCGIVPAKSVCGPVYCFTPSPVVVGTTDRSGAPTYSWGANDTDVFVLNNTRPPLGNWFGCTWMNSTGFTKVCGAPPCVIGGVGNNTLRCPTDCFRKHPEATYSRCGSGPWITPRCLVDYPYRLWHYPCTINYTVFKVRMYVGGVEHRLEAACNWTRGDRCNLDDRDRSELSPLLLSTTQWQVLPCSFTTLPALSTGLIHLHQNIVDVQYLYGVGSSIASWAIKWEYVVLLFLLLADARVCSCLWMMLLISQVEAALENLVVLNAASLAGTHGLVSFLVFFCFAWYLKGKWVPGAVYALYGMWPLLLLLLALPQRAYALDTEVAASCGGVVLVGLMALTLSPYYKRYISWCLWWLQYFLTRIEAQLHVWIPPLNVRGGRDAVILLMCVVHPALVFDITKLLLAAFGPLWILQASLLKVPYFVRVQGLLRICALARKMAGGHYVQMAIIKLGALTGTYVYNHLTPLRDWAHNGLRDLAVAVEPVVFSRMETKLITWGADTAACGDIINGLPVSARRGREILLGPADGMVSKGWRLLAPITAYAQQTRGLLGCIITSLTGRDKNQVEGEIQIVSTAAQTFLATCINGVCWTVYHGAGTRTIASPKGPVIQMYTNVDKDLVGWPAPQGARSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVPQSFQVGHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSITVPHPNIEEVALSTTGEIPFYGKAIPLEAIKGGRHLIFCHSKKKCDELAAKLVALGINAVAYYRGLDVSVIPTSGDVVVVATDALMTGYTGDFDSVIDCNTCVTQTVDLSLDPTFTIETTTLPQDAVSRTQRRGRTGRGKPGIYRFVALGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLTRLKPTLHGPTPLLYRLGAVQNEVTLTHPITKYIMTCMSADLEVVTSTWVLVGGVLAALASYCLSTGCVVIVGRIVLSGKPAIIPDREVLYREFDEMEECSQHLPYIEQGMMLAEQFKQKALGLLQTASRQAEVVAPAVQTNWQKLEAFWAKHMWNFISGIHYLAGLSTLPGNPAIASLMAFTAAVTSPLTTSQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGTHVSPTHYVPESDAAARVTTILSSLTVTQLLRRLHQWISSECTTPCSGSWLRDIWDWICEVLSDFKTWLKAKLMPQLPGIPFVSCQRGYKGVWRGDGIMHTRCHCGAEITGHVKNGTMRIVGPKTCRNMWSGTFPINAYTTGPCTPLPAPNYTFALWRVSAEEYVEIRQVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSMASSSASQLSAPSLRATCTTNHDSPDAELIEANLLWRQEMGGNITRVESENKVVILDSFDPLVAEEDEREVSVPAEILRKSRKFTPALPIWARPDYNPPLVEPWKKPDYEPPVVHGCPLPPPQSPPVPPPRKKRTVILTESTLPTALAELATKSFGSSSTSGITGDDTTTSPEPASSSCPPDSDAESYSSMPPLEGEPGDPDLSDGSWSTVSSEADKEDVVCCSMSYTWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVNHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTENDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARNVRARLLSRGGRAAICGKYLFNWAVRTKLKLTPIAAAGRLDLSGWFTAGYSGGGIFHSVSHARPRWFWSCLLLLAAGVGIYLLPNR

SEQ ID NO: 26 - protein, 3033 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNASQCWIPVSPNVAVQRPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIISGAHWGVMFGLAYFSMQGAWAKVVVILLLAAGVDARTHTVGGSAAQTTGRLTSLFDMGPRQKIQLVNTNGSWHINRTALNCNDSLHTGFIASLFYTHSFNSSGCPERMSACRSIEAFRVGWGALQYEDNVTNPEDMRPYCWHYPPRQCGVVSAKTVCGPVYCFTPSPVVVGTTDRLGAPTYTWGENETDVFLLNSTRPPLGSWFGCTWMNSSGYTKTCGAPPCRTRADFNASTDLLCPTDCFRKHPDTTYLKCGSGPWLTPRCLIDYPYRLWHYPCTVNYTIFKIRMYVGGVEHRLTAACNFTRGDRCNLEDRDRSQLSPLLHSTTEWAILPCSYSDLPALSTGLLHLHQNIVDVQFMYGLSPALTKYIVRWEWVILLFLLLADARVCACLWMLILLGQAEAALEKLVILHAASAASCNGFLYFVIFSVAAWYIKGRVVPLATYSLTGLWSFSLLLLALPQQAYAYDASVHGQIGAALLVMITLFTLTPGYKTLLSRFLWWLCYLLTLGEAMVQEWAPPMQVRGGRDGIIWAVAIFYPGVVFDITKWLLAVLGPAYLLKGALTRVPYFVRAHALLRMCTMARHLAGGRYVQMALLALGRWTGTYIYDHLTPMSDWAASGLRDLAVAVEPIIFSPMEKKVIVWGAETAACGDILHGLPVSARLGREVLLGPADGYTSKGWSLLAPITAYAQQTRGLLGTIVVSMTGRDKTEQAGEIQVLSTVTQSFLGTSISGVLWTVYHGAGNKTLAGSRGLVTQMYSSAEGDLVGWPSPPGTKSLEPCTCGAVDLYLVTRNADVIPARRRGDKRGALLSPRPLSTLKGSSGGPVLCPRGHAVGVFRAAVCSRGVAKSIDFIPVETLDIVTRSPTFSDNSTPPAVPQTYQVGYLHAPTGSGKSTKVPVAYAAQGYKVLVLNPSVAATLGFGAYLSKAHGINPNIRTGVRTVTTGAPITYSTYGKFLADGGCAGGAYDIIICDECHAVDSTTILGIGTVLDQAETAGVRLTVLATATPPGSVTTPHPNIEEVALGQEGEIPFYGRAIPLSYIKGGRHLIFCHSKKKCDELAAALRGMGLNSVAYYRGLDVSVIPTQGDVVVVATDALMTGYTGDFDSVIDCNVAVTQVVDLSLDPTFTITTQIVPQDAVSRSQRRGRTGRGRLGIYRYVSTGERASGMFDSVVLCECYDAGAAWYELTPSETTVRLRAYFNTPGLPVCQDHLEFWEAVFTGLTHIDAHFLSQTKQSGENFAYLTAYQATVCARAKAPPPSWDVMWKCLTRLKPTLVGPTPLLYRLGSVTNEVTLTHPVTKYIATCMQADLEVMTSTWVLAGGVLAAVASYCLATGCVCIIGRLHINQRAVVAPDKEVLYEAFDEMEECASRAALIEEGQRIAEMLKSKIQGLLQQASKQAQDIQPTVQASWPKVEQFWAKHMWNFISGIQYLAGLSTLPGNPAVASMMAFSAALTSPLSTSTTILLNILGGWLASQIAPPAGATGFVVSGLVGAAVGSIGLGKVLVDILAGYGAGISGALVAFKIMSGEKPSMEDVVNLLPGILSPGALVVGVICAAILRRHVGPGEGAVQWMNRLIAFASRGTHVAPTHYVTESDASQRVTQLLGSLTITSLLRRLHNWITEDCPIPCGGSWLRDVWDWVCTILTDFKNWLTSKLFPKMPGLPFVSCQKGYKGVWAGTGIMTTRCPCGANISGNVRLGSMRITGPKTCMNIWQGTFPINCYTEGQCVPKPAPNFKVAIWRVAASEYAEVTQHGSYHYITGLTTDNLKVPCQLPSPEFFSWVDGVQIHRFAPTPKPFFRDEVSFCVGLNSFVVGSQLPCDPEPDTDVLMSMLTDPSHITAETAARRLARGSPPSEASSSASQLSAPSLRATCTTHGKAYDVDMVDANLFMGGDVTRIESGSKVVVLDSLDPMVEERSDLEPSIPSEYMLPKKRFPPALPAWARPDYNPPLVESWKRPDYQPATVAGCALPPPRKTPTPPPRRRRTVGLSEDSIGDALQQLAIKSFGQPPPSGDSGLSTGAGAADSGSQTPPDELALSETGSISSMPPLEGELGDPDLEPEQVEPQPPPQGGVAAPGSDSGSWSTCSEEDDSVVCCSMSYSWTGALITPCSPEEEKLPINPLSNSLLRYHNKVYCTTTKSASLRAKKVTFDRMQVLDSYYDSVLKDIKLAASKVTARLLTMEEACQLTPPHSARSKYGFGAKEVRSLSGRAVNHIKSVWKDLLEDSETPIPTTIMAKNEVFCVDPTKGGKKAARLIVYPDLGVRVCEKMALYDITQKLPQAVMGASYGFQYSPAQRVEFLLKAWAEKKDPMGFSYDTRCFDSTVTERDIRTEESIYRACSLPEEAHTAIHSLTERLYVGGPMFNSKGQTCGYRRCRASGVLTTSMGNTITCYVKALAACKAAGIIAPTMLVCGDDLVVISESQGTEEDERNLRAFTEAMTRYSAPPGDPPRPEYDLELITSCSSNVSVALGPQGRRRYYLTRDPTTPIARAAWETVRHSPVNSWLGNIIQYAPTIWARMVLMTHFFSILMAQDTLDQNLNFEMYGAVYSVSPLDLPAIIERLHGLDAFSLHTYTPHELTRVASALRKLGAPPLRAWKSRARAVRASLISRGGRAAVCGRYLFNWAVKTKLKLTPLPEARLLDLSSWFTVGAGGGGIYHSVSRARPRLLLFGLLLLFVGVGLFLLPAR

SEQ ID NO: 27 - protein, 3022 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGHPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCIPCVQDGNASTCWTPVTPTVAVRYVGATTASIRSHVDLLVGAATMCSALYVGDMCGAVFLVGQAFTFRPRRHQTVQTCNCSLYPGHLSGHQMAWDMMMNWSPAVGMVVAHILRLPQTLFDIIAGAHWGILAGLAYYSMQGNWAKVAIIMIMFSGVDADTHTIGGVVARGVRGLSSLLSPGPKQKLELINTNGSWHINSTALNCNDSLNTGFIAGLIYYHKFNATGCPQRLSSCKPITSFGQGWGPLAAANITGSSDDRPYCWHYAPRPCNTVSASSVCGPVYCFTPSPVVVGTTDAKGAPTYNWGANESDVFLLESLRPPSGRWFGCVWMNSTGFVKTCGAPPCDIYGGGENPRNKSDSLRCPTDCFRKHPEATYSRCGAGPWLTPRCMVDYPYRLWHYPCTVNFTLFKARMFVGGFEHRFSAACNWTRGERCNIEDRDRSEQQPLLHSTTELAILPCSFTPMPALSTGLIHLHQNIVDVQYLYGVGSGMVGWALKWEFVVLIFLLLADARVCVALWLMLMITQAEAALENLVTLNAVAAAGTHGIGWYLVAFCAAWYVRGKLVPLVTYSLTGLWSLALLVLLLPQRAYAWSGEDSATLGAGVLILFGFFTLSPWYKHWIGRLMWWNQYAICRCESALQVWVPPLLARGSRDGVILLTSLLYPSLIFDITKLLIAVLGPLYLIQAAITTTPYFVRAHVLVRLCMLVRSMMGGKYLQMIILSIGRWFNTYLYDHLAPLQHWAAAGLKDLAVATEPVIFSPMEIKIITWGADTAACGDILCGLPVSARLGREVLLGPADDYREMGWRLLAPITAYTQQTRGLLGTIVTSLTGRDKNVVTGEVQVLSTATQTFLGTTVGGVMWTVYHGAGSRTLAGVKHPALQMYTNVDQDLVGWPAPPGAKSLEPCACGSADLYLVTRDADVIPARRRGDSTASLLSPRPLACLKGSSGGPVMCPSGHVAGIFRAAVCTRGVAKALQFIPVETLSTQARSPSFSDNSTPPAVPQSYQVGYLHAPTGSGKSTKVPAAYVAQGYNVLVLNPSVAATLGFGSFMSRAYGIDPNIRTGNRTVTTGAKLTYSTYGKFLADGGCSGGAYDVIICDECHAQDATSILGIGTVLDQAETAGVRLTVLATATPPGSITVPHSNIEEVALGSEGEIPFYGKAIPIALLKGGRHLIFCHSKKKCDEIASKLRGMGLNAVAYYRGLDVSVIPTAGDVVVCATDALMTGFTGDFDSVIDCNVAVEQYVDFSLDPTFSIETRTAPQDAVSRSQRRGRTGRGRLGTYRYVTPGERPSGMFDSVVLCECYDAGCSWYDLQPAETTVRLRAYLSTPGLPVCQDHLDFWESVFTGLTHIDAHFLSQTKQQGLNFSYLTAYQATVCARAQAPPPSWDETWKCLVRLKPTLHGPTPLLYRLGPIQNEICLTHPITKYIMACMSADLEVTTSTWVLLGGVLAALAACCLSVGCVVIVGHIELGGKPALVPDKEVLYQQYDEMEECSQAAPYIEQAQVIAHQFKEKVLGLLQRATQQQAVIEPIVVTNWQKLEAFWHKHMWNFASGIQYLAGLSTLPGNPAVASLMAFTASVTSPLTTNQTVFFNILGGWVATHLAGPQSSSAFVVSGMAGAAIGGVGLGRVLLDILAGYGAGVSGALVAFKIMGGEFPTAEDMVNLLPAILSPGALVVGVICAAILRRHVGPGEGAVQWMNRLIAFASRGNHVSPTHYVPESDAAAKVTALLSSLTVTSLLRRLHQWINEDYPSPCSGDWLRDIWDWVCTVLSDFKSWLSAKIMPALPGLPFISCQKGYKGVWRGDGVMSTRCPCGATIAGHVKNGSMRLAGPRTCANMWYGTFPINEYTTGPSTPCPSPNYTRALWRVAASSYVEVRRVGDFHYITGATEDELKCPCQVPAAEFFTEVDGVRLHRYAPPCKPLLRDDITFMVGLNSYAIGSQLPCEPEPDVSVLTSMLRDPSHITAETAARRLARGSPPSEASSSASQLSAPSLKATCQTHRPHPDAELVDANLLWRQEMGSNITRVESETKVVILDSFEPLRAEIDDAELSVAAECFKKPPKYPPALPIWARPDYNPPLLDRWKAPDYEPPTVHGCALPPRGAPPVPPPRRKKTIQLDGSNVSAALAALAEKSFPSSKPQEENSSSSGVDTQSSITSKVPPSPGGESDSESCSSMPPLEGEPGDPDLSCDSWSTVSDSEEQRVVCCSMSYSWTGALITPCSAEEEKLPINPLSNSLLRHHNLVYSTSSRSASQRQKKVTFDRLQVLDDHYKTALKEVKERASKVKARMLTIEEACALVPPHSARSKFGYSAKDVRSLSSKAINQIRSVWEDLLEDTTTPIPTTIMAKNEVFCVDPAKGGRKPARLIVYPDLGVRVCEKRALYDVIQKLSIETMGSAYGFQYSPQQRVERLLKMWTSKKTPLGFSYDTRCFDSTVTEQDIRVEEEIYQCCNLEPEARKVISSLTERLYCGGPMFNSKGAQCGYRRCRASGVLPTSFGNTITCYIKATAAAKAAGLRNPDFLVCGDDLVVVAESDGVSEDRAALRAFTEAMTRYSAPPGDAPQPTYDLELITSCSSNVSVAFDDKGKRYYYLTRDATTPLARAAWETARHTPVNSWLGNIIMYAPTIWVRMVMMTHFFSILQSQEILDRPLDFEMYGDTYSVTPLDLPAIIERLHGLSAFTLHSYSPVELNRVAGTLRKLGCPPLRAWRHRARAVRAKLIAQGGKAKICGLYLFNWAVRTKTNLTPLPAAGQLDLSSWFTVGVGGNDIYHSVSRARTRHLLLYLLLLTVGVGIFLLPAR

SEQ ID NO: 28 - DNA, 9599 bp, Hepatitis C virus
  source 1..9599: mol_type = genomic DNA
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcgcgtcggcccgagggcaggacctgggctcagcccgggtacccttggcccctctatggcaatgagggttgcgggtgggcgggatggctcctgtctccccgtggctctcggcctagctggggccccacggacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttttggaagacggcgtgaactatgcaacagggaaccttcctggttgctctttctctattttccttctggccctgctctcttgtctgactggacccgcttcagcctaccaagtgcgcaactccacggggctttaccatgtcaccaatgattgccccaactcgagcattgtgttcgaggcggctgatgccatcctgcacactccggggtgtgtcccttgcgtacgcgagggtaacgcctcgaggtgttgggtggcggtaacccccacggtggccaccagggatggcaaactccccacaacgcagcttcgacgtcacatcgatctgcttgtcgggagcgccaccctctgctcggccctttacgtgggggacctgtgcgggtctgtctttcttgttggtcaactgttcaccttctctcccaggcgccactggacgacgcaagattgcaattgttctatctaccccggccatatttcaggtcaccgtatggcatgggatatgatgatgaactggtcccctacggcggcgttgttggtagctcagctgctccggatcccacaagccatcctggacatgatcgctggtgctcactggggagtcctggcgggcatggcgtatttctccatggtggggaactgggcgaaggttctggtagtgctgctgctatttgccggcgtcgacgcggaaacctacgtcaccgggggaagtgccgcccgtactacggctggacttgctagtcttttctcaccaggcgccaagcagaacatccagctggtcaacaccaacggcagttggcacatcaatagcacggccttgaactgcaatgacagcctcaacaccggctggatagcaggacttttctatcaccacaaattcaactcttcgggctgttccgagaggttagccagctgccgaccccttaccgattttgcccagggctggggccctatcagtcacgccgacggaagtggccccgaccaacgcccctactgctggcactaccctccaaaaccttgtggtattgtgcccgcaaagagcgtgtgtggcccggtatattgtttcactcccagtcccgtggtggtgggaacgaccgacaggtcgggcgcgcctacctacagctggggtgcaaatgacacggacgtcttcgtccttaacaacaccaggccaccgctgggcaattggttcggttgcacctggatgaactcaactggattcaccaaagtgtgcggagcgcccccttgcgtcatcggaggggtgggcaacaacaccttgcgctgccccactgattgtttccgcaagcatccggaagccacatactctcggtgcggctccggtccctggattacgcccaggtgcctggtcgactacccgtataggctttggcattatccttgtaccatcaactacaccgtgtttaaagtcaggatgtacgtgggaggggtcgagcacaggctggaagctgcctgcaactggacgcggggcgaccgttgtaatctggatgacagggacaggtccgagctcagcccgctgctgctgtccactacgcagtggcaggtcctcccgtgttccttcacgaccctgccagccttgtccaccggcctcatccacctccaccaaaacatcgtggacgtgcaatacttgtacggggtgggatcaagcatcgcgtcctgggccatcaagtgggaatacgtcgttctcttgttccttctgcttgcagacgcgcgcgtctgctcctgcttgtggatgatgttactcatatcccaagtggaggcggctttggagaacctcgtagtactcaatgcagcatccctggccgggacacacggtcttgtatccttcctcgtgttcttctgctttgcatggtatctgaagggtaagtgggtgcccggagcggtctacgccctctacgggatgtggcctctcctcttgctcctgttagcgttgccccagcgggcatacgcactggacacggaggtggccgcgtcgtgtggcggcgttgttcttgtcgggttaatggcgctgactctgtcaccatattacaagcgctatatcagctggtgcttgtggtggcttcagtatttcctgaccagaatagaagcgcaactgcacgtgtggattccccctctcaacgtccggggggggcgcgatgccgtcatcttactcatgtgtgttgtgcacccggctctggtatttgacatcaccaagctactgctggctgccttcgggcccctttggattcttcaagccagtttgcttaaggtaccctacttcgtgcgcgttcaaggccttctccggatctgcgcgctagcgcggaagatggctggaggccattacgtgcaaatggccatcatcaagttaggggcgcttactggcacttatgtttacaaccatctcaccccccttcgggactgggcgcacaacggcctgcgagatctggccgtggctgtggagccagtcgtcttctcccgaatggagaccaagcttatcacctggggggcagacaccgccgcgtgcggtgacatcatcaacggcttgcccgtctccgcccggaggggccgggagatactgctcgggccagccgatggaatggtctccaaggggtggagattgctggcgcccatcacggcgtacgcccagcaaacgaggggcctcctagggtgtataatcaccagtctgaccggccgggacaaaaaccaagtggagggtgagatccagattgtgtcaactgctgcccaaaccttcctggcaacgtgcatcaacggggtttgctggaccgtctaccacggggccggaacgaggaccatcgcatcacccaagggtccggttatccaaatgtataccaatgtggacaaagaccttgtgggctggcccgctcctcaaggtgcccgctcactgacaccctgtacctgcggctcctcggacctttacctggtcacgaggcacgccgatgtcattcccgtgcgccggcggggtgatagcaggggcagcctgctttcgccccggcccatttcctacttgaaaggctcctcggggggtccgctgctgtgccccgcgggacacgccgtaggcttattcagggccgcggtgtgcacccgtggagtggctaaggcggtggacttcatccctgtggagaacctagagacaaccatgaggtccccggtgttcacggacaactcctctccaccagcagtgccccagagcttccaggtgggccacctgcatgctcccaccggcagcggcaaaagcaccaaggtcccggctgcatacgcagctcagggctataaggtgctagtgctcaacccctctgtcgctgcaacactgggctttggtgcttacatgtccaaggcccatggggtcgatcctaatatcaggaccggggtgagaacaattaccactggcagccccatcacgtactccacctacggcaagttccttgccgacggcgggtgttcagggggtgcttatgacataataatttgtgacgagtgccactccacggatgccacatccatcttgggcatcggcaccgttcttgaccaagcagagaccgcgggggcgagactggttgtgctcgccaccgctacccctccgggctccatcaccgtgccccatcctaacatcgaggaggttgctctgtccactaccggagagatccctttttacggcaaggctatccccctcgaggcgatcaagggggggagacatctcatcttctgtcactcaaagaagaagtgcgacgagctcgccgcaaagctggtcgcattgggcatcaatgccgtggcctactaccgcggtcttgacgtgtctgtcatcccaaccagcggcgatgttgtcgtcgtggcgaccgatgctcttatgactggctataccggcgactttgactcggtgatagactgcaacacgtgtgtcacccagacagtcgacctcagccttgaccctaccttcaccattgagacgaccacgctcccccaggacgctgtctcccgcacacaacgccggggcaggactggcagggggaagccaggcatctacagattcgtggcactgggggagcgcccctccggcatgttcgactcgtccgttctctgtgagtgctatgacgcgggctgtgcttggtatgagctcacgcccgccgagactacagttagactacgagcgtacatgaacaccccggggctccctgtgtgccaggaccatcttgaattttgggagggcgtctttacaggcctcacccatatagatgcccatttcctatctcagacaaagcagagcggggaaaactttccttacctggtagcataccaagcaaccgtgtgcgctagggctcaagcccctcccccatcgtgggaccagatgtggaagtgtttgactcgcctcaagcccaccctccatgggccaacacccttgctatacagactgggcgctgttcagaatgaagtcaccctgacgcacccaatcaccaaatacatcatgacatgcatgtcggccgacctggaggtcgtcacgagcacctgggtgctcgtcggcggcgtcctggccgctttggcctcgtattgcctgtcaacaggctgcgtggtcatagtgggcaggattgtcctgtctgggaagccggcaattatacctgacagggaagttctctaccgggagttcgatgagatggaagagtgctctcagcacttaccgtacatcgagcaagggatgatgctcgccgagcagttcaagcagaaggccctcggcctcctgcagaccgcgtcccgtcaggcagaggttgtcgcccctgctgtccagaccaactggcaaaaactcgaggccttctgggcgaagcatatgtggaacttcatcagtgggatacactacttggcgggcttgtcaacgttgcctggtaaccccgccattgcttcattgatggcttttacagctgctgtcaccagcccactaaccactagccaaaccctcctcttcaacatactgggggggtgggtggctgcccagcttgccgcccccggtgccgccaccgcctttgtgggcgctggcttagccggcgccgcaatcggcagtgttggactggggaaggtcctcgtggacattctagcagggtatggcgcgggcgtggcgggagctcttgtagcattcaagatcatgagcggtgaggtcccctccacggaggacctagtcaacctgctgcccgccatcctctcgcctggagcccttgtagtcggtgtggtctgcgcagcaatactgcgccggcacgtcggcccgggcgagggggcagtgcaatggatgaaccggctaatcgccttcgcctcccgggggacccatgtttcccccacgcactatgtgccggagagcgatgcagctgcccgcgtcactaccatactcagcagcctcactgtaacccagctcctgaggcgactgcaccagtggataagctcggagtgtaccactccatgctccggttcctggctgagggacatctgggactggatatgcgaggtgctgagcgactttaagacctggctgaaagccaagctcatgccgcaactgcctgggattccctttgtgtcctgccagcgtgggtataagggggtctggcgaggggacggcatcatgcacactcgctgccactgtggagctgagataactggacatgtcaaaaacgggacgatgaggatcgttggtcctaagacttgcaggaacatgtggagtgggactttccccattaacgcctacaccacgggcccctgtactccccttcctgcgccgaactatacgttcgcgctgtggagggtgtctgcagaggaatacgtggagataaggcaggtgggggacttccactacgtgacgggtatgactactgacaaccttaaatgcccgtgccaggtcccatcgcccgaatttttcacagaattggacggggtgcgcctacataggtttgcgcccccttgcaagcccctgctgcgggaggaggtgtcattcagagtgggactccacgagtacccggtggggtcgcaattaccttgcgagcccgaaccggacgtggccgtgttgacgtccatgctcactgatccctcccatataacagcagaggcggccgggagaaggttggcgaggggatcacccccctctatggccagctcctcggctagccaactgtccgctccatctctcagggcaacttgcactaccaaccatgactcccctgatgctgagctcatagaggccaacctcctatggaggcaggagatgggcggcaacatcaccagggttgagtcagagaacaaagtggtgattctggactccttcgatccgcttgtggcagaggaggatgagcgggaggtctccgtgcccgcagagatactgcggaagtctcggaaattcaccccagccctacccatttgggcgcggccggactataaccccccgctggtggagccgtggaaaaagcctgactacgaaccacctgtggtccatggctgcccgcttccacctccacagtcccctcctgtgcctccacctcggaagaagcggacggtgatcctcaccgaatcaaccctacctactgccttggccgagcttgccaccaaaagttttggcagctcctcaacttccggcattacgggcgacgacacgacaacatcccctgagcccgcctcctctagctgccctcccgactccgacgctgagtcctattcttccatgccccctctggagggggagcctggggatccggatctcagcgacgggtcatggtcgacggtcagtagtgaggccgacaaggaggatgtcgtgtgctgctcaatgtcttatacctggacaggcgcactcgtcaccccgtgcgccgcggaagaacaaaaactgcccatcaacgcactaagcaactcgttgctgcgtcatcacaatctggtgtattccaccacctcacgcagtgcttgccaaaggcagaagaaagtcacatttgacagactgcaagtcctggacagccattaccaggacgtgctcaaggaggttaaggcagcggcgtcaaaagtgaaggctaacttgctatccgtagaggaagcttgcagcctgacgcccccacactcagccaaatccaagtttggctatggggcaaaagacgtccgttgccatgccagaaaggccgtaaaccacatcaactccgtgtggaaagaccttctggaagacagtgtaacaccaatagacactaccatcatggctaagaacgaggttttctgcgttcagcctgagaaggggggtcgtaagccagctcgtctcatcgtgttccccgacctgggtgtacgcgtgtgcgagaagatggccttgtacgacgtagtcagcaagctccccctagccgtgatgggaagctcctacggattccaatactcaccaggacagcgggttgaattcctcgtgcaagcgtggaagtccaagaagaccccaatggggttttcgtatgatacccgctgttttgactccacagtcactgagaatgatatccgtacggaggaggcaatctaccaatgttgtgacctggacccccaagcccgcgtggccatcaagtccctcactgagaggctttatgtcgggggccctcttaccaattcaaggggggagaactgcggctatcgcaggtgccgcgcgagcggcgtactgacgaccagctgtggtaacaccctcacctgctacatcaaggcccgagcagcctgtcgagccgcagggctccaggactgcaccatgctcgtgtgtggcgacgacttagtcgttatctgtgagagtgcgggggtccaggaggacgcggcgagcctgagagccttcacggaggctatgaccaggtactccgccccccccggggaccccccacaaccagaatacgacttggagctcataacatcatgctcctccaacgtgtcagtcgcccacgacggcgctggaaaaagggtctactacctcacccgtgaccctacaacccccctcgcgcgggccgcgtgggagacagcaagacacactccagtcaattcctggctaggcaacataattatgtttgcccccacactgtgggcgaggatgatactgatgacccatttctttagcgtcctcatagccagggatcagcttgaacaggccctcgattgcgagatctacggggcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggaatgtccgcgctaggcttctgtccagaggaggcagggctgccatttgtggcaagtacctcttcaactgggcagtaaggacaaagctcaaactcactccaatagcggccgctggccggctagacttatctggctggttcacggctggctacagcgggggaggcatttttcacagcgtgtctcatgcccggccccgctggttctggtcttgcctactcctgctcgctgcaggggtaggcatctacctcctccccaaccgatgaaggttggggtaaacactccggcctcttaggccatttcctgttttttttttttttttttttttttttttttttttttttttttttttctttttccctctttttcttctctttttccttctttaatggtggctccatcttagccctagtcacggctagctgtgaaaggtccgtgagccgcatgactgcagagagtgctgatactggcctctctgcagatcatgt

SEQ ID NO: 29 - DNA, 9678 bp, Hepatitis C virus
  source 1..9678: mol_type = genomic DNA
acccgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacaccaaccgtcgcccacaagacgttaagtttccgggcggcggccagatcgttggcggagtatacttgttgccgcgcaggggccccaggttgggtgtgcgcgcgacaaggaagacttcggagcggtcccagccacgtggaaggcgccagcccatccctaaagatcggcgctccactggcaaatcctggggaaaaccaggatacccctggcccctatacgggaatgagggactcggctgggcaggatggctcctgtccccccgaggttcccgtccctcttggggccccaatgacccccggcataggtcgcgcaacgtgggtaaggtcatcgataccctaacgtgcggctttgccgacctcatggggtacatccctgtcgtgggcgccccgctcggcggcgtcgccagagctctcgcgcatggcgtgagagtcctggaggacggggttaattttgcaacagggaacttacccggttgctccttttctatcttcttgctggccctgctgtcctgcatcaccaccccggtctccgctgccgaagtgaagaacatcagtaccggctacatggtgactaacgactgcaccaatgacagcattacctggcagctccaggctgctgtcctccacgtccccgggtgcgtcccgtgcgagaaagtggggaatgcatctcagtgctggataccggtctcaccgaatgtggccgtgcagcggcccggcgccctcacgcagggcttgcggacgcacatcgacatggttgtgatgtccgccacgctctgctctgccctctacgtgggggacctctgcggtggggtgatgctcgcagcccaaatgttcattgtctcgccgcagcaccactggtttgtccaagactgcaattgctccatctaccctggtaccatcactggacaccgcatggcatgggacatgatgatgaactggtcgcccacggctaccatgatcttggcgtacgcgatgcgtgtccccgaggtcattatagacatcattagcggggctcattggggcgtcatgttcggcttggcctacttctctatgcagggagcgtgggcgaaagtcgttgtcatccttctgttggccgccggggtggacgcgcgcacccatactgttgggggttctgccgcgcagaccaccgggcgcctcaccagcttatttgacatgggccccaggcagaaaatccagctcgttaacaccaatggcagctggcacatcaaccgcaccgccctgaactgcaatgactccttgcacaccggctttatcgcgtctctgttctacacccacagcttcaactcgtcaggatgtcccgaacgcatgtccgcctgccgcagtatcgaggccttccgggtgggatggggcgccttgcaatatgaggataatgtcaccaatccagaggatatgagaccctattgctggcactacccaccaaggcagtgtggcgtggtctccgcgaagactgtgtgtggcccagtgtactgtttcacccccagcccagtggtagtgggcacgaccgacaggcttggagcgcccacttacacgtggggggagaatgagacagatgtcttcctattgaacagcactcgaccaccgctggggtcatggttcggctgcacgtggatgaactcttctggctacaccaagacttgcggcgcaccaccctgccgtactagagctgacttcaacgccagcacggacctgttgtgccccacggactgttttaggaagcatcctgataccacttacctcaaatgcggctctgggccctggctcacgccaaggtgcctgatcgactacccctacaggctctggcattacccctgcacagttaactataccatcttcaaaataaggatgtatgtgggaggggttgagcacaggctcacggctgcatgcaatttcactcgtggggatcgttgcaacttggaggacagagacagaagtcaactgtctcctttgttgcactccaccacggaatgggccattttaccttgctcttactcggacctgcccgccttgtcgactggtcttctccacctccaccaaaacatcgtggacgtacaattcatgtatggcctatcacctgccctcacaaaatacatcgtccgatgggagtgggtaatactcttattcctgctcttagcggacgccagggtttgcgcctgcttatggatgctcatcttgttgggccaggccgaagcagcactagagaagctggtcatcttgcacgctgcgagcgcagctagctgcaatggcttcctatattttgtcatcttttccgtggctgcttggtacatcaagggtcgggtagtccccttagctacctattccctcactggcctgtggtcctttagcctactgctcctagcattgccccaacaggcttatgcttatgacgcatctgtgcatggccagataggagcggctctgctggtaatgatcactctctttactctcacccccgggtataagacccttctcagccggtttttgtggtggttgtgctatcttctgaccctgggggaagctatggtccaggagtgggcaccacctatgcaggtgcgcggtggccgtgatggcatcatatgggccgtcgccatattctacccaggtgtggtgtttgacataaccaagtggctcttggcggtgcttgggcctgcttacctcctaaaaggtgctttgacgcgcgtgccgtacttcgtcagggctcacgctctactgaggatgtgcaccatggcaaggcatctcgcggggggcaggtacgtccagatggcgctactagcccttggcaggtggactggcacttacatctatgaccacctcacccctatgtcggattgggctgctagtggcctgcgggacctggcggtcgccgttgagcctatcatcttcagtccgatggagaagaaagtcattgtctggggagcggagacagctgcttgtggggacattttacacggacttcccgtgtccgcccgacttggtcgggaggtcctccttggcccagctgatggctatacctccaaggggtggagtcttctcgcccccatcactgcttacgcccagcagacacgtggccttttgggcaccatagtggtgagcatgacggggcgcgacaagacagaacaggctggggaaattcaggtcctgtccacagtcactcagtccttcctcggaacatccatctcgggggttttgtggactgtctaccatggagctggcaacaagactctggccggctcacggggtctggtcacgcagatgtactccagtgctgagggggacttagtagggtggcccagcccccctgggactaaatctttggagccgtgcacgtgtggagcggtcgacctgtacctggtcacgcggaacgctgatgtcatcccggctcgaagacgcggggacaaacggggagcgctactctccccgagacctctttccaccttgaaggggtcctcaggaggcccggtgctatgccccaggggccacgctgtcggagtcttccgggcagctgtgtgctctcggggcgtggctaagtccatagatttcatccccgttgagacactcgacatcgtcacgcggtcccccacctttagtgacaacagcacaccacctgctgtgccccagacctatcaggtcgggtacttgcatgccccgactggcagtggaaagagcaccaaagttcctgtcgcatatgctgctcaggggtataaagtgctagtgcttaatccctcagtggctgccaccctggggtttggggcgtacttgtctaaggcacatggcatcaatcccaacattaggactggagtcaggactgtgacgaccggggcgcccatcacgtactccacatatggcaaattcctcgccgatgggggctgtgcgggcggcgcctacgacatcatcatatgtgatgaatgccatgccgtggactctaccaccatccttggcatcggaacagtccttgatcaagcagagacagctggggtcagactaactgtgctggctacagctacgccccctgggtcagtgacaaccccccaccccaacatagaggaggtggcccttgggcaggagggcgagatccccttctatgggagggcgattcccctgtcttacatcaagggaggaagacatctgatcttctgccattcaaagaaaaagtgtgacgagctcgcggcggcccttcggggtatgggcttgaactcagtggcatactacagagggttggacgtctccgtaataccaactcagggagacgtagtggtcgtcgccaccgacgccctcatgacagggtatactggggactttgactccgtgatcgactgcaacgtagcggtcactcaagttgtagacctcagtttagaccccacattcaccataaccacacagattgtccctcaagacgctgtctcacgtagccagcgccggggtcgcacgggtaggggaagactgggcatttataggtatgtttccactggtgagcgagcctcaggaatgtttgacagtgtagtgctctgtgagtgctacgacgcaggggccgcatggtatgagctcacaccatcggagaccaccgtcaggctcagggcgtatttcaacacgcccggtttgcctgtgtgccaagaccatcttgagttttgggaggcagttttcaccggcctcacacacatagatgcccacttcctttcccaaacaaagcaatcgggggaaaatttcgcatacttaacagcctaccaggctacagtgtgcgctagggccaaagccccccccccgtcctgggacgtcatgtggaagtgtttgactcgactcaagcccacactcgtgggccccacacctctcctgtaccgcttgggctctgttaccaacgaggtcaccctcacacatcccgtgacgaaatacatcgccacctgcatgcaagccgaccttgaggtcatgaccagcacatgggtcttggcagggggagtcttggcggccgtcgcctcgtattgcctggcgaccgggtgtgtttgcatcatcggccgcttgcacattaaccagcgagccgtcgttgcgccggacaaggaggtcctctatgaggcttttgatgagatggaggaatgtgcctctagggcggctctcattgaagaggggcagcggatagccgagatgctgaagtccaagatccaaggcttattgcagcaagcttccaaacaagctcaagacatacaacccactgtgcaggcttcatggcccaaggtagaacaattctgggccaaacacatgtggaacttcattagcggcatccaatacctcgcaggactatcaacactgccagggaaccctgcagtagcttccatgatggcgttcagtgccgccctcaccagtccgctgtcaacaagcaccactatccttctcaacattttggggggctggctagcatcccaaattgcaccacccgcgggggccactggcttcgttgtcagtggcctagtgggagctgccgtaggcagtataggcttaggtaaggtgctagtggacatcctggcagggtatggtgcgggcatttcgggggctctcgtcgcattcaagatcatgtctggcgagaagccctccatggaggatgtcgtcaacttgctgcctggaattctgtctccgggtgccttggtagtgggagtcatctgcgcggccattctgcgccgacacgtgggaccgggggaaggcgccgtccaatggatgaatagactcattgcctttgcttccagaggaactcacgtcgcccccacccactacgtgacggagtcggatgcgtcgcagcgtgtgacccaactacttggctcccttaccataaccagcctgctcagaagactccacaactggattactgaggactgccccatcccatgcggcggctcgtggctccgcgatgtgtgggactgggtttgcaccatcctaacagactttaaaaattggctgacctccaaattattcccaaagatgcccggcctcccctttgtctcctgtcaaaaggggtacaagggcgtgtgggccggcactggcatcatgaccacacggtgtccttgcggcgccaatatctctggcaatgtccgcttgggctccatgagaatcacggggcctaagacctgcatgaatatctggcaggggacctttcctatcaattgttacacggagggccagtgcgtgccgaaacccgcgccaaactttaaggtcgccatctggagggtggcggcctcagagtacgcggaggtgacgcagcacgggtcataccactacataacaggactcaccactgataacttgaaagtcccctgccaactaccctctcccgagttcttttcctgggtggacggagtgcagatccataggtttgcccccacaccgaagccgtttttccgggatgaggtctcgttctgcgttgggcttaattcatttgtcgtcgggtcccagcttccttgcgaccctgaacccgacacagacgtattgatgtccatgctaacagatccatctcatatcacggcggagactgcagcgcggcgtttagcgcgggggtcacccccatccgaggcaagctcctcggcgagccagctatcggcaccatcgctgcgagccacctgcaccacccacggcaaagcctatgatgtggacatggtggatgctaacctgttcatggggggcgatgtgactcggatagagtctgggtccaaagtggtcgttctggactctctcgacccaatggtcgaagaaaggagcgaccttgagccttcgataccatcagaatacatgctccccaagaagaggttcccaccagctttaccggcctgggcacggcctgattacaacccaccgcttgtggaatcgtggaaaaggccagattaccaaccggccactgttgcgggctgtgctctccctcctcctaggaaaaccccgacgcctcccccaaggaggcgccggacagtgggcctaagtgaggactccataggagatgcccttcaacagctggccattaagtcctttggccagccccccccaagcggcgattcaggcctttccacgggggcgggcgctgccgattccggcagtcagacgcctcctgatgagttggccctttcggagacaggttccatctcttccatgccccccctcgagggggagcttggagatccagacctggagcctgagcaggtagagccccaaccccccccccaggggggggtggcagctcccggctcggactcggggtcctggtctacttgctccgaggaggacgactccgtcgtgtgctgctccatgtcatactcctggaccggggctctaataactccttgtagtcccgaagaggagaagttaccgattaaccccttgagcaactccctgttgcgatatcacaacaaggtgtactgtaccacaacaaagagcgcctcactaagggctaaaaaggtaacttttgataggatgcaagtgctcgactcctactacgactcagtcttaaaggacattaagctagcggcctccaaggtcaccgcaaggctcctcaccatggaggaggcttgccagttaaccccaccccattctgcaagatctaaatatgggtttggggctaaggaggtccgcagcttgtccgggagggccgttaaccacatcaagtccgtgtggaaggacctcctggaggactcagaaacaccaattcccacaaccattatggccaaaaatgaggtgttctgcgtggaccccaccaaggggggcaagaaagcagctcgccttatcgtttaccctgacctcggcgtcagggtctgcgagaagatggccctttatgacattacacaaaaacttcctcaggcggtgatgggggcttcttatggattccagtattcccccgctcagcgggtagagtttctcttgaaagcatgggcggaaaagaaggaccctatgggtttttcgtatgatacccgatgctttgactcaaccgtcactgagagagacatcaggactgaggagtccatatatcgggcctgctccttgcccgaggaggcccacactgccatacactcgctaactgagagactttacgtgggagggcctatgttcaacagcaagggccaaacctgcgggtacaggcgttgccgcgccagcggggtgctcaccactagcatggggaacaccatcacatgctacgtgaaagccttagcggcttgtaaagctgcagggataatcgcgcccacaatgctggtatgcggcgatgacttggttgtcatctcagaaagccaggggaccgaggaggacgagcggaacctgagagccttcacggaggctatgaccaggtattctgcccctcctggtgacccccccagaccggagtatgatctggagctgataacatcttgctcctcaaatgtgtctgtggcgctgggcccacaaggccgccgcagatactacctgaccagagaccctaccactccaatcgcccgggctgcctgggaaacagttagacactcccctgtcaattcatggctgggaaacatcatccagtacgccccgaccatatgggctcgcatggtcctgatgacacacttcttctccattctcatggctcaagacacgctggaccagaacctcaactttgagatgtacggagcggtgtactccgtgagtcccttggacctcccagctataattgaaaggttacatgggcttgacgctttttctctgcacacatacactccccacgaactgacacgggtggcttcagccctcagaaaacttggggcgccacccctcagagcgtggaagagccgggcacgtgcagtcagggcgtccctcatctcccgtggggggagagcggccgtttgcggtcgatatctcttcaattgggcggtgaagaccaagctcaaactcactccattgccggaagcgcgcctcctggatttatccagctggttcaccgtcggcgccggcgggggcggcatttatcacagcgtgtcgcgtgcccgaccccgcttattgctctttggcctactcctactttttgtaggggtaggccttttcctactccccgctcggtagagcggcacacattagctacactccatagctaactgtccctttttttttttttttttttttttttttttttttttttttttttttttttttctttttttctcttttccttctttcttaccttattttactttctttcctggtggctccatcttagccctagtcacggctagctgtgaaaggtccgtgagccgcatgactgcagagagtgccgtaactggtctctctgcagatcatgt

SEQ ID NO: 30 - DNA, 9629 bp, Hepatitis C virus
  source 1..9629: mol_type = genomic DNA
gcctgcctcttacgaggcgacactccaccatggatcactcccctgtgaggaacttctgtcttcacgcggaaagcgcctagccatggcgttagtacgagtgtcgtgcagcctccaggaccccccctcccgggagagccatagtggtctgcggaaccggtgagtacaccggaatcgctggggtgaccgggtcctttcttggagcaacccgctcaatacccagaaatttgggcgtgcccccgcgagatcactagccgagtagtgttgggtcgcgaaaggccttgtggtactgcctgatagggtgcttgcgagtgccccgggaggtctcgtagaccgtgcaacatgagcacacttcctaaacctcaaagaaaaaccaaaagaaacaccatccgccgcccacaggacgtcaagttcccgggtggcggacagatcgttggtggagtatacgtgttgccgcgcaggggcccacgattgggtgtgcgcgcgacgcgtaaaacttctgaacggtcacagcctcgcggacgacgacagcctatccccaaggcgcgtcggagcgaaggccggtcctgggctcagcccgggcacccctggcccctctatggtaacgagggctgcgggtgggcagggtggctcctgtccccacgcggttcccgcccatcttggggcccgaacgacccccggcgaaggtcccgcaatttgggtaaggtcatcgataccctcacgtgtggattcgccgacctcatggggtacatcccgctcgtcggcgctcccgtagggggcgtcgcaagggcccttgcgcatggcgtgagggctcttgaagacgggataaacttcgcaacagggaatttgcccggttgctccttttctatcttcctccttgctctgttctcttgcttaatccatccagcagccagtttagagtggaggaacacgtctggtctctatgtcctcactaacgactgttccaatagcagcattgtgtatgaggccgatgacgttatcctgcacacacccggctgtataccttgtgttcaggacggcaatgcatctacgtgctggaccccagtgacacctacagtggcagtcaggtacgtcggagcaaccaccgcttcgatacgcagtcacgtggacctattagtgggcgcggccacgatgtgctctgcgctctacgtaggtgatatgtgtggggccgtcttcctcgtgggacaagccttcacgttcagacctcgtcgccatcagacggtccagacctgcaactgctcgctgtacccaggccatctttcaggacaccaaatggcttgggatatgatgatgaattggtcccccgctgtgggtatggtggtggcgcacatcctgcggttgccccagaccttgttcgacataatagccggagctcattggggcatcttggcgggcctggcctattactccatgcagggcaactgggccaaggtcgctatcatcatgattatgttttcgggggtcgatgccgacacacacaccattggtggtgtcgtagctcgtggagtcagaggactttcttctttgctcagtccgggccccaagcagaaactggagctgatcaacaccaacggatcgtggcacatcaatagtactgccctgaactgcaatgattccctaaacaccgggttcatagccgggctgatctattatcataagtttaacgccactggatgtcctcaaaggctcagcagctgcaagcccatcacttccttcgggcaggggtggggccccttggcagctgctaacatcaccggctcttctgatgacagaccgtactgctggcactacgcacctagaccttgtaacacagtctcggcatcaagtgtctgcggccctgtgtactgcttcacgccgtcaccggtggtcgtgggcactactgatgctaagggcgcgccaacctacaactggggtgcgaatgagtcagacgtgttcctgctggaatccctgcggcctcccagtggtcggtggtttgggtgcgtgtggatgaactccacgggatttgtcaagacgtgcggagcccctccttgtgatatctatgggggtggggaaaatccccggaataagtcagactccctccgctgccccaccgactgcttcaggaaacatcctgaggccacatacagccggtgtggcgcggggccctggttgacacctcggtgcatggtagactacccataccggctttggcattacccatgtacagtcaattttacattgttcaaggcgaggatgtttgtaggtgggtttgagcaccggttctccgccgcctgcaactggaccaggggggagcgctgcaatatcgaggatcgtgaccgcagcgaacaacaaccgctgctgcattcaacaactgagcttgctatactgccttgctctttcacgcccatgcctgcgttgtcaacaggtctgatacacctccaccaaaacatcgtggatgtccaatacctttatggcgttggatctggcatggtgggatgggcgttgaagtgggagttcgtagtcctcattttcctcctcctagcagacgcacgcgtgtgcgttgccctttggctgatgctgatgataacgcaagcggaagcagccctggagaaccttgtcacgctgaatgctgtcgctgctgctgggacacatggtatcggctggtacctggtagccttttgcgcagcgtggtacgtgcggggtaaacttgtcccgctagtgacctacagcctgacgggactttggtccctggcactactcgttctcctgctcccccaacgggcgtatgcttggtcgggtgaggacagcgctactctcggtgctggggtcttgatcctcttcggcttctttaccttatcaccttggtacaagcattggatcggccgcctcatgtggtggaaccagtacgccatatgcaggtgcgagtccgcccttcaagtgtgggtcccccccctacttgcacgcgggagcagggacggtgtcatcctgctaacaagcctgctttatccatctttaatttttgacatcactaagctgctgatagcggtattgggcccattatacttgatacaggctgccatcactactaccccctattttgtgcgtgcgcatgtactggttcgcctttgcatgctcgtgcgctccatgatggggggaaaatacctccagatgatcatactgagcataggcaggtggttcaacacctacttatacgaccacctagcgccattgcaacactgggccgcagccggtctcaaagacctagcagtggccactgagcctgtaatattcagtcccatggaaatcaagatcatcacctggggcgcggacacagcagcttgtggagatattctttgcgggctgcccgtctctgcgcgattaggccgtgaggtattattgggacctgctgatgactatcgggagatgggttggcgcctgttggccccgatcacagcatacacccagcaaactaggggccttcttgggactattgtgaccagcttgactggcagggataagaacgtggtgaccggtgaagtgcaggtgctttctacggctacccagaccttcctaggtacaacagtagggggggttatgtggactgtttaccatggtgcaggctcgagaacactcgcgggcgtcaaacatcctgcgctccaaatgtacacaaatgtggatcaggaccttgttgggtggccagcccctccgggggctaagtctctcgaaccgtgcgcctgcggatctgcagacttatacttggttacccgcgatgctgatgtcatccctgctcggcgcaggggagactccacagcgagcttgctcagtcctaggcctcttgcctgccttaagggttcttctggaggtcctgttatgtgcccgtcggggcatgttgcggggatatttagggctgctgtgtgcaccagaggcgtagcaaaagccctacagttcataccagtggaaacccttagcacacaggctaggtctccatctttctctgacaattcaactcctcctgccgtcccacagagctatcaagtcgggtaccttcatgccccgaccggtagtggtaagagcacaaaggtcccggccgcttatgtagcgcaaggatataatgttcttgtgttgaatccatcggtggcggccacactaggcttcggctctttcatgtcgcgtgcctatgggatcgaccctaatatccgcactgggaaccgtaccgttacaactggtgctaaactgacctattccacctatggtaagtttcttgcggacgggggttgctccgggggagcatatgatgtgatcatctgtgacgagtgtcatgcccaagacgctactagcatattgggtataggcacggtcttagatcaggctgagacggctggggtgaggctgacggttctagcgacagcaactcccccaggcagcatcactgtgccacattctaacatcgaagaggtggccctaggctctgaaggtgagatccctttctacggtaaggctataccgatagccctgcttaagggggggagacaccttatcttttgccattccaagaaaaaatgtgatgagatagcatccaaactcagaggcatggggctcaacgctgtagcgtactataggggtctcgatgtgtccgtcataccaacagcaggagacgtcgtagtttgcgctactgacgccctcatgactgggttcaccggagacttcgattctgtcatagattgcaacgtagctgttgaacagtacgttgacttcagcctggaccccactttttccattgagactcgcactgctccccaagacgcggtttcccgcagccagcgtcgtggccgtacgggccgaggtagactcggcacgtaccgatatgtcacccccggtgaaagaccgtctggaatgtttgactcggttgttctctgtgagtgctatgacgcgggctgctcgtggtacgatctgcagcccgctgagaccacagttagactgagagcttacttgtccacgccggggttacctgtctgccaagaccatttggacttctgggagagcgtcttcactggactaacacacatagatgcccattttctgtcacagactaagcagcagggacttaacttctcgtacctgactgcctaccaagccactgtgtgcgcccgcgcgcaggctcctcccccaagttgggacgagacgtggaagtgtctcgtgcggcttaagccaacactacatggacctacgccccttctgtatcggttggggcctatccaaaatgaaatctgtttgacacatcccatcacaaaatacatcatggcatgcatgtcagctgatctggaagtgaccaccagcacctgggtgctgcttggaggggtccttgcggccctagcggcttgctgcttgtcagtcggctgcgttgtgattgtgggccatatcgagctggggggcaagccagcacttgttccagacaaagaggtgttgtatcaacaatacgatgagatggaggagtgctcgcaagccgccccatacatcgaacaagctcaggtaatagcccaccagttcaaggaaaaagtccttggattgctgcagcgagccacccaacaacaagctgtcattgagcccatagtagttaccaactggcaaaagcttgaggccttctggcataagcatatgtggaactttgcgagtgggatccagtacctggcaggcctctccaccttgcctggcaaccctgctgtggcgtctcttatggcgttcactgcttcagtcaccagccccctgacgaccaaccaaactgtgttttttaacatactcggggggtgggttgctacccatttggcagggccccagagctcttctgcattcgtggtaagtggcatggccggcgctgccatagggggtgtaggcctgggcagggtcttgcttgacatcctggcaggatacggagctggtgtctcaggtgccttggtggcttttaagatcatgggaggagaattccccactgctgaagacatggtcaacctgttgcccgccatactatctccgggtgctctcgtcgtcggtgtgatatgcgctgccatactacgtcgccacgtaggacctggagagggggcggtacagtggatgaacagactcattgcgttcgcatcccggggcaaccacgtctcaccaacgcactatgtccccgagagcgatgctgcagcaaaggttaccgcattgctcagttctctaactgtcacgagtctgctccggcggttgcaccagtggatcaatgaggactacccaagtccttgcagcggcgattggttgcgtgacatctgggactgggtttgtacagtgttgagtgactttaaatcatggctctctgctaagattatgccagcgctccctggactgcctttcatttcctgtcaaaagggatataagggcgtgtggcggggggacggtgtgatgtcaacgcgctgtccttgcggagcaacaatagccggccatgtgaagaatgggtccatgcggcttgcggggccgcgtacatgtgctaacatgtggtacggtactttccccatcaatgagtacaccaccggacccagcacaccttgcccatcacccaactacactcgcgcactatggcgcgtggctgctagcagctacgtcgaggtgcgccgggtgggggacttccattatattacgggggccacagaagatgagctcaagtgtccgtgccaagtgccggctgctgagttctttactgaagtggatggggtaagactccaccgttacgcccctccatgtaagcccctgttgagagatgatatcactttcatggtggggttgaattcctacgctatagggtcccaactcccctgtgagcccgaaccagacgtttctgtgctgacctcgatgttgagagacccctctcatatcaccgccgagacggcagcgcgccgccttgcgcgcggatcccctccatcagaggcgagctcatccgccagccaattgtcggctccgtcgttgaaggccacttgccagacgcataggcctcatccagacgctgagctagtggatgccaacttgttatggcggcaagagatgggtagcaacatcacacgggtagagtctgagacgaaggttgtgattcttgattcattcgaacctctgagagccgaaattgatgacgctgagctctcggtggctgcagagtgtttcaagaaacctcccaagtatcctccagcccttcccatctgggctaggccagactataaccctccactgctcgaccgctggaaagcaccagattatgaaccaccaactgtccatggatgtgccttaccaccgcgaggcgctccaccggtgcctccccctcggaggaagaaaacaattcagctggatggttccaatgtgtccgcggcgctagctgcgctggcggaaaaatcatttccatcctcgaaaccacaggaagagaatagctcatcctctggggtcgacacacagtccagcattacttccaaggtgcccccttctccgggaggggagtccgactcagagtcgtgttcgtctatgcctcctctcgagggagagccgggcgatccagacttgagttgtgactcctggtccaccgttagtgacagcgaggagcagagagtggtctgctgctctatgtcatactcctggaccggcgccctaataacaccatgtagtgctgaggaggagaaactgcccatcaacccactcagcaattccttgttgagacatcataacctagtctattcaacgtcgtctcgaagcgcttctcagcggcagaagaaggttaccttcgacaggctgcaggtgctcgacgaccattacaagactgcattaaaggaggtaaaggagcgggcgtccaaggtaaaggcccgcatgcttaccatcgaggaagcgtgcgcgctcgtccctcctcactctgcccggtcgaagttcgggtatagtgcgaaggacgtccgctccttgtccagcaaagccattaaccagatccgctccgtctgggaggacttgctggaagacaccacaaccccaattccaactaccataatggcgaagaacgaggtattctgtgtggaccccgctaaggggggccgcaagcccgctcgcctcattgtgtaccctgacctgggggtgcgtgtctgtgagaaacgtgccctatatgacgtgatacagaagttgtcaattgagacgatggggtctgcctacggattccaatactcgcctcaacagcgggtcgaacgtctgctgaagatgtggacctcaaagaagacccccttggggttctcgtatgacacccgctgctttgactcgactgtcactgaacaggacatcagggtggaagaggagatataccaatgctgtaatcttgaaccggaggccaggaaagtgatctcctccctcacggagcggctttactgcgggggccctatgtttaacagcaagggggcccagtgtggttaccgccgttgccgtgccagtggagttctgcctaccagcttcggcaacacgatcacttgttacatcaaggccacagcggccgcaaaggccgcgggcctccggaacccggactttcttgtctgcggagatgatctggtcgtggtggctgagagtgatggcgtcagcgaggatagagcagccctgagagccttcacggaggctatgaccaggtactctgctccacccggagatgctccacagcccacctacgaccttgagctcattacatcttgctcctccaacgtctccgtggcattcgacgacaaggggaagaggtattattacctcacccgtgatgccactacccccctagcccgcgcggcttgggaaacagctcgtcacactccagttaactcctggttgggtaacatcatcatgtacgcgcccaccatctgggtgcgtatggtaatgatgacacattttttctccatactccaatcccaggagatacttgatcgaccccttgactttgaaatgtacggggacacttactctgtcactccgctggatttaccagcaatcattgaaagactccatggtctaagcgcgttcacgctccacagttactctccggtagagctcaatagggttgcggggacactcaggaagcttgggtgcccccccctacgagcttggagacatcgggcacgagcagtgcgcgccaagcttatcgcccagggagggaaggccaagatatgcggcctttatctctttaattgggcggtacgcaccaagaccaacctcactccactgccagccgctggccagctggatttgtccagctggtttacggttggtgtcggcgggaacgacatttatcacagcgtgtcgcgtgcccgaacccgccatttgctgctttacctactcctactaacagtaggggtaggcatcttcctccttccagctcgatgagctggtaggataacactccattcttttttttttttttttttttttttttttttttttttttttttttttttttttttttcttttccttttcccttcttttctgacctttaatcttccttcttaggtggctccatcttagccctagtcacggctagctgtgaaaggtccgtgagccgcatgactgcagagagtgctgatactggcctctctgcagatcatgt

(19) United States
(12) Patent Application Publication
(10) Pub. No.: US 2026/0263580 A1
(43) Pub. Date: Sep. 10, 2026

(54) HIGH-YIELD GENOTYPE 1a, 2a AND 3a HCV

(71) Applicant: Hvidovre Hospital, Hvidovre (DK)

(72) Inventors: Garazi Pena Alzua, Hvidovre (DK); Anne Finne Pihl, Hvidovre (DK); Anna Offersgaard, Hvidovre (DK); Ulrik Fahnøe, Hvidovre (DK); Jens Bukh, Hvidovre (DK); Judith Margarete Gottwein, Hvidovre (DK)

(21) Appl. No.: 18/876,529

(22) PCT Filed: Jun. 30, 2023
(86) PCT No.: PCT/EP2023/068038

§ 371 Date: Dec. 18, 2024

(30) Foreign Application Priority Data
Jun. 30, 2022 (EP) 22182368.5

(51) Int. Cl. A61K 39/12; A61K 39/00; A61P 31/14; C12N 7/00; C12Q 1/70

(52) U.S. Cl. CPC A61K 39/12; A61P 31/14; C12N 7/00; C12Q 1/707; A61K 2039/53; A61K 2039/575; A61K 2039/70; C12N 2770/24234; C12N 2770/24251

(57) ABSTRACT

The present invention relates to nucleic acid sequences that encode high-yield hepatitis C viruses (HCV) of genotype 1a, 2a or 3a that are useful in the fundamental research of HCV as well as in the search of antivirals and vaccines against HCV. In particular, the present invention relates to nucleic acid sequences that comprise HCV, which are capable of expressing the virus when transfected into cells and are capable of replication or infectivity in cultured cells as well as being functional as a vaccine.

### TECHNICAL FIELD OF THE INVENTION

The present invention provides high-yield hepatitis C genotype 1a, 2a and 3a viruses (HCV), and vectors, cells and animals comprising the same. The present invention provides methods of producing the high-yield HCV genotype 1a, 2a and 3a, and their use in identifying anti-HCV therapeutics including use in vaccines.

### BACKGROUND OF THE INVENTION

Hepatitis C virus (HCV) is a highly prevalent, blood borne enveloped positive-sense single strand RNA virus of the Flaviviridae family. In contrast to the nonstructural proteins (p7, NS2, NS3, NS4A, NS4B, NS5A and NS5B), the structural proteins, capsid protein Core and envelope glycoproteins E1 and E2, are contained in the viral particle. The E1/E2 heterodimer is the main target for neutralizing antibodies (nAb). Among eight major genotypes differing in ~30% of their sequence, genotypes 1, 2 and 3 cause >80% of infections worldwide. Genotypes 4, 5 and 6 show a more restricted geographic localization in the Middle East/Africa, South Africa and Southeast Asia, respectively, while genotypes 7 and 8 were reported in few individuals. Subtypes (a, b, c, etc.) differ in ~20% of their sequence.

Each year, ~1.5 million new infections occur. Of these, ~80% proceed to chronic infection resulting in a total of ~58 million chronically infected individuals with increased risk for liver cirrhosis and hepatocellular carcinoma, causing ~290,000 deaths annually. So far antiviral treatment did not have a major impact on this epidemic, mainly due to lack of symptoms prior to severe liver damage, lack of screening programs and high cost of treatment in many countries. A prophylactic vaccine will be required to reach the WHO aim to eliminate hepatitis as a major public health threat.

During natural infection, T and B cells appear to contribute to protective immunity. A T-cell vaccine using a viral vector approach did not protect against chronic infection in chimpanzees and humans. In contrast, a B-cell vaccine based on E1/E2 glycoprotein heterodimers showed protective effects in chimpanzees and induced nAb in non-human primates, chimpanzees and humans, even though nAb were induced in <50% of human vaccine recipients with limited capacity to neutralize different HCV genotypes. Induction of nAb is considered to correlate with efficacy of other viral vaccines. Moreover, during natural HCV infections induction of broadly nAb mediated protection. Protective nAb targeted conserved conformational neutralizing epitopes in E2 and E1/E2 localizing to antigenic regions 3 and 4 (AR3 and AR4), also targets of well-defined human monoclonal antibodies (mAb) (Law et al., 2008; Giang et al., 2012). For efficacy against different HCV genotypes a future vaccine should target such epitopes, which, however, appear to be hidden by closed envelope protein conformational states (E1/E2 states) (Prentoe et al., 2019; Augestad et al., 2020). Another approach might be a multivalent vaccine based on different viral variants.

For HCV, in mice whole virus vaccines showed a higher capacity to induce nAb than protein-based vaccines, presumably due to higher density and more native conformation of the envelope proteins. Indeed, many licensed viral vaccines are based on whole viruses or virus like particles. However, application of this technology in HCV vaccine development has been hampered by relatively low viral yields in cell culture systems for production of HCV. In 2005, the first systems were developed based on a single genotype 2a isolate (JFH1) (Lindenbach et al., 2005), followed by JFH1-based systems expressing genotype specific proteins (Gottwein et al., 2009) and eventually full-length systems not depending on JFH1 genetic elements (Li et al., 2012a; Li et al., 2012b; Ramirez et al., 2016). These systems typically yield 103-105 infectious viruses per ml, considered suboptimal for vaccine development. Nevertheless, proof-of-concept for immunogenicity of a JFH1-based genotype 2a recombinant was obtained in mice and non-human primates (Akazawa et al., 2013; Yokokawa et al., 2018). However, efficient nAb were only induced with adjuvants not licensed for human use.

Accordingly, improved cell culture systems capable of producing viruses for effective vaccine development would be advantageous, and in particular a more efficient and/or reliable system in order to obtain immunogenicity in a vaccine context would be advantageous.

### SUMMARY OF THE INVENTION

Thus, an object of the present invention relates to development of high-yield cell culture system for production of HCV, such as production of genotype 1a, 2a and 3a HCV.

A further object of the present invention relates to the development of vaccines capable of introducing immunogenicity, such as vaccines for human use.

Thus, one aspect of the invention relates to an isolated nucleic acid molecule which encodes human hepatitis C virus or a fragment hereof,
wherein said hepatitis C virus is derived from genotype 1a and said nucleic acid molecule encodes an amino acid sequence comprising one of the following groups of adaptive mutations:
   a) S2482G, N2651H, 12902L and H2986R according to SEQ ID NO: 25; or
   b) T2357A, S2375G, C2419R, M2834L and H2986R according to SEQ ID NO: 25;
wherein said hepatitis C virus is derived from genotype 2a and said nucleic acid molecule encodes an amino acid sequence comprising the following adaptive mutations: K78E, V140L, T329S, H434N, A575T, A760T, V773A, V828A, A1146V, I1330V, L1500P, V1835I, T2000A, L2363P, A2367P, E2783Q and L3013S according to SEQ ID NO: 26; or
wherein said hepatitis C virus is derived from genotype 3a and said nucleic acid molecule encodes an amino acid sequence comprising the following adaptive mutations: G395R, A856T, G875R, N1589S, I1753S, V2428A and D2807G according to SEQ ID NO: 27.

Another aspect of the present invention relates to a composition comprising a nucleic acid molecule as described herein suspended in a suitable amount of a pharmaceutical acceptable diluent or excipient.

Yet another aspect of the present invention is to provide a cell comprising the nucleic acid molecule as described herein or the composition as described herein.

Still a further aspect of the present invention relates to a hepatitis C virus particle obtainable by a method comprising culturing a cell as described herein to allow the cell to produce the virus. A related aspect is a hepatitis C virus particle obtainable by a method comprising culturing a cell according to claim 7 under conditions that permit hepatitis C virus replication and virus particle formation.

An even further aspect of the present invention relates to a mixture of hepatitis C virus particles as described herein comprising hepatitis C virus particles of at least two genotypes selected from the group consisting of genotype 1a, genotype 2a and genotype 3a.

Another aspect of the present invention relates to a hepatitis C virus vaccine comprising a nucleic acid molecule or the encoded amino acid sequence or a fragment hereof as described herein; a hepatitis C virus particle as described herein or a part thereof; a mixture of hepatitis C virus particles as described herein; or a mixture of one or more hereof.

Yet another aspect of the present invention relates to a hepatitis C virus particle as described herein, a mixture of hepatitis C virus particles as described herein or a hepatitis C virus vaccine as described herein for use in vaccination of a subject against hepatitis C virus.

An even further aspect of the present invention relates to an antibody against a nucleic acid molecule or the encoded amino acid sequence or a fragment hereof as described herein; a hepatitis C virus particle as described herein or a part thereof; or a mixture of hepatitis C virus particles as described herein; or a mixture of one or more hereof.

A further aspect of the present invention relates to an antibody as described herein, a hepatitis C virus particle as described herein, a mixture of hepatitis C virus particles as described herein or a hepatitis C virus vaccine as described herein for use in treating, alleviating or preventing a hepatitis C virus infection.

Still a further aspect of the present invention relates to a use of a nucleic acid molecule or the encoded amino acid sequence or a fragment hereof as described herein; a hepatitis C virus particle as described herein or a part thereof; a mixture of hepatitis C virus particles as described herein; or a mixture of one or more hereof; or a hepatitis C virus vaccine as described herein for raising neutralizing antibodies and/or raising cross-neutralizing antibodies. A related aspect is the use of a nucleic acid molecule or the encoded amino acid sequence or a fragment hereof as described herein; a hepatitis C virus particle as described herein or a part thereof; a mixture of hepatitis C virus particles as described herein; or a mixture of one or more hereof; or a hepatitis C virus vaccine as described herein for raising neutralizing antibodies and/or raising cross-neutralizing antibodies, with the proviso that the antibodies are raised in a non-human species.

Yet another aspect of the present invention relates to a method for screening an anti-hepatitis C virus substance, comprising
a) culturing at least one selected from the group consisting of a cell comprising the nucleic acid molecule as described herein or a cell obtained by a method comprising producing a cell, which replicates human hepatitis C virus and optionally produces a virus particle, comprising introducing a nucleic acid molecule as described herein into a cell;
together with a hepatitis C virus permissive cell; and
b) detecting the replicating RNA or the virus particles in the resulting culture.

### BRIEF DESCRIPTION OF THE FIGURES

FIG. 1 shows in vitro serial passage resulting in high-yield polyclonal genotype 1a, 2a and 3a HCV. Huh7.5 cells were inoculated with cell culture supernatant containing the specified viruses derived from the previous passage culture at the peak of infection, as determined by immunostaining. Every 2-3 days passage cultures were split and % HCV antigen positive cells and HCV infectivity titers were determined by immunostaining and infectivity titration, respectively. Peak supernatant HCV infectivity titers determined for the specified passages are means of three replicates with standard deviation (SD). For TNcc, the later passage line was inoculated with passage 10 virus from an initial passage line. Black solid bars, passage subjected to NGS. Hatched bar, passage used for production of genotype 2a seed stock. White bar, passage subjected to NGS and used for production of genotype 1a seed stock. The genotype 3a seed stock was based on the later developed DBNcc-HI recombinant.

FIG. 2 shows nucleotide changes in serially passaged genotype 1a, 2a and 3a HCV. For TNcc-PP-38.1, J6cc-PP-35 and DBNcc-PP-16 coding and silent changes with allele frequency >10% determined by NGS of the complete ORF are shown. Coding changes with frequency of ≥80% are specified above bars. Genome positions relate to TNcc (Li et al., 2012a), J6cc (Li et al., 2012b) and DBN3acc (Ramirez et al., 2016) (Genbank accession numbers JX993348, JQ745650, and KX280714, respectively). For encoded amino acid changes, see Tables 4, 5, and 6.

FIG. 3 shows that HCV genotype 1a recombinants engineered based on an initial passage line had increased viral fitness in cell culture. (A) Original TNcc and newly engineered TNcc-HI-18A and TNcc-HI-18B HCV recombinants were transfected into Huh7.5 cells using the same amount of HCV RNA in vitro transcripts. (B) First passage kinetic experiments were inoculated at MOI 0.003 with TNcc and HI-viruses using supernatants derived from the transfection experiment when peak infectivity titers were observed. Cultures were split every 2-3 days. On these days HCV infectivity titers given in log 10 FFU/ml were determined with an infectivity titration assay as means of three replicates with standard deviations (SD). *, cell culture was closed due to HCV induced cell death.

FIG. 4 shows engineered high-yield genotype 1a, 2a and 3a HCV recombinants having increased viral fitness in cell culture. (A) Specified recombinants were transfected into Huh7.5 cells using the same amount of HCV RNA in vitro transcripts for recombinants directly compared in each graph. (B) In first passage kinetic experiments, cells were infected at multiplicity of infection of 0.002 with original and HI-viruses using supernatants derived from the transfection experiment when peak infectivity titers were observed; polyclonal passage viruses were included for comparison. (A, B) Every 2-3 days passage cultures were split and % HCV antigen positive cells and HCV infectivity titers were determined by immunostaining and infectivity titration, respectively. HCV infectivity titers are means of three replicates with SD.

FIG. 5 shows engineered high-yield genotype 1a, 2a and 3a HCV recombinants showing increased sensitivity to neutralization by human derived nAb. Recombinants with in vivo derived genotype (isolate) 1a (TN) (Scheel et al., 2011), 2a (J6) (Lindenbach et al., 2005) and 3a (DBN) (Scheel et al., 2011) Core-NS2 sequences, as well as TNcc-HI, J6cc-HI, DBNcc-HI, the genotype 1a HCV seed stock and DBN3acc (Ramirez et al., 2016) with envelope protein substitutions acquired during in vitro passage were subjected to neutralization with human mAb AR3A and AR4A (Law et al., 2008; Giang et al., 2012), and polyclonal antibody C211 (Prentoe et al., 2012). Datapoints are means of three replicates with SD; curves were fitted and EC50 were calculated with the formula y=100/(1+10^{(log 10EC50-X)×hillslope}) using GraphPad Prism. Fold increase in neutralization sensitivity was calculated as [(EC50 of 1a (TN), 2a (J6) or 3a (DBN) virus with in vivo derived envelope protein sequence)/(EC50 of respective virus with in vitro derived envelope protein substitutions)] and is shown in Tables 7, 8, and 9. Virus stock envelope protein sequences were confirmed by Sanger sequencing.

FIG. 6 shows production of genotype 1a, 2a, and 3a vaccine viruses. Huh7.5 cells were infected with genotype 1a, 2a and 3a HCV seed stocks at MOI 0.003 and transferred to 10 layer-cell factories. For each genotype four cell-factories were processed as indicated by differently colored bars. Per cell factory, five harvests of 800 ml supernatant were collected at the indicated days post cell seed into the cell-factory. HCV infectivity titers in harvests were determined in log 10 FFU/ml and are means of three replicates with SD. Thus, per virus 16L HCV containing supernatant were produced and pooled for further processing.

FIG. 7 shows absence of HCV antigen positive cells in cell cultures inoculated with UV inactivated HCV. Three Huh7.5 cell cultures plated the previous day at 80,000 cells per well in a 24-well dish, were inoculated with 100 μl of UV irradiated HCV of the specified genotype. HCV infection control cultures were inoculated with a genotype 5a HCV (Mathiesen et al., 2015). Negative control cultures were not inoculated. Cultures inoculated with irradiated HCV and negative control cultures were followed for 3 weeks by splitting every 2-3 days and determination of the percentage of HCV antigen positive cells by immunostaining for HCV Core and NS5A. HCV infection control cultures were followed until peak of infection. Percentages of HCV antigen positive cells are means of immunostainings from three replicate cell cultures with SD. For the negative control datapoints were nudged by 0.5 units in the x direction.

FIG. 8 shows immunization with inactivated genotype 1a, 2a or 3a HCV elicited antibodies neutralizing cell culture infectious HCV of the same genotype. Groups of three mice were immunized with inactivated genotype 1a, 2a and 3a HCV or OVA formulated with adjuvant AddaVax. Purified serum IgG from individual mice was used to neutralize recombinants containing in vivo derived genotype (isolate) 1a (TN) (Scheel et al., 2011), 2a (J6) (Lindenbach et al., 2005) and 3a (S52) (Gottwein et al., 2009) specific Core-NS2. Datapoints are means of three replicates with SD; curves were fitted and EC50 were calculated with the formula y=100/(1+10^{(log 10EC50-X)×hillslope}) using GraphPad Prism. Each concentration-response curve specified by unique symbols represents data from one animal. Each vaccine was used to immunize 3 animals. Virus stock envelope protein sequences were confirmed by Sanger sequencing.

FIG. 9 shows immunization with inactivated genotype 1a, 2a or 3a HCV elicited broadly nAb. Purified IgG from mice of each group, immunized with either genotype 1a, 2a or 3a HCV, was pooled using the same amount of IgG from each animal. IgG pools were used to neutralize recombinants containing in vivo derived genotype (isolate) 1a (TN), 1b (J4), 2a (J6), 2b (J8), 3a (S52), 4a (ED43), and 5a (SA13) Core-NS2 sequences; 6a (HK6a) contained two vital cell culture adaptive substitution in E1 and E2 (Lindenbach et al., 2005; Gottwein et al., 2009; Scheel et al., 2011). Datapoints are means of three replicates with SD; curves were fitted and EC50 were calculated with the formula y=100/(1+10^{(log 10EC50-X)×hillslope}) using GraphPad Prism. Virus stock envelope protein sequences were confirmed by Sanger sequencing.

FIG. 10 shows that vaccine induced nAb responses favorably compare to those in patients with chronic HCV infection. Purified IgG from patients chronically infected with HCV of genotype 1a, 2a/2b, or 3a at the specified concentrations was used to neutralize recombinants containing in vivo derived genotype (isolate) 1a (TN), 2a (J6), 3a (S52), and 5a (SA13) Core-NS2 sequences (Lindenbach et al., 2005; Gottwein et al., 2009; Scheel et al., 2011). Genotype 2a versus 2b patients are indicated by grey versus black open circles. Datapoints obtained in neutralization assays with the same concentrations of purified mouse IgG shown in FIG. 6 are re-plotted for comparison. All datapoints are means of three replicates. Virus stock envelope protein sequences were confirmed by Sanger sequencing.

FIG. 11 shows that immunization with inactivated genotype 1a, 2a or 3a HCV elicited antibodies binding to HCV envelope proteins. Binding capacity of pooled (A) purified serum IgG or (B and C) immune-sera from mice immunized with inactivated genotype 1a, 2a or 3a HCV or OVA to E1/E2 complexes of the specified HI-recombinants was evaluated by ELISA. Values are OD reads at 450 nm following subtraction of mean OD of eight negative controls. Datapoints are means of two replicates with SD. (A) Positive controls: Instead of serum IgG, well-characterized primary antibodies were used: AP33 (Clayton et al., 2002) for binding to TNcc-HI and DBNcc-HI E1/E2 and H77.39 (Sabo et al., 2011) for binding to J6cc-HI E1/E2. Negative controls: No IgG or immune-sera were used and TNcc-HI, J6cc-HI and DBNcc-HI E1/E2 were incubated with secondary antibody only; for negative controls, OD reads were ~0.05. (A and B) In the OVA graphs, datapoints reflecting binding to J6cc-HI E1/E2 and DBNcc-HI E1/E2 were nudged by 0.04 and 0.08 units in the y direction, respectively. (C) Immune-sera endpoint titers were determined as the highest serum dilution yielding an OD>2-fold mean OD of negative controls.

FIG. 12 shows that immunization with inactivated genotype 1a, 2a or 3a HCV elicited IgG binding to HCV envelope proteins approaching a binding plateau. Binding capacity of pooled purified serum IgG from mice immunized with inactivated genotype 1a, 2a or 3a HCV or OVA to E1/E2 complexes of the homologous HI-recombinants was evaluated by ELISA. Values are OD reads at 450 nm following subtraction of mean OD of 8 negative controls. Datapoints are means of two replicates with SD. Positive controls: Instead of serum IgG, well-characterized primary antibodies were used: AP33 (Clayton et al., 2002) for binding to TNcc-HI and DBNcc-HI E1/E2 and H77.39 (Sabo et al., 2011) for binding to J6cc-HI E1/E2. Negative controls: No IgG was used and TNcc-HI, J6cc-HI and DBNcc-HI E1/E2 were incubated with secondary antibody only; for negative controls, OD reads were ~0.05. Data are shown both with logarithmic and linear x axis. Compared to FIG. 11, higher IgG concentrations were used to evaluate if a binding plateau could be approached.

FIG. 13 shows immune-sera binding to HCV envelope proteins and determination of endpoint titers. Binding capacity of immune-sera from mice immunized with inactivated genotype 1a, 2a or 3a HCV or OVA to E1/E2 complexes of the specified HI-recombinants was evaluated by ELISA. (A) Vaccine Genotype 1a HCV; (B) Vaccine Genotype 2a HCV; (C) Vaccine Genotype 3a HCV. Values are OD reads at 450 nm following subtraction of mean OD of 8 negative controls. Datapoints are means of two replicates with SD. Negative controls: No immune-sera were used and TNcc-HI, J6cc-HI and DBNcc-HI E1/E2 were incubated with secondary antibody only; for negative controls, OD reads were ~0.05. Broken-lines: 2-fold mean OD of negative controls, used as cut-off for determination of endpoint titers. Endpoint titers are shown in FIG. 11C.

The present invention will now be described in more detail in the following.

### DETAILED DESCRIPTION OF THE INVENTION

The present invention advantageously provides high-yield hepatitis C virus (HCV), of genotypes 1a, 2a and 3a, nucleotide sequences capable of replication, expression of functional HCV proteins, and infection in cells for development of antiviral therapeutics, diagnostics, and vaccines.

Nucleic Acid Molecules (cDNA Clones and RNA Transcripts)

The present invention is directed towards an isolated nucleic acid molecule.

Thus, in one aspect the present invention relates to an isolated nucleic acid molecule which encodes human hepatitis C virus or a fragment hereof,
wherein said hepatitis C virus is derived from genotype 1a and said nucleic acid molecule encodes an amino acid sequence comprising one of the following groups of adaptive mutations:
   a) S2482G, N2651H, 12902L and H2986R according to SEQ ID NO: 25; or
   b) T2357A, S2375G, C2419R, M2834L and H2986R according to SEQ ID NO: 25;
wherein said hepatitis C virus is derived from genotype 2a and said nucleic acid molecule encodes an amino acid sequence comprising the following adaptive mutations: K78E, V140L, T329S, H434N, A575T, A760T, V773A, V828A, A1146V, I1330V, L1500P, V1835I, T2000A, L2363P, A2367P, E2783Q and L3013S according to SEQ ID NO: 26; or
wherein said hepatitis C virus is derived from genotype 3a and said nucleic acid molecule encodes an amino acid sequence comprising the following adaptive mutations: G395R, A856T, G875R, N1589S, 11753S, V2428A and D2807G according to SEQ ID NO: 27.

In one embodiment, the hepatitis C virus is derived from genotype 1a, strain TNcc and optionally, said nucleic acid molecule encodes an amino acid sequence with a sequence identity of at least 95% to that of SEQ ID NO: 25 or a fragment hereof; the hepatitis C virus is derived from genotype 2a, strain J6cc and optionally, said nucleic acid molecule encodes an amino acid sequence with a sequence identity of at least 95% to that of SEQ ID NO: 26 or a fragment hereof; and/or the hepatitis C virus is derived from genotype 3a, strain DBN3acc and optionally, said nucleic acid molecule encodes an amino acid sequence with a sequence identity of at least 95% to that of SEQ ID NO: 27 or a fragment hereof.

In another embodiment, the hepatitis C virus is derived from genotype 1a, strain TNcc and optionally, said nucleic acid molecule encodes an amino acid sequence with a sequence identity of at least 95% to that of SEQ ID NO: 4 or a fragment hereof; the hepatitis C virus is derived from genotype 2a, strain J6cc and optionally, said nucleic acid molecule encodes an amino acid sequence with a sequence identity of at least 95% to that of SEQ ID NO: 5 or a fragment hereof; and/or the hepatitis C virus is derived from genotype 3a, strain DBN3acc and optionally, said nucleic acid molecule encodes an amino acid sequence with a sequence identity of at least 95% to that of SEQ ID NO: 6 or a fragment hereof.

In a further embodiment, the nucleic acid molecule as described encodes an amino acid sequence with a sequence identity of at least 96%, such as 97%, e.g. 98%, such as 99%, e.g. 100% sequence identity to that of SEQ ID NOs: 4, 5, 6, 25, 26 or 27; or a fragment hereof.

In a further aspect, the present invention relates to an isolated nucleic acid molecule which encodes human hepatitis C virus of genotype 1a, strain TNcc; genotype 2a, strain J6cc; or genotype 3a, strain DBN3acc, wherein said nucleic acid molecule encodes an amino acid sequence with a sequence identity of at least 95% to that of SEQ ID NOs: 4, 5, 6, 25, 26 or 27 or a fragment hereof, and wherein the amino acid sequence
further comprises one of the following groups of adaptive mutations:
   a) S2482G, N2651H, 12902L and H2986R according to SEQ ID NO: 25;
   b) T2357A, S2375G, C2419R, M2834L and H2986R according to SEQ ID NO: 25;
   c) K78E, V140L, T329S, H434N, A575T, A760T, V773A, V828A, A1146V, I1330V, L1500P, V1835I, T2000A, L2363P, A2367P, E2783Q and L3013S according to SEQ ID NO: 26; or
   d) G395R, A856T, G875R, N1589S, 11753S, V2428A and D2807G according to SEQ ID NO: 27.

In one embodiment, said amino acid sequence further comprises the following adaptive mutations: T11N, G32S, V365A, F403L, N410K, V719I, V787A, V859A, R1008Q, A1341V, C1457S, G1909A, L2130I and V2417A according to SEQ ID NO: 25. In a further embodiment, said nucleic acid molecule encodes an amino acid sequence derived from genotype 1a and said adaptive mutations of group a). In an even further embodiment, said amino acid sequence further comprises the following adaptive mutations: L179P and S1930Y according to SEQ ID NO: 25.

In a further embodiment, said amino acid sequence further comprises one or more of the following adaptive mutations: L139I or A1309V according to SEQ ID NO: 25 or N415D, E858A and C2441R according to SEQ ID NO: 26.

In an even further embodiment, said amino acid sequence further comprises L139I and/or A1309V according to SEQ ID NO: 25, if derived from genotype 1a.

In a still further embodiment, said amino acid sequence further comprises N415D, E858A and/or C2441R according to SEQ ID NO: 26, if derived from genotype 2a.

Thus, in a further aspect the present invention relates to an isolated nucleic acid molecule which encodes human hepatitis C virus of genotype 1a, strain TNcc, wherein said nucleic acid molecule encodes an amino acid sequence with a sequence identity of at least 95% to that of SEQ ID NO: 4, or a fragment hereof, wherein said nucleic acid molecule encodes an amino acid sequence comprising the following adaptive mutations S2482G, N2651H, 12902L and H2986R according to SEQ ID NO: 25.

Thus, in a further aspect the present invention relates to an isolated nucleic acid molecule which encodes human hepatitis C virus of genotype 1a, strain TNcc, wherein said nucleic acid molecule encodes an amino acid sequence with a sequence identity of at least 95% to that of SEQ ID NO: 25, or a fragment hereof, wherein said nucleic acid molecule encodes an amino acid sequence further comprising the following adaptive mutations S2482G, N2651H, 12902L and H2986R according to SEQ ID NO: 25.

Thus, in a further aspect the present invention relates to an isolated nucleic acid molecule which encodes human hepatitis C virus of genotype 1a, strain TNcc, wherein said nucleic acid molecule encodes an amino acid sequence with a sequence identity of at least 95% to that of SEQ ID NO: 4, or a fragment hereof, wherein said nucleic acid molecule encodes an amino acid sequence comprising the following adaptive mutations T2357A, S2375G, C2419R, M2834L and H2986R according to SEQ ID NO: 25.

Thus, in a further aspect the present invention relates to an isolated nucleic acid molecule which encodes human hepatitis C virus of genotype 1a, strain TNcc, wherein said nucleic acid molecule encodes an amino acid sequence with a sequence identity of at least 95% to that of SEQ ID NO: 25, or a fragment hereof, wherein said nucleic acid molecule encodes an amino acid sequence further comprising the following adaptive mutations T2357A, S2375G, C2419R, M2834L and H2986R according to SEQ ID NO: 25.

Thus, in a further aspect the present invention relates to an isolated nucleic acid molecule which encodes human hepatitis C virus of genotype 1a, strain TNcc, wherein said nucleic acid molecule encodes an amino acid sequence with a sequence identity of at least 95% to that of SEQ ID NO: 4, or a fragment hereof, wherein said nucleic acid molecule encodes an amino acid sequence comprising the following adaptive mutations T11N, G32S, V365A, F403L, N410K, V719I, V787A, V859A, R1008Q, A1341V, C1457S, G1909A, L2130I, V2417A, S2482G, N2651H, 12902L and H2986R according to SEQ ID NO: 25.

Thus, in a further aspect the present invention relates to an isolated nucleic acid molecule which encodes human hepatitis C virus of genotype 1a, strain TNcc, wherein said nucleic acid molecule encodes an amino acid sequence with a sequence identity of at least 95% to that of SEQ ID NO: 25, or a fragment hereof, wherein said nucleic acid molecule encodes an amino acid sequence further comprising the following adaptive mutations T11N, G32S, V365A, F403L, N410K, V719I, V787A, V859A, R1008Q, A1341V, C1457S, G1909A, L2130I, V2417A, S2482G, N2651H, I2902L and H2986R according to SEQ ID NO: 25.

Thus, in a further aspect the present invention relates to an isolated nucleic acid molecule which encodes human hepatitis C virus of genotype 2a, strain J6cc, wherein said nucleic acid molecule encodes an amino acid sequence with a sequence identity of at least 95% to that of SEQ ID NO: 5, or a fragment hereof, wherein said nucleic acid molecule encodes an amino acid sequence comprising the following adaptive mutations K78E, V140L, T329S, H434N, A575T, A760T, V773A, V828A, A1146V, I1330V, L1500P, V1835I, T2000A, L2363P, A2367P, E2783Q and L3013S according to SEQ ID NO: 26.

Thus, in a further aspect the present invention relates to an isolated nucleic acid molecule which encodes human hepatitis C virus of genotype 2a, strain J6cc, wherein said nucleic acid molecule encodes an amino acid sequence with a sequence identity of at least 95% to that of SEQ ID NO: 26, or a fragment hereof, wherein said nucleic acid molecule encodes an amino acid sequence further comprising the following adaptive mutations K78E, V140L, T329S, H434N, A575T, A760T, V773A, V828A, A1146V, I1330V, L1500P, V1835I, T2000A, L2363P, A2367P, E2783Q and L3013S according to SEQ ID NO: 26.

Thus, in a further aspect the present invention relates to an isolated nucleic acid molecule which encodes human hepatitis C virus of genotype 3a, strain DBN3acc, wherein said nucleic acid molecule encodes an amino acid sequence with a sequence identity of at least 95% to that of SEQ ID NO: 6, or a fragment hereof, wherein said nucleic acid molecule encodes an amino acid sequence comprising the following adaptive mutations G395R, A856T, G875R, N1589S, 11753S, V2428A and D2807G according to SEQ ID NO: 27.

Thus, in a further aspect the present invention relates to an isolated nucleic acid molecule which encodes human hepatitis C virus of genotype 3a, strain DBN3acc, wherein said nucleic acid molecule encodes an amino acid sequence with a sequence identity of at least 95% to that of SEQ ID NO: 27, or a fragment hereof, wherein said nucleic acid molecule encodes an amino acid sequence further comprising the following adaptive mutations G395R, A856T, G875R, N1589S, I1753S, V2428A and D2807G according to SEQ ID NO: 27.

In a further aspect the present invention relates to an isolated nucleic acid molecule which encodes human hepatitis C virus or a fragment hereof,
wherein said hepatitis C virus is derived from genotype 1a and said nucleic acid molecule comprises one of the following groups of adaptive mutations:
   a) A7785G, A8292C, A9045C and A9298G according to SEQ ID NO: 28;
   b) A7410G, A7464G, T7596C, A8841T and A9298G according to SEQ ID NO: 28; or
   c) C373A, G435A, T1435C, T1548C, C1571G, G2496A, T2701C, T2917C, G3364A, C4363T, G4711C, G6067C, C6729A, T7591C, A7785G, A8292C, A9045C and A9298G according to SEQ ID NO: 28;
wherein said hepatitis C virus is derived from genotype 2a and said nucleic acid molecule comprises the following adaptive mutations: A572G, G758C, A1325T, C1640A, G2063A, G2618A, T2658C, T2823C, C3777T, A4328G, T4839C, G5843A, A6338G, T7428C, G7439C, G8687C and T9378C according to SEQ ID NO: 29; or
wherein said hepatitis C virus is derived from genotype 3a and said nucleic acid molecule comprises the following adaptive mutations: G1522A, G2905A, G2962A, A5105G, T5597G, T7622C and A8759G according to SEQ ID NO: 30.

In one embodiment, the hepatitis C virus is derived from genotype 1a, strain TNcc and optionally, said nucleic acid molecule has a nucleic acid sequence with a sequence identity of at least 95% to that of SEQ ID NO: 9 or 28, or a fragment hereof; the hepatitis C virus is derived from genotype 2a, strain J6cc and optionally, said nucleic acid molecule wherein said molecule has a nucleic acid sequence with a sequence identity of at least 95% to that of SEQ ID NO: 10 or 29, or a fragment hereof; and/or the hepatitis C virus is derived from genotype 3a, strain DBN3acc and optionally, said nucleic acid molecule wherein said molecule has a nucleic acid sequence with a sequence identity of at least 95% to that of SEQ ID NO: 11 or 30, or a fragment hereof.

In a further embodiment, the nucleic acid molecule as described has a nucleic acid sequence with a sequence identity of at least 96%, such as 97%, e.g. 98%, such as 99%, e.g. 100% sequence identity to that of SEQ ID NO: 9-11 or 28-30 or a fragment hereof.

Throughout the description the substitutions as described herein are to be interpreted as for example for A1146V that alanine (A) at amino acid position 1146 is changed to valine (V), L2130I that leucine (L) at the amino acid position 2130 is changed to isoleucine (I) and so forth.

Thus, A1146V according to SEQ ID NO: 26 is to be interpreted that alanine (A) at amino acid position 1146 in SEQ ID NO: 26 would be changed to valine (V).

Throughout the description the meaning of the adaptive mutations as described herein is to be interpreted as for example for A5105G that adenine (A) at nucleic acid position 5105 is changed to guanine (G) and so forth.

Thus, A5105G according to SEQ ID NO: 30 is to be interpreted that adenine (A) at nucleotide position which would align to nucleotide position 5105 in SEQ ID NO: 30 would be changed to guanine (G).

The terms “isolate” and “strain” are used herein interchangeably.

Thus, one aspect of the present invention relates to an isolated nucleic acid molecule which encodes a high-infective human hepatitis C virus wherein the hepatitis C virus is derived from genotype 1a, 2a or 3a.

The present inventors have identified a wide variety of isolates that generated different virus viability.

These isolates are described in the examples of the present application and are disclosed in the sequence listing as SEQ ID NO: 2-6 (amino acid sequences) and SEQ ID NO: 7-11 (nucleic acid sequences).

In an embodiment of the present invention, these sequences are isolated nucleic acid sequences and amino acid sequence, respectively.

In one embodiment, the molecule as described herein encodes for strain TNcc-HI-18A (SEQ ID NO: 2), strain TNcc-HI-18B (SEQ ID NO: 3), strain TNcc-HI (SEQ ID NO: 4), strain J6cc-HI (SEQ ID NO: 5) or strain DBNcc-HI (SEQ ID NO: 6).

In a further embodiment, said nucleic acid molecule encodes an amino acid sequence according to SEQ ID NO: 4 and wherein said amino acid sequence further comprises the following adaptive mutations: L179P and S1930Y according to SEQ ID NO: 25.

Another aspect of the present invention relates to an isolated nucleic acid molecule encoding for strain TNcc-HI-18A (SEQ ID NO: 2). Another aspect relates to an isolated amino acid molecule TNcc-HI-18A (SEQ ID NO: 2). Another embodiment relates to a nucleic acid molecule encoding an amino acid sequence with a sequence identity of at least 80% to that of SEQ ID NO: 2.

In another embodiment, the nucleic acid molecule encoding an amino acid sequence with a sequence sharing at least 85% identity with that set forth in SEQ ID NO: 2, such as 90% identity, 91% identity, 92% identity, 93% identity, 94% identity, 95% identity, 96% identity, 97% identity, 98% identity, or 99% identity.

Another aspect of the present invention relates to an isolated nucleic acid molecule encoding for strain TNcc-HI-18B (SEQ ID NO: 3). Another aspect relates to an isolated amino acid molecule TNcc-HI-18B (SEQ ID NO: 3). Another embodiment relates to a nucleic acid molecule encoding an amino acid sequence with a sequence identity of at least 80% to that of SEQ ID NO: 3.

In another embodiment, the nucleic acid molecule encoding an amino acid sequence with a sequence sharing at least 85% identity with that set forth in SEQ ID NO: 3, such as 90% identity, 91% identity, 92% identity, 93% identity, 94% identity, 95% identity, 96% identity, 97% identity, 98% identity, or 99% identity.

Another aspect of the present invention relates to an isolated nucleic acid molecule encoding for strain TNcc-HI (SEQ ID NO: 4). Another aspect relates to an isolated amino acid molecule TNcc-HI (SEQ ID NO: 4). Another embodiment relates to a nucleic acid molecule encoding an amino acid sequence with a sequence identity of at least 80% to that of SEQ ID NO: 4.

In another embodiment, the nucleic acid molecule encoding an amino acid sequence with a sequence sharing at least 85% identity with that set forth in SEQ ID NO: 4, such as 90% identity, 91% identity, 92% identity, 93% identity, 94% identity, 95% identity, 96% identity, 97% identity, 98% identity, or 99% identity.

Another aspect of the present invention relates to an isolated nucleic acid molecule encoding for strain J6cc-HI (SEQ ID NO: 5). Another aspect relates to an isolated amino acid molecule J6cc-HI (SEQ ID NO: 5). Another embodiment relates to a nucleic acid molecule encoding an amino acid sequence with a sequence identity of at least 80% to that of SEQ ID NO: 5.

In another embodiment, the nucleic acid molecule encoding an amino acid sequence with a sequence sharing at least 85% identity with that set forth in SEQ ID NO: 5, such as 90% identity, 91% identity, 92% identity, 93% identity, 94% identity, 95% identity, 96% identity, 97% identity, 98% identity, or 99% identity.

Another aspect of the present invention relates to an isolated nucleic acid molecule encoding for strain DBNcc-HI (SEQ ID NO: 6). Another aspect relates to an isolated amino acid molecule DBNcc-HI (SEQ ID NO: 6). Another embodiment relates to a nucleic acid molecule encoding an amino acid sequence with a sequence identity of at least 80% to that of SEQ ID NO: 6.

In another embodiment, the nucleic acid molecule encoding an amino acid sequence with a sequence sharing at least 85% identity with that set forth in SEQ ID NO: 6, such as 90% identity, 91% identity, 92% identity, 93% identity, 94% identity, 95% identity, 96% identity, 97% identity, 98% identity, or 99% identity.

In a further embodiment, the molecule is strain TNcc-HI-18A (SEQ ID NO: 7), strain TNcc-HI-18B (SEQ ID NO: 8), strain TNcc-HI (SEQ ID NO: 9), strain J6cc-HI (SEQ ID NO: 10) or strain DBNcc-HI (SEQ ID NO: 11).

Another aspect of the present invention relates to an isolated nucleic acid molecule being strain TNcc-HI-18A (SEQ ID NO: 7). Another aspect of the present invention relates to an isolated nucleic acid molecule being strain TNcc-HI-18B (SEQ ID NO: 8). Another aspect of the present invention relates to an isolated nucleic acid molecule being strain TNcc-HI (SEQ ID NO: 9). Another aspect of the present invention relates to an isolated nucleic acid molecule being strain J6cc-HI (SEQ ID NO: 10). Another aspect of the present invention relates to an isolated nucleic acid molecule being strain DBNcc-HI (SEQ ID NO: 11).

As commonly defined “identity” is here defined as sequence identity between genes or proteins at the nucleotide or amino acid level, respectively. Thus, in the present context “sequence identity” is a measure of identity between proteins at the amino acid level and a measure of identity between nucleic acid at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. To determine the percent identity of two amino acid sequences or of two nucleic acids, the sequences are aligned for optimal comparison purposes (e.g. gaps may be introduced in the sequence of a first amino or nucleic acid sequence for optimal alignment with a second amino or nucleic acid sequence). The amino acid residues or nucleotides at corresponding amino acid or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100).

In one embodiment, the two sequences are the same length.

In another embodiment, the two sequences are of different length and gaps are seen as different positions.

One may manually align the sequences and count the number of identical amino acids. Alternatively, alignment of two sequences for the determination of percent identity may be accomplished using a mathematical algorithm. Such an algorithm is incorporated into the NBLAST and XBLAST programs of (Altschul et al. 1997; Altschul et al. 2005).

BLAST nucleotide searches may be performed with the NBLAST program, score=100, wordlength=12, to obtain nucleotide sequences homologous to a nucleic acid molecules of the invention. BLAST protein searches may be performed with the XBLAST program, score=50, wordlength=3 to obtain amino acid sequences homologous to a protein molecule of the invention. To obtain gapped alignments for comparison purposes, Gapped BLAST may be utilised. Alternatively, PSI-Blast may be used to perform an iterated search which detects distant relationships between molecules. When utilising the NBLAST, XBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g.

“scoring matrix” and “gap penalty” may be used for alignment. In the context of the present invention, the BLASTN and PSI BLAST default settings may be advantageous. The percent identity between two sequences may be determined using techniques similar to those described above, with or without allowing gaps. In calculating percent identity, only exact matches are counted.

An embodiment of the present invention thus relates to sequences of the present invention that has some degree of sequence variation.

Several of the sequences of the present invention have been submitted to genbank: H77 (SEQ ID NOs: 1 and 12) and correspond to GenBank accession number AF009606.

The following isolates as mentioned herein are also submitted to Genbank: TNcc with GenBank accession number JX993348, J6cc with GenBank accession number JQ745650 and DBN3acc with GenBank accession number KX280714.

It should be noted that while several of the sequences in the present application (SEQ ID NOs: 7-11) are DNA sequences, the present invention contemplates the corresponding RNA sequence, and DNA and RNA complementary sequences as well. Thus, “nucleic acid molecule” is to be understood as including both DNA and RNA.

Thus, in cases where a DNA sequence is mentioned such DNA sequence refers also to the RNA equivalent i.e. with Ts exchanged with Us as well as their complimentary sequences.

In another embodiment, the HCV nucleic acid is a DNA that codes on expression or after in vitro transcription for a replication-competent HCV RNA genome, or is itself a replication-competent HCV RNA genome.

In one embodiment, the HCV nucleic acid of the invention has a full-length sequence as depicted in or corresponding to the sequences of the present invention.

In another embodiment, the HCV nucleic acid molecule according to the present invention is a fragment of SEQ ID NO: 2 or a fragment having at least 95% sequence identity with SEQ ID NO: 2. In another embodiment, the HCV nucleic acid molecule according to the present invention is a fragment of SEQ ID NO: 3 or a fragment having at least 95% sequence identity with SEQ ID NO: 3. In another embodiment, the HCV nucleic acid molecule according to the present invention is a fragment of SEQ ID NO: 4 or a fragment having at least 95% sequence identity with SEQ ID NO: 4. In another embodiment, the HCV nucleic acid molecule according to the present invention is a fragment of SEQ ID NO: 5 or a fragment having at least 95% sequence identity with SEQ ID NO: 5. In another embodiment, the HCV nucleic acid molecule according to the present invention is a fragment of SEQ ID NO: 6 or a fragment having at least 95% sequence identity with SEQ ID NO: 6.

In the present context, “fragment” is to be understood as a part of the encoded amino acid sequence or as part of the nucleic acid sequence i.e. fragments are not full-length sequences. Thus, these amino acid sequences or nucleic acid sequences are shorter than full-length amino acid sequences or nucleic acid sequences, respectively by virtue of truncation of the N-terminus or C-terminus of the amino acid sequence or both or by virtue of deletion of an internal portion or region or more internal portions or regions of the amino acid sequence or nucleic acid sequence. These fragments only comprise some of the structural or non-structural genes or part hereof. The at least 95% sequence identity is to be understood that the fragment would show at least 95% sequence identity to the corresponding fragment of SEQ ID NOs: 2, 3, 4, 5 or 6. Fragments of an amino acid sequence or a nucleic acid sequence may be generated by methods known in the art. In one embodiment, the fragment relates to the E1 and/or E2 protein.

The different regions of strain TNcc-HI is as follows: Core (1-191), E1 (192-383), E2 (384-746), p7 (747-809), NS2 (810-1026), NS3 (1027-1657), NS4A (1658-1711), NS4B (1712-1972), NS5A (1973-2420), and NS5B (2421-3011), where the numbers in the parentheses indicates amino acids according to SEQ ID NO: 4.

The different regions of strain TNcc-HI is as follows: 5′UTR (1-341), Core (342-914), E1 (915-1490), E2 (1491-2579), p7 (2580-2768), NS2 (2769-3419), NS3 (3420-5312), NS4A (5313-5474), NS4B (5475-6257), NS5A (6258-7601), NS5B (7602-9377), and 3′UTR (9378-9599) where the numbers in the parentheses indicates nucleic acids according to SEQ ID NO: 9.

The different regions of strain J6cc-HI is as follows: Core (1-191), E1 (192-383), E2 (384-750), p7 (751-813), NS2 (814-1030), NS3 (1031-1661), NS4A (1662-1715), NS4B (1716-1976), NS5A (1977-2442), and NS5B (2443-3033), where the numbers in the parentheses indicates amino acids according to SEQ ID NO: 5.

The different regions of strain J6cc-HI is as follows: 5′UTR (1-340), Core (341-913), E1 (914-1489), E2 (1490-2590), p7 (2591-2779), NS2 (2780-3430), NS3 (3431-5323), NS4A (5324-5485), NS4B (5486-6268), NS5A (6269-7666), NS5B (7667-9442), and 3′UTR (9443-9678) where the numbers in the parentheses indicates nucleic acids according to SEQ ID NO: 10.

The different regions of strain DBNcc-HI is as follows: Core (1-191), E1 (192-383), E2 (384-753), p7 (754-816), NS2 (817-1033), NS3 (1034-1664), NS4A (1665-1718), NS4B (1719-1979), NS5A (1980-2431), and NS5B (2433-3022), where the numbers in the parentheses indicates amino acids according to SEQ ID NO: 6.

The different regions of strain DBNcc-HI is as follows: 5′UTR (1-339), Core (340-912), E1 (913-1488), E2 (1489-2598), p7 (2599-2787), NS2 (2788-3438), NS3 (3439-5331), NS4A (5332-5493), NS4B (5494-6276), NS5A (6277-7632), NS5B (7633-9408), and 3′UTR (9409-9629) where the numbers in the parentheses indicates nucleic acids according to SEQ ID NO: 11.

The isolated nucleic acid molecule according to the invention may encode fragments such as the E1 and/or E2 proteins. Thus, in a further embodiment, the present invention relates to the isolated nucleic acid molecule, wherein said nucleic acid molecule encodes an amino acid sequence corresponding to E1 between amino acids 192-383 of SEQ ID NO: 4, 192-383 of SEQ ID NO: 5 or 192-383 of SEQ ID NO: 6 and/or E2 between amino acids 384-746 of SEQ ID NO: 4, 384-750 of SEQ ID NO: 5 or 384-753 of SEQ ID NO: 6.

In a further aspect, the present invention relates to an isolated nucleic acid molecule which encodes human hepatitis C virus of
genotype 1a, strain TNcc, wherein said nucleic acid molecule encodes an amino acid sequence with a sequence identity of at least 95% to that of SEQ ID NO: 25 or a fragment hereof, and wherein the amino acid sequence further comprises the following adaptive mutations V365A, F403L, N410K and V719I according to SEQ ID NO:25;
genotype 2a, strain J6cc, wherein said nucleic acid molecule encodes an amino acid sequence with a sequence identity of at least 95% to that of SEQ ID NO: 26 or a fragment hereof, and wherein the amino acid sequence further comprises the following adaptive mutations T329S, H434N and A575T according to SEQ ID NO: 26; or
genotype 3a, strain DBN3acc, wherein said nucleic acid molecule encodes an amino acid sequence with a sequence identity of at least 95% to that of SEQ ID NO: 27 or a fragment hereof, and wherein the amino acid sequence further comprises the following adaptive mutation G395R according to SEQ ID NO: 27.

In a further aspect the present invention relates to an isolated nucleic acid molecule which encodes human hepatitis C virus of
genotype 1a, strain TNcc, wherein said nucleic acid molecule encodes an amino acid sequence with a sequence identity of at least 95% to that of SEQ ID NO: 4 or a fragment hereof, and wherein the amino acid sequence comprises the following adaptive mutations V365A, F403L, N410K and V719I according to SEQ ID NO: 25;
genotype 2a, strain J6cc, wherein said nucleic acid molecule encodes an amino acid sequence with a sequence identity of at least 95% to that of SEQ ID NO: 5 or a fragment hereof, and wherein the amino acid sequence comprises the following adaptive mutations T329S, H434N and A575T according to SEQ ID NO: 26; or
genotype 3a, strain DBN3acc, wherein said nucleic acid molecule encodes an amino acid sequence with a sequence identity of at least 95% to that of SEQ ID NO: 6 or a fragment hereof, and wherein the amino acid sequence comprises the following adaptive mutations G395R according to SEQ ID NO: 27.

The molecule may encode a fragment advantageous for replicon systems. Thus, in one embodiment the present invention relates to said nucleic acid molecule, which encodes an amino acid sequence corresponding to NS3-NS5B between amino acids 1027-3011 of SEQ ID NO: 2, 1027-3011 of SEQ ID NO: 3, 1027-3011 of SEQ ID NO: 4, 1031-3033 of SEQ ID NO: 5 or 1034-3022 of SEQ ID NO: 6, or an amino acid sequence with a sequence identity of at least 95% to the amino acids 1027-3011 of SEQ ID NO: 2, 1027-3011 of SEQ ID NO: 3, 1027-3011 of SEQ ID NO: 4, 1031-3033 of SEQ ID NO: 5 or 1034-3022 of SEQ ID NO: 6.

In a further embodiment, said nucleic acid molecule encodes an amino acid sequence corresponding to NS2-NS5B between amino acids amino acids 810-3011 of SEQ ID NO: 2, 810-3011 of SEQ ID NO: 3, 810-3011 of SEQ ID NO: 4, 814-3033 of SEQ ID NO: 5 or 817-3022 of SEQ ID NO: 6, or an amino acid sequence with a sequence identity of at least 95% to the amino acids 810-3011 of SEQ ID NO: 2, 810-3011 of SEQ ID NO: 3, 810-3011 of SEQ ID NO: 4, 814-3033 of SEQ ID NO: 5 or 817-3022 of SEQ ID NO: 6.

In a still further embodiment, said nucleic acid molecule further comprises a 5′UTR region, such as the 5′UTR region between nucleic acids 1-341 of SEQ ID NO: 7, 1-341 of SEQ ID NO: 8, 1-341 of SEQ ID NO: 9, 1-340 of SEQ ID NO: 10 or 1-339 of SEQ ID NO: 11, or a nucleic acid sequence with a sequence identity of at least 95% to nucleic acids 1-341 of SEQ ID NO: 7, 1-341 of SEQ ID NO: 8, 1-341 of SEQ ID NO: 9, 1-340 of SEQ ID NO: 10 or 1-339 of SEQ ID NO: 11.

In an even further embodiment, said nucleic acid molecule further comprises a 3′UTR region, such as the 3′UTR region between nucleic acids 9378-9599 of SEQ ID NO: 7, 9378-9599 of SEQ ID NO: 8, 9378-9599 of SEQ ID NO: 9, 9443-9678 of SEQ ID NO: 10 or 9409-9629 of SEQ ID NO: 11, or a nucleic acid sequence with a sequence identity of at least 95% to nucleic acids 9378-9599 of SEQ ID NO: 7, 9378-9599 of SEQ ID NO: 8, 9378-9599 of SEQ ID NO: 9, 9443-9678 of SEQ ID NO: 10 or 9409-9629 of SEQ ID NO: 11.

Various modifications for example of the 5′ and 3′ UTR are also contemplated by the invention.

In one embodiment, the nucleic acid molecule comprises the sequence encoding the genes NS2-NS5B or part hereof as disclosed above and a 5′UTR region such as a 5′UTR region as disclosed herein. In a further embodiment, the nucleic acid molecule comprises the sequence encoding the genes NS2-NS5B or part hereof as disclosed above and a 3′UTR region such as a 3′UTR region as disclosed herein. In a still further embodiment, the nucleic acid molecule comprises the sequence encoding the genes NS2-NS5B or part hereof as disclosed above, a 3′UTR region and a 5′UTR region such as a 5′UTR region and 3′UTR region as disclosed herein.

In one embodiment, the nucleic acid molecule comprises the sequence encoding the genes NS3-NS5B or part hereof as disclosed above and a 5′UTR region such as a 5′UTR region as disclosed herein. In a further embodiment, the nucleic acid molecule comprises the sequence encoding the genes NS3-NS5B or part hereof as disclosed above and a 3′UTR region such as a 3′UTR region as disclosed herein. In a still further embodiment, the nucleic acid molecule comprises the sequence encoding the genes NS3-NS5B or part hereof as disclosed above, a 3′UTR region and a 5′UTR region such as a 5′UTR region and a 3′UTR region as disclosed herein.

In another embodiment, the nucleic acid further comprises a reporter gene, which, in one embodiment, is a gene encoding neomycin phosphotransferase, Renilla luciferase, secreted alkaline phosphatase (SEAP), Gaussia luciferase or the green fluorescent protein.

Naturally, as noted above, the HCV nucleic acid sequence of the invention is selected from the group consisting of double stranded DNA, positive-sense cDNA, or negative-sense cDNA, or positive-sense RNA or negative-sense RNA or double stranded RNA.

Thus, where particular sequences of nucleic acids of the invention are set forth, both DNA and corresponding RNA are intended, including positive and negative strands thereof.

In a further embodiment, the nucleic acid sequences or the nucleic acid sequences with any mutation described in this document are obtained by any other means than what is described above.

Nucleic acid molecules according to the present invention may be inserted in a plasmid vector for transcription of the corresponding HCV RNA. Thus, the HCV DNA may comprise a promoter 5′ of the 5′-UTR on positive-sense DNA, whereby transcription of template DNA from the promoter produces replication-competent RNA. The promoter can be selected from the group consisting of a eukaryotic promoter, yeast promoter, plant promoter, bacterial promoter, or viral promoter.

Thus, in one embodiment the present invention provides a cassette vector for cloning viral genomes, comprising, inserted therein, the nucleic acid sequence according to the invention and having an active promoter upstream thereof.

### Adaptive Mutations

Adapted mutants of a HCV-cDNA construct or HCV-RNA full-length genome with improved abilities to generate infectious viral particles in cell culture compared to the original HCV-cDNA construct or the original HCV-RNA full-length genome are characterized in that they are obtainable by a method in which the type and number of mutations in a cell culture adapted HCV-RNA genome are determined through sequence analysis and sequence comparison and these mutations are introduced into a HCV-cDNA construct, particularly a HCV-cDNA construct according to the present invention, or into an (isolated) HCV-RNA full-length genome, either by site-directed mutagenesis, or by exchange of DNA fragments containing the relevant mutations.

The present inventors here report adaptive mutations, which allow efficient formation and release of viral particles in cell culture in a high-yield, and thus the present invention relates to these adaptive mutations in the present use as well as use in other strains by changing equivalent positions of such genomes to the adapted nucleotide or amino acid described.

A group of preferred HCV-cDNA constructs, HCV-RNA full-length genomes with the ability to release viral particles in cell culture, which are consequently highly suitable for practical use, is characterized in that it contains one, several or all of the nucleic acid exchanges listed herein and/or one or several or all of the following amino acid exchanges.

Another group of preferred HCV-cDNA constructs, HCV-RNA replication genomes with the ability to replicate in cell culture, which are consequently highly suitable for practical use, is characterized in that it contains one, several or all of the nucleic acid exchanges listed herein and/or one or several or all of the following amino acid exchanges.

One embodiment of the present invention relates to adaptive mutations, wherein the adaptive mutation is a mutation that can be observed by clonal or direct sequencing of recovered replicating genomes of the sequences of the present invention.

Thus in a further embodiment, the present invention relates to nucleic acid molecules according to the present invention, wherein said molecule comprises one or more adaptive mutations in Core, E1, E2, p7, NS2, NS3, NS4A, NS4B, NS5A or NS5B singly or in combination.

In the context of the present invention the term “adaptive mutation” is meant to cover mutations identified in passaged viruses that provide the original and any other HCV sequence the ability to grow efficiently in culture. Furthermore, all introductions of mutations into the sequences described, whether or not yielding better growth abilities, and the introduction of these mutations into any HCV sequence should be considered.

Thus the described mutations enable the HCV-RNA genome (e.g. derived from a HCV-cDNA clone) to form viral particles in and release these from suitable cell lines. In addition some of the described mutations might change the function of the concerned proteins in favourable ways, which might be exploited in other experimental systems employing these proteins.

This also includes other HCV genomes with adaptive mutations, all of them, combinations of them or individual mutations that grow in culture.

It should be understood that any feature and/or aspect discussed above in connection with the mutations according to the invention applies by analogy to both single mutations and any combination of the mutations.

In another embodiment all the amino acid changes observed herein are provided by the present application. The skilled addressee can easily obtain the same amino acid change by mutating another base of the codon and hence all means of obtaining the given amino acid sequence is intended. In one embodiment, the adaptive mutation may be described according to the amino acid sequence and the mutation/change in amino acid observed i.e. the substitution of one amino acid with another.

### Titer

To determine the efficiency of the developed system, HCV RNA titers may be determined in IU/ml (international units/ml) with Taq-Man Real-Time-PCR and infectious titers may be determined with a focus forming unit assay.

The infectious titers are determined as TCID50/ml (median tissue culture infectious dose/ml) or FFU/ml (focus forming unites/ml); in such method, infectivity titers are determined by infection of cell culture replicates with serial dilutions of virus containing supernatants and, following immuno-stainings for HCV antigens, counting of HCV-antigen positive cell foci.

HCV RNA titers and infectivity titers can be determined extracellularly, in cell culture supernatant (given as IU and TCID50 or FFU per ml, respectively) or intracellularly, in lysates of pelleted cells (given as IU and TCID50 or FFU related to the given cell number or culture plate wells, which was lysed).

In one embodiment, said molecule is capable of generating an HCV infectivity titer of 2 log 10 FFU/ml [(focus forming unites)/ml] or above following transfection and/or subsequent viral passage.

In one embodiment, said molecule is capable of generating an HCV infectivity titer of 2 log 10 FFU/ml or above, such as an HCV infectivity titer of 3 log 10 FFU/ml or above, like an HCV infectivity titer of 4 log 10 FFU/ml or above, such as an HCV infectivity titer of 5 log 10 FFU/ml or above, like an HCV infectivity titer of 6 log 10 FFU/ml or above, such as an HCV infectivity titer of 7 log 10 FFU/ml or above following transfection and/or subsequent viral passage.

In another embodiment, the present invention relates to a nucleic acid molecule according to the invention, wherein said molecule is capable of generating an HCV infectivity titer of at least 102 FFU/ml or above following transfection and/or subsequent viral passage, such as a titer of at least 103 FFU/ml, such as a titer of at least 10⁴ FFU/ml, such as a titer of at least 105 FFU/ml, such as a titer of at least 10⁶ FFU/ml, such as a titer of at least 107 FFU/ml.

It is of course evident to the skilled addressee that the titers described here are obtained using the assay described in this text. Any similar or equivalent titer determined by any method is thus evidently within the scope of the present invention.

### Compositions

One embodiment of the present invention relates to a composition comprising a nucleic acid molecule according to the invention suspended in a suitable amount of a pharmaceutical acceptable diluent or excipient.

In another embodiment, this invention provides for compositions comprising an isolated nucleic acid, vector or cell of this invention, or an isolated nucleic acid obtained via the methods of this invention.

In one embodiment, the term “composition” refers to any such composition suitable for administration to a subject, and such compositions may comprise a pharmaceutically acceptable carrier or diluent, for any of the indications or modes of administration as described. The active materials in the compositions of this invention can be administered by any appropriate route, for example, orally, parenterally, intravenously, intradermally, subcutaneously, or topically, in liquid or solid form. It is to be understood that any applicable drug delivery system may be used with the compositions and/or agents/vectors/cells/nucleic acids of this invention, for administration to a subject, and is to be considered as part of this invention.

The compositions of the invention can be administered as conventional HCV therapeutics. The compositions of the invention may include more than one active ingredient which interrupts or otherwise alters groove formation, or occupancy by RNA or other cellular host factors, in one embodiment, or replicase components, in another embodiment, or zinc incorporation, in another embodiment.

The precise formulations and modes of administration of the compositions of the invention will depend on the nature of the anti-HCV agent, the condition of the subject, and the judgment of the practitioner. Design of such administration and formulation is routine optimization generally carried out without difficulty by the practitioner.

It is to be understood that any of the methods of this invention, whereby a nucleic acid, vector or cell of this invention is used, may also employ a composition comprising the same as herein described, and is to be considered as part of this invention.

“Pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

The term “excipient” refers to a diluent, adjuvant, carrier, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The term “adjuvant” refers to a compound or mixture that enhances the immune response to an antigen. An adjuvant can serve as a tissue depot that slowly releases the antigen and also as a lymphoid system activator that non-specifically enhances the immune response. Often, a primary challenge with an antigen alone, in the absence of an adjuvant, will fail to elicit a humoral or cellular immune response. Adjuvants include, but are not limited to, MF-59, CAF adjuvants, CpG adjvuants, complete Freund's adjuvant, incomplete Freund's adjuvant, saponin, mineral gels such as aluminum hydroxide, surface active substances such as lysolecithin, pluronicpolyols, polyanions, peptides, oil or hydrocarbon emulsions, keyhole limpet hemocyanins, dinitrophenol, and potentially useful human adjuvants such as BCG (bacilleCalmette-Guerin) and Corynebacterium parvum.

Preferably, the adjuvant is pharmaceutically acceptable.

Thus, one embodiment of the present invention relates to a composition comprising a nucleic acid molecule according to the present invention suspended in a suitable amount of a pharmaceutical acceptable diluent or excipient.

### Cells

The nucleotides of the present invention may be used to provide a method for identifying additional cell lines that are permissive for infection with HCV, comprising contacting (e.g. transfecting) a cell line in tissue culture with an infectious amount of HCV RNA of the present invention, e.g., as produced from the plasmid clones, and detecting replication and/or formation and release of viral particles of HCV in cells of the cell line.

Naturally, the invention extends as well to a method for identifying an animal that is permissive for infection with HCV, comprising introducing an infectious amount of the HCV RNA, e.g., as produced by the plasmids, to the animal, and detecting replication and/or formation and release of viral particles of HCV in the animal. By providing infectious HCV, e.g. comprising a dominant selectable marker, the invention further provides a method for selecting for HCV with further adaptive mutations that permit higher levels of HCV replication in a permissive cell line or animal comprising contacting (e.g. transfecting) a cell line in culture, or introducing into an animal, an infectious amount of the HCV RNA, and detecting progressively increasing levels of HCV RNA and infectious HCV viral particles in the cell line or the animal.

In a specific embodiment, the adaptive mutation permits modification of HCV tropism. An immediate implication of this aspect of the invention is creation of new valid cell culture and animal models for HCV infection.

The permissive cell lines or animals that are identified using the nucleic acids of the invention are very useful, inter alia, for studying the natural history of HCV infection, isolating functional components of HCV, and for sensitive, fast diagnostic applications, in addition to producing authentic HCV virus or components thereof.

Because the HCV DNA, e.g., plasmid vectors, of the invention encode HCV components, expression of such vectors in a host cell line transfected, transformed, or transduced with the HCV DNA can be effected.

For example, a baculovirus or plant expression system can be used to express HCV virus particles or components thereof. Thus, a host cell line may be selected from the group consisting of a bacterial cell, a yeast cell, a plant cell, an insect cell, and a mammalian cell.

In one embodiment, the cell is a hepatocyte, or in another embodiment, the cell is the Huh-7 hepatoma cell line or a derived cell line such as Huh7.5 or Huh7.5.1 cell line.

In one embodiment, the cell, or in another embodiment, cell systems of this invention comprise primary cultures or other, also non hepatic cell lines. “Primary cultures” refers, in one embodiment, to a culture of cells that is directly derived from cells or tissues from an individual or an animal, as well as cells derived by passage from these cells, or immortalized cells.

In one embodiment, “cell line” refers to a population of cells capable of continuous or prolonged growth and division in vitro. The term “cell lines” also includes immortalized cells. Often, cell lines are clonal populations derived from a single progenitor cell. Such cell lines are also termed “cell clones”. It is further known in the art that spontaneous or induced changes can occur in karyotype during storage or transfer of such clonal populations. Therefore, cells derived from the cell clones referred to may not be precisely identical to the ancestral cells or cultures. According to the present invention, such cell clones may be capable of supporting replication of a vector, virus, viral particle, etc., of this invention, without a significant decrease in their growth properties, and are to be considered as part of this invention.

It is to be understood that any cell of any organism that is susceptible to infection by or propagation of an HCV construct, virus or viral particle of this invention is to be considered as part of this invention, and may be used in any method of this invention, such as for screening or other assays, as described herein.

Thus, one embodiment of the present invention relates to a cell comprising the nucleic acid according to the present invention, the composition of present invention or the cassette vector of the present invention.

Another embodiment of the present invention relates to a method for producing a cell, which replicates human hepatitis C virus and optionally produces a virus particle comprising introducing a nucleic acid molecule of the present invention into a cell. In a further embodiment, the present invention relates to a method for producing a cell, which replicates human hepatitis C virus and optionally, produces a virus particle comprising introducing a nucleic acid molecule of the present invention into a cell.

Another embodiment of the present invention relates to a cell obtainable by the methods of the present invention.

Also, a method for in vitro producing a hepatitis C virus-infected cell is described comprising culturing the cell which produces virus particles of the present invention and infecting other cells with the produced virus particle in the culture. Another embodiment relates to a method for in vitro producing a hepatitis C virus-infected cell is described comprising culturing a cell and infecting other cells with the produced virus particle in the culture.

Naturally, the invention extends to any cell obtainable by such methods, for example any in vitro cell line infected with HCV, wherein the HCV has a genomic RNA sequence as described herein such as a hepatitis C virus infected cell obtainable by any of the methods described.

In one embodiment, the cell line is a hepatocyte cell line such as Huh7 or derived cell lines e.g. Huh7.5 or Huh7.5.1. In another embodiment, the cell is Huh7.5.

In another embodiment the cell is any cell expressing the genes necessary for HCV infection and replication, such as but not limited to CD81, SR-BI,

Claudin-1, -4, -6 or -9, Occludin, and the low-density lipid receptor.

The invention further provides various methods for producing HCV virus particles, including isolating HCV virus particles from the HCV-infected non-human animal; culturing a cell line of the invention under conditions that permit HCV replication and virus particle formation; or culturing a host expression cell line transfected with HCV DNA under conditions that permit expression of HCV particle proteins; and isolating HCV particles or particle proteins from the cell culture. The present invention extends to an HCV virus particle comprising a replication-competent HCV genome RNA, or a replication-defective HCV genome RNA, corresponding to an HCV nucleic acid of the invention as well.

A further aspect of the present invention relates to a method for producing a hepatitis C virus replication system, comprising culturing a cell according to the present invention to allow the cell to replicate the virus genome.

HCV replication systems using sub-genomic or full-length genomes have been valuable and useful tools for development and preclinical testing of drugs targeting HCV replication. These models provide fundamental tools for testing of drug efficacy in the context of viral replication, and the infectious genotype 1a, 2a and 3a genomes developed here can be applied to develop such systems.

In one embodiment, the cell being cultured for the replication system only comprises a fragment of the amino acid sequence allowing the HCV to replicate but not to form viruses.

### Virus Particle

The production of authentic virus proteins (antigens) may be used for the development and/or evaluation of diagnostics. The cell culture system according to the invention also allows the expression of HCV antigens in cell cultures. In principle these antigens can be used as the basis for diagnostic detection methods.

The production of HCV viruses and virus-like particles, in particular for the development or production of therapeutics and vaccines as well as for diagnostic purposes is an embodiment of the present invention. Especially cell culture adapted complete HCV genomes, which could be produced by using the cell culture system according to the invention, are able to replicate and form viral particles in cell culture with high efficiency. These genomes have the complete functions of HCV and in consequence they are able to produce infectious viruses.

Thus in one embodiment the present invention relates to a method for producing a hepatitis C virus particle of the present invention or parts thereof, comprising culturing a cell or an animal to allow either to produce the virus. In a further embodiment, the present invention relates to a method for producing a hepatitis C virus particle of the present invention comprising culturing a cell to allow to produce the virus.

In a preferred embodiment, the cell is a Huh7.5 cell.

In another embodiment, the invention provides a hepatitis C virus particle obtainable by the method described.

In a further embodiment, the invention provides a mixture of hepatitis C virus particles as described herein comprising hepatitis C virus particles of at least two genotypes selected from the group consisting of genotype 1a, genotype 2a and genotype 3a.

In an even further embodiment, the invention relates to a method for producing a hepatitis C virus particle, comprising culturing a cell as described herein to allow the cell to produce the virus.

Because the invention provides, inter alia, infectious HCV RNA, the invention provides a method for infecting an animal with HCV, which comprises administering an infectious dose of HCV RNA, such as the HCV RNA transcribed from the plasmids described above, to the animal. Naturally, the invention provides a non-human animal infected with HCV of the invention, which non-human animal can be prepared by the foregoing methods.

In one embodiment the introduced mutations attenuate the virus in vivo.

A further advantage of the present invention is that, by providing a complete functional HCV genome, authentic HCV viral particles or components thereof, which may be produced with native HCV proteins or RNA in a way that is not possible in subunit expression systems, can be prepared.

In addition, since each component of HCV of the invention is functional (thus yielding the authentic HCV), any specific HCV component is an authentic component, i.e., lacking any errors that may, at least in part, affect the clones of the prior art. Indeed, a further advantage of the invention is the ability to generate HCV virus particles or virus particle proteins that are structurally identical to or closely related to natural HCV virions or proteins. Thus, in a further embodiment, the invention provides a method for propagating HCV in vitro comprising culturing a cell line contacted with an infectious amount of HCV RNA of the invention, e.g., HCV RNA transcribed from the plasmids described above, under conditions that permit replication of the HCV RNA.

In one embodiment, the method further comprises isolating infectious HCV. In another embodiment, the method further comprises freezing aliquots of said infectious HCV. According to this aspect of the invention, and in one embodiment, the HCV is infectious following thawing of said aliquots, and in another embodiment, the HCV is infectious following repeated freeze-thaw cycles of said aliquots.

A further embodiment of the present invention relates to a method for in vitro producing a hepatitis C virus-infected cell comprising culturing a cell according to the present invention and infecting other cells with the produced virus particle in the culture.

### Screening for Anti-Viral Drugs and the Determination of Drug Resistance.

It can be assumed that resistance to therapy occurs due to the high mutation rate of the HCV genome. This resistance, which is very important for the clinical approval of a substance, can be detected with the cell culture system according to the invention. Cell lines, in which the HCV-RNA construct or the HCV genome or subgenome replicates and produces infectious viral particles, are incubated with increasing concentrations of the relevant substance and the replication of the viral RNA is either determined by means of an introduced reporter gene or through the qualitative or quantitative detection of the viral nucleic acids or proteins. The release of viral particles is determined by measuring HCV RNA and infectivity titers in the cell culture supernatant. Alternatively, the number of antigen-expressing cells is determined. Resistance is given if no or a reduced inhibition of the replication and release of viral particles can be observed with the normal concentration of the active substance. The nucleotide and amino acid replacements responsible for the therapy resistance can be determined by cloning the HCV-RNA (for example by the means of RT-PCR) and sequence analysis. By cloning the relevant replacement(s) into the original construct its causality for the resistance to therapy can be proven.

The systems developed in this invention can be used for specific testing of therapeutics in general and therapeutics targeting viral entry, assembly and release.

Genomes with the sequences of the present invention are valuable for testing of neutralizing antibodies and other drugs acting on entry level, such as fusion inhibitors.

In one embodiment the present invention relates to a method for identifying neutralizing antibodies.

In another embodiment, the present invention relates to a method for identifying cross-genotype neutralizing antibodies.

In one embodiment the present invention relates to a method for screening new HCV genotype 1a, 2a and/or 3a inhibitors or neutralizing antibodies, comprising
a) culturing at least one selected from the group consisting of a cell according to the present invention, a hepatitis C virus infected cell according to the present invention and a hepatitis C virus particle obtainable by the present invention together with a hepatitis C virus permissive cell, and
b) subjecting said virus or virus infected cell culture to a blood sample or derivatives thereof from an HCV genotype 1a, 2a and/or 3a infected patient
c) detecting the amount of replicating RNA and/or the virus particles.

Inhibitors targeting the HCV non-structural proteins NS3/4A, NS5A and NS5B have been developed and are successfully used in the clinic. The present invention offers novel culture systems where additional HCV isolates can be tested to generate efficient cross-reactive inhibitors.

The p7 peptide features two transmembrane domains (TM1 and TM2), and p7 monomers multimerize to form a putative ion channel. Additionally p7 has been shown to contain genotype specific sequences required for genotype specific interactions between p7 and other HCV proteins. Hence, new compounds targeting the putative p7 ion-channel and autoprotease inhibitors interfering with NS2, or drugs targeting the viral NS3 helicase region, and drugs targeting cellular proteins involved in the described processes can be tested.

Thus, one embodiment of the present invention relates to a method for screening an anti-hepatitis C virus substance, comprising
a) culturing at least one selected from the group consisting of a cell according to the present invention, a hepatitis C virus infected cell according to the present invention and a hepatitis C virus particle obtainable by the present invention together with a hepatitis C virus permissive cell,
b) subjecting said virus or virus infected cell culture to the anti-hepatitis C virus substance, and
c) detecting the replicating RNA and/or the virus particles in the resulting culture.

Another embodiment of the present invention relates to a method for screening an anti-hepatitis C virus substance, comprising
a) culturing at least one selected from the group consisting of a cell comprising a nucleic acid molecule according to the present invention, a cell as described herein, a hepatitis C virus particle obtainable from a method as described herein and a hepatitis C virus replication obtainable from a method as described herein together with a hepatitis C virus permissive cell, and
b) detecting the replicating RNA or the virus particles in the resulting culture.

In another embodiment, the inhibition of HCV replication and/or infection and/or pathogenesis includes inhibition of downstream effects of HCV. In one embodiment, downstream effects include neoplastic disease, including, in one embodiment, the development of hepatocellular carcinoma.

In one embodiment, the invention provides a method of screening for anti-HCV therapeutics, the method comprising contacting a cell with an isolated nucleic acid molecule encoding an infectious recombinant HCV genome, comprising a chimeric HCV genome or a replicating subunit and contacting the cell with a candidate molecule, independently contacting the cell with a placebo and determining the effects of the candidate molecule on HCV infection, replication, or cell-to-cell spread, versus the effects of the placebo, wherein a decrease in the level of HCV infection, replication, or cell-to-cell spread indicates the candidate molecule is an anti-HCV therapeutic.

In one embodiment, the method may be conducted in vitro or in vivo. In one embodiment, the cells as described may be in an animal model, or a human subject, entered in a clinical trial to evaluate the efficacy of a candidate molecule. In one embodiment, the molecule is labelled for easier detection, including radio-labelled, antibody labelled for fluorescently labelled molecules, which may be detected by any means well known to one skilled in the art.

In one embodiment, the candidate molecule is an antibody.

In another embodiment, the candidate molecule is a small molecule. In one embodiment, the phrase “small molecule” refers to, inter-alia, synthetic organic structures typical of pharmaceuticals, peptides, nucleic acids, peptide nucleic acids, carbohydrates, lipids, and others, as will be appreciated by one skilled in the art. In another embodiment, small molecules, may refer to chemically synthesized peptidomimetics of the 6-mer to 9-mer peptides of the invention.

In another embodiment, the candidate molecule is a nucleic acid. Numerous nucleic acid molecules can be envisioned for use in such applications, including antisense, siRNA, ribozymes, etc., as will be appreciated by one skilled in the art.

It is to be understood that the candidate molecule identified and/or evaluated by the methods of this invention, may be any compound, including, inter-alia, a crystal, protein, peptide or nucleic acid, and may comprise an HCV viral product or derivative thereof, of a cellular product or derivative thereof. The candidate molecule in other embodiments may be isolated, generated synthetically, obtained via translation of sequences subjected to any mutagenesis technique, or obtained via protein evolution techniques, well known to those skilled in the art, each of which represents an embodiment of this invention, and may be used in the methods of this invention, as well.

In one embodiment, the compound identified in the screening methods as described, may be identified by computer modelling techniques, and others, as described herein. Verification of the activity of these compounds may be accomplished by the methods described herein, where, in one embodiment, the test compound demonstrably affects HCV infection, replication and/or pathogenesis in an assay, as described. In one embodiment, the assay is a cell-based assay, which, in one embodiment, makes use of primary isolates, or in another embodiment, cell lines, etc. In one embodiment, the cell is within a homogenate, or in another embodiment, a tissue slice, or in another embodiment, an organ culture. In one embodiment, the cell or tissue is hepatic in origin, or is a derivative thereof. In another embodiment, the cell is a commonly used mammalian cell line, which has been engineered to express key molecules known to be, or in another embodiment, thought to be involved in HCV infection, replication and/or pathogenesis.

In another embodiment, protein, or in another embodiment, peptide or in another embodiment, other inhibitors of the present invention cause inhibition of infection, replication, or pathogenesis of HCV in vitro or, in another embodiment, in vivo when introduced into a host cell containing the virus, and may exhibit, in another embodiment, an EC50 in the range of from about 0.0001 nM to 100 UM in an in vitro assay for at least one step in infection, replication, or pathogenesis of HCV, more preferably from about 0.0001 nM to 75 μM, more preferably from about 0.0001 nM to 50 UM, more preferably from about 0.0001 nM to 25 UM, more preferably from about 0.0001 nM to 10 UM, and even more preferably from about 0.0001 nM to 1 μM.

In another embodiment, the inhibitors of HCV infection, or in another embodiment, replication, or in another embodiment, pathogenesis, may be used, in another embodiment, in ex vivo scenarios, such as, for example, in routine treatment of blood products wherein a possibility of HCV infection exists, when serology shows a lack of HCV infection.

In another embodiment, the anti-HCV therapeutic compounds identified via any of the methods of the present invention can be further characterized using secondary screens in cell cultures and/or susceptible animal models. In one embodiment, a small animal model may be used, such as, for example, urokinase-type plasminogen activator-severe combined immunodeficiency (uPA-SCID) mice with human liver xenografts (human liver chimeric mice) or a tree shrew Tupaia belangeri chinensis. In another embodiment, an animal model may make use of a chimpanzee. Test animals may be treated with the candidate compounds that produced the strongest inhibitory effects in any of the assays/methods of this invention. In another embodiment, the animal models provide a platform for pharmacokinetic and toxicology studies.

### Vaccines

The construct according to the invention by itself can also be used for various purposes in all its embodiments. This includes the construction of hepatitis C viruses or HCV-like particles and their production in cell cultures as described.

HCV or HCV-like particles, as well as encoding nucleic acid molecules or deduced peptides or expressed recombinant proteins, can be used in particular as vaccine. Thus, one embodiment of the present invention relates to a hepatitis C virus vaccine comprising a nucleic acid molecule, the encoded amino acid sequence or a fragment hereof as described herein, a hepatitis C virus particle as described herein, or a mixture of hepatitis C virus particles as described herein. A further embodiment of the present invention relates to a hepatitis C virus vaccine comprising a mixture of nucleic acid molecules, encoded amino acid sequences and/or HCV or HCV-like particles as described herein.

In a further embodiment, the hepatitis C virus vaccine according to the present invention comprises nucleic acid molecules, encoded amino acid sequences and/or HCV or HCV-like particles of at least two genotypes selected from the group consisting of genotype 1a, genotype 2a and genotype 3a.

In an even further embodiment, the vaccine is one or more selected from the group consisting of an inactivated whole virus vaccine, an attenuated whole virus vaccine, a recombinant protein-based vaccine, a virus-like particle based vaccine, a DNA vaccine, an RNA vaccine, and a viral vector based vaccine. In yet another embodiment, the hepatitis C virus vaccine is a DNA vaccine, such as a viral vector based vaccine comprising DNA. In yet another embodiment, the hepatitis C virus vaccine is an RNA vaccine.

The vaccine is preferably suitable for administration to a subject, and may comprise a pharmaceutically acceptable carrier or diluent, for any of the indications or modes of administration as described. The active materials in the vaccine can be administered by any appropriate route, for example, orally, parenterally, intravenously, intradermally, subcutaneously, or topically, in liquid or solid form.

The hepatitis C virus vaccine may further comprise an excipient, for example an adjuvant, as described herein. Thus, in a further embodiment, the hepatitis C virus vaccine further comprises an excipient, such as an adjuvant.

In another embodiment, the nucleic acids, vectors, viruses, or viral particles may be further engineered to express a heterologous protein, which, in another embodiment, is mammalian or a derivative thereof, which is useful in combating HCV infection or disease progression. Such proteins may comprise cytokines, growth factors, tumor suppressors, or in one embodiment, may following infection, be expressed predominantly or exclusively on an infected cell surface. According to this aspect of the invention, and in one embodiment, such molecules may include costimulatory molecules, which may serve to enhance immune responses to infected cells, or preneoplastic cells, or neoplastic cells, which may have become preneoplastic or neoplastic as a result of HCV infection. In one embodiment, the heterologous sequence encoded in the nucleic acids, vectors, viruses, or viral particles of this invention may be involved in enhanced uptake of a nucleic acids, vectors, viruses, or viral particles, and may specifically target receptors thought to mediate HCV infection.

Further, the present invention relates to a method for producing a hepatitis C virus vaccine comprising using a nucleic acid, the encoded amino acid or a fragment hereof as described herein, a hepatitis C virus particle obtained as described herein, or a mixture of hepatitis C virus particles obtained as described herein or a mixture of one or more nucleic acid molecules, encoded amino acid sequences and/or HCV or HCV-like particles as described herein as an antigen.

In a further aspect, the present invention relates to use of a nucleic acid molecule or the encoded amino acid sequence or a fragment hereof as described herein; a hepatitis C virus particle as described herein or a part thereof; a mixture of hepatitis C virus particles as described herein; or a mixture of one or more hereof; or a hepatitis C virus vaccine as described herein for raising neutralizing antibodies. In one embodiment the present invention relates to a method of raising neutralizing antibodies. In a further embodiment, the present invention relates to the use of a hepatitis C virus vaccine for raising neutralizing antibodies.

In a further aspect, the present invention relates to use of a nucleic acid molecule or the encoded amino acid sequence or a fragment hereof as described herein; a hepatitis C virus particle as described herein or a part thereof; a mixture of hepatitis C virus particles as described herein; or a mixture of one or more hereof; or a hepatitis C virus vaccine as described herein for raising cross-neutralizing antibodies. In another embodiment the present invention relates to a method of raising cross-neutralizing antibodies. In a further embodiment, the present invention relates to the use of a hepatitis C virus vaccine for raising cross-neutralizing antibodies.

### Antibodies

Another aspect of the present invention relates to an antibody against a nucleic acid molecule or the encoded amino acid sequence or a fragment hereof as described herein; a hepatitis C virus particle as described herein or a part thereof; or a mixture of hepatitis C virus particles as described herein; or a mixture of one or more hereof. In one embodiment, the present invention relates to an antibody against the hepatitis C virus particle.

In one embodiment, the term “antibody” refers to intact molecules as well as functional fragments thereof, such as Fab, F(ab′)2, and Fv. In one embodiment, the term “Fab” refers to a fragment, which contains a monovalent antigen-binding fragment of an antibody molecule, and in one embodiment, can be produced by digestion of whole antibody with the enzyme papain to yield an intact light chain and a portion of one heavy chain, or in another embodiment can be obtained by treating whole antibody with pepsin, followed by reduction, to yield an intact light chain and a portion of the heavy chain. In one embodiment, the term “F(ab′)2”, refers to the fragment of the antibody that can be obtained by treating whole antibody with the enzyme pepsin without subsequent reduction, F(ab′)2 is a dimer of two Fab′ fragments held together by two disulfide bonds. In another embodiment, the term “Fv” refers to a genetically engineered fragment containing the variable region of the light chain and the variable region of the heavy chain expressed as two chains, and in another embodiment, the term “single chain antibody” or “SCA” refers to a genetically engineered molecule containing the variable region of the light chain and the variable region of the heavy chain, linked by a suitable polypeptide linker as a genetically fused single chain molecule. Also included are chimeric antibodies, for example, monoclonal antibodies or fragments thereof. Further included are camelid antibodies or nanobodies.

Methods of producing these functional fragments, chimeric antibodies, camelid antibodies or nanobodies are known in the art.

In a further embodiment, an antibody or a mixture of antibodies according to the present invention may exhibit an EC50 in the range of from about 0.0001-1000 μg/ml, such as 0.001-900 μg/ml, such as 0.01-800 μg/ml, such as 0.1-700 μg/ml, such as 1-600 μg/ml, such as 10-500 μg/ml, like 25-250 μg/ml, such as 40-150 μg/ml for neutralization of same genotype.

In a further embodiment, an antibody or a mixture of antibodies according to the present invention may exhibit an EC50 in the range of from about 0.0001-1000 μg/ml, such as 0.001-900 μg/ml, such as 0.01-800 μg/ml, such as 0.1-700 μg/ml, such as 1-600 μg/ml, such as 10-500 μg/ml, like 25-250 μg/ml, such as 50-100 μg/ml for neutralization of different genotype.

### Medical Uses

The antigen, hepatitis C virus particle as described herein, a mixture of hepatitis C virus particles as described herein, a hepatitis C virus vaccine as described herein or a hepatitis C virus vaccine obtained by the method as described herein may be used for vaccination of a subject against hepatitis C virus. Hence, in one aspect of the present invention relates to a hepatitis C virus particle as described herein, a mixture of hepatitis C virus particles as described herein, a hepatitis C virus vaccine as described herein or a hepatitis C virus vaccine obtained by the method as described herein for use in vaccination of a subject against hepatitis C virus.

In one embodiment, the hepatitis C virus is a hepatitis C virus of genotype 1-6. In a further embodiment, the hepatitis C virus is a hepatitis C virus of genotype 1a, 1b, 2a, 2b, 3a, 4a, 5a, and/or 6a.

In a further aspect, the present invention relates to an antibody as described herein, a hepatitis C virus particle as described herein, a mixture of hepatitis C virus particles as described herein, a hepatitis C virus vaccine as described herein or a hepatitis C virus vaccine obtained by the method as described herein for use as a medicament.

The antibody as described herein, the hepatitis C virus particle as described herein, a mixture of hepatitis C virus particles as described herein, a hepatitis C virus vaccine as described herein or a hepatitis C virus vaccine obtained by the method as described herein may be used for treating, alleviating or preventing a hepatitis C virus infection. Hence in an even further aspect, the present invention relates to an antibody as described herein, a hepatitis C virus particle as described herein, a mixture of hepatitis C virus particles as described herein, a hepatitis C virus vaccine as described herein or a hepatitis C virus vaccine obtained by the method as described herein for use in treating, alleviating or preventing a hepatitis C virus infection.

In one embodiment, the hepatitis C virus infection is caused by a hepatitis C virus of a genotype selected from the group consisting of genotype 1, genotype 2, genotype 3, genotype 4, genotype 5 and genotype 6. In a further embodiment, the hepatitis C virus infection is caused by a hepatitis C virus of a genotype selected from the group consisting of genotype 1a, genotype 1b, genotype 2a, genotype 2b, genotype 3a, genotype 4a, genotype 5a, and genotype 6a.

### Uses

The cell culture system developed of the present invention will be a valuable tool to address different research topics.

It will allow the isolate, subtype and genotype specific study of functions of all HCV genome regions and proteins using reverse genetics.

Accordingly, the developed cell culture systems allow individual patient targeting. This means that when a new potential therapeutic candidate is discovered it is possible to test this particular candidate or combination of candidates on novel HCV isolates grown in culture.

Knowing which specific genotype the candidate is functioning towards, it allows an individual treatment of each patient dependent on which specific genotype the patient is infected with. Furthermore, these cell culture systems allow the development of antibodies and vaccines targeting individual patients.

The replication level of a virus can be determined, in other embodiments, using techniques known in the art, and in other embodiments, as exemplified herein. For example, the genome level can be determined using RT-PCR, and northern blot. To determine the level of a viral protein, one can use techniques including ELISA, immunoprecipitation, immunofluorescence, EIA, RIA, and Western blotting analysis.

In one embodiment, the invention provides a method of identifying sequences in HCV associated with HCV pathogenicity, comprising contacting cells with an isolated nucleic acid molecule encoding an infectious recombinant HCV genome, contacting cells with an isolated nucleic acid molecule comprising at least one mutation, independently culturing the cells and determining HCV infection, replication, or cell-to-cell spread, in cells contacted with the mutant, versus the recombinant HCV, whereby changes in HCV infection, replication, or cell-to-cell spread in cells contacted with the mutant virus shows the mutation is in an HCV sequence associated with HCV pathogenicity.

In one embodiment, the invention provides a method of identifying HCV variants with improved growth in cell culture, the method comprising contacting cells with an isolated nucleic acid molecule encoding an infectious recombinant HCV genome contacting cells with an isolated nucleic acid molecule comprising at least one mutation, independently culturing the cells and determining HCV infection, replication, or cell-to-cell spread, in cells contacted with the recombinant HCV or the mutated virus, whereby enhanced HCV infection, replication, or cell-to-cell spread in cells contacted with the mutated virus shows that the HCV variant has improved growth in cell culture.

In some embodiments, HCV variants are selected for enhanced replication, over a long course of time, in vitro culture systems. According to this aspect of the invention, and in some embodiments, cells contacted with the variants are characterized by reduced infection, as compared to cells contacted with the recombinant HCV.

### Kits

In a related aspect, the invention also provides a test kit for HCV comprising HCV virus components, and a diagnostic test kit for HCV comprising components derived from an HCV virus as described herein.

Furthermore, the invention also provides test kits, for screening for new HCV inhibitors, neutralizing and cross neutralizing antibodies, comprising HCV virus components.

A further aspect of the present invention relates to a method for obtaining an isolated nucleic acid molecule encoding a human hepatitis C virus with adaptive mutations, comprising identification of one or more adaptive mutations as described in the above method, incorporation of said one or more adaptive mutations into a nucleic acid molecule encoding a full length human hepatitis C virus or a fragment hereof, and isolating the nucleic acid molecule encoding a human hepatitis C virus with adaptive mutations.

One embodiment of the present invention relates to an isolated nucleic acid molecule obtained from the above method.

### General

Reference to any prior art in this specification is not, and should not be taken as, an acknowledgment or any form of suggestion that this prior art forms part of the common general knowledge in any country.

All patent and non-patent references cited in the present application, are hereby incorporated by reference in their entirety.

As will be apparent, preferred features and characteristics of one aspect of the invention may be applicable to other aspects of the invention. The invention may be embodied in other specific forms without departing from the spirit or essential characteristics thereof. The foregoing embodiments are therefore to be considered in all respects illustrative rather than limiting on the invention described herein. Scope of the invention is thus shown by the appended claims rather than by the foregoing description, and all changes that come within the meaning and range of equivalency of the claims are intended to be embraced by reference therein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In addition, singular reference does not exclude a plurality. Thus, references to “a”, “an”, “first”, “second” etc. do not preclude a plurality.

It should be noted that embodiments and features described in the context of one of the aspects of the present invention also apply to the other aspects of the invention.

The invention will hereinafter be described by way of the following non-limiting Figures and Examples.

### EXAMPLES

### Example 1-Materials & Methods

### HCV Recombinants

Original TNcc, J6cc and DBN3acc recombinants were developed previously (Li et al., 2012a; Li et al., 2012b; Ramirez et al., 2016). High-yield HI-recombinants were engineered using subclones of cell culture derived reverse transcription polymerase chain reaction (RT-PCR) amplicons and In-Fusion technology. Recombinants with genotype (isolate) 1a (TN), 1b (J4), 2a (J6), 2b (J8), 3a (S52), 3a (DBN), 4a (ED43), 5a (SA13) and 6a (HK6a) (Lindenbach et al., 2005; Gottwein et al., 2009; Scheel et al., 2011) specific Core-NS2 and remaining sequences of genotype 2a isolate JFH1 were used in in vitro HCV neutralization assays.

### Plasmids Encoding Cell Culture Infectious HCV Recombinants

Original cell culture infectious genotype 1a strain TN (TNcc), genotype 2a strain J6 (J6cc) and genotype 3a strain DBN (DBN3acc) recombinants were developed previously (Li et al., 2012 (a); Li et al., 2012 (b); Ramirez et al., 2016). High-yield HI-recombinants were engineered based on generated subclones harboring the desired mutations using In-Fusion technology. In brief, 50 ng of gel purified PCR amplicons generated using the subclones with the sequences of interest as templates were incubated with 2 μl of 5× In-Fusion HD enzyme (Takara Bio) and H₂O added to a total volume of 10 μl. The reaction was incubated for 15 min at 50° C. and 4 μl of the mix was transformed in 50 μl Stellar Competent Cells (Takara) according to the manufacturers' protocol.

Previously developed cell culture infectious HCV recombinants with genotype (isolate) 1a (TN) [GenBank accession number: HQ852453], 1b (J4) [GenBank accession number: FJ230881], 2a (J6) (described in Lindenbach et al. 2005), 2b (J8) [GenBank accession number: FJ230882], 3a (S52) [GenBank accession number: EU204645], 3a (DBN) [GenBank accession number: HQ852457], 4a (ED43) [GenBank accession number: EU363760], 5a (SA13) [GenBank accession number: FJ393024], 6a (HK6a) [GenBank accession number: FJ230883] (Lindenbach et al, 2005; Gottwein et al., 2009; Scheel et al., 2011) specific Core-NS2 and remaining sequences of genotype 2a isolate JFH1 were used in in vitro neutralization assays. Except for 6a (HK6a) containing two vital cell culture adaptive substitutions in the envelope proteins, these viruses reflected the consensus envelope protein sequences determined in vivo. For all plasmid preparations used in experiments the complete HCV sequence was verified by Sanger sequencing (Macrogen).

### Cells

Human hepatoma Huh7.5 cells were used for propagation of HCV, and human embryonic kidney HEK293 cells were used for production of HCV E1/E2 complexes.

### Maintenance of Huh7.5 and HEK293 Cells

The human hepatoma cell line Huh7.5 (Gottwein et al., 2009) and the human embryonic kidney cell line HEK293 were cultured with Dulbecco's modified Eagle medium (DMEM) (Invitrogen) containing 10% fetal bovine serum (Sigma), penicillin 100 U/ml and streptomycin 100 μg/ml (P/S) (Gibco/Invitrogen) at 37° C. and 5% CO₂; cultures were split every 2-3 days using trypsin (ThermoFisher Scientific) as described (Gottwein et al., 2009).

### Transfection of HCV In Vitro RNA Transcripts in Huh7.5 Cells

In vitro HCV RNA transcripts were produced using T7 RNA polymerase (Promega) and transfections were carried out using Lipofectamine2000 (Invitrogen) as described (Gottwein et al., 2009).

Briefly, plasmids were linearized with XbaI (New England Biolabs) following the sequence encoding the 3′end of the HCV genome, and RNA in vitro transcription reactions were carried out using T7 RNA polymerase (Promega). RNA concentrations were measured using the Qubit RNA BR Assay Kit (Thermo Fisher Scientific); for recombinants directly compared in the same graph, equal amounts of RNA transcripts were used. Prior to the day of transfection, 400,000 cells were seeded per well of a 6 well plate (Nunc); 7.6 μg of TNcc or TNcc-HI, 7.5 μg of J6cc or J6cc-HI, and 5 μg of DBN3acc or DBNcc-HI RNA transcripts were combined with 5 μl Lipofectamine2000 (Invitrogen) in a total of 500 μl Opti-MEM (Gibco/Invitrogen) and cells were incubated with the resulting transfection complexes in a total of 2 ml Opti-MEM for 4-6 h prior to washing with PBS and addition of serum containing medium (DMEM+10% FBS+P/S). Transfected cultures were split every 2-3 days. When cells were split, replicate cell cultures were plated in chamber slides for immunostaining to monitor the % of infected cells. In addition, supernatant was collected and stored at −80° C. for determination of HCV infectivity titers.

Infection of Huh7.5 Cells with HCV

Cells were inoculated with supernatants derived from transfection experiments at the peak of infection as described (Gottwein et al., 2009).

In brief, 400,000 cells/well in 6 well plates plated the previous day, were inoculated at the specified multiplicity of infection (MOI) using supernatants derived from the transfection experiment at the peak of infection, as determined by immunostaining and infectivity titration. Infected cultures were split every 2-3 days. When cells were split, replicate cell cultures were plated in chamber slides for immunostaining to monitor the % of infected cells. In addition, supernatant was collected and stored at −80° C. for determination of HCV infectivity titers.

### Serial Passage of HCV in Huh7.5 Cells

Cells were inoculated with culture supernatant derived from the previous passage at the peak of infection as described (Mathiesen et al., 2015).

In brief, for serial passage of cell culture infectious HCV recombinants, 10⁶ cells were seeded into T25 flasks (ThermoFisher Scientific) and the following day inoculated with 1 ml cell culture supernatant derived from the previous passage at the peak of infection as determined by immunostaining. The first passage culture was inoculated with 1 ml of supernatant derived from a prior transfection culture at the peak of infection. When cells were split, replicate cell cultures were plated in chamber slides for immunostaining of HCV antigen to monitor the % of infected cells and supernatant was collected and stored at −80° C. for determination of HCV infectivity titers.

### Generation of Virus Stocks in Huh7.5 Cells

Cells were inoculated with culture supernatants derived at the peak of infection. Supernatants collected around the peak of infection were pooled. For virus stocks used in neuralization assays, the HCV envelope protein sequence was confirmed by Sanger sequencing. For virus seed stocks for vaccine virus production, the complete HCV open reading frame (ORF) was analyzed by next generation sequencing (NGS).

In brief, 6×10⁶ cells were plated in T175 flasks for generation of HCV stocks for neutralization assays and of seed stocks for vaccine virus production, and the next day inoculated with cell culture supernatant derived from the peak of infection. When cells were split, replicate cell cultures were plated in chamber slides for immunostaining to monitor the % of infected cells. In addition, supernatant was collected and stored at −80° C. for determination of HCV infectivity titers. Supernatants derived on days where a high % of HCV infected cells was recorded by immunostaining, and/or a high infectivity titer was recorded, were pooled and used as virus stocks. For virus stocks used in neuralization assays, the envelope protein sequence was confirmed by Sanger sequencing. For virus seed stocks for vaccine virus production, the complete HCV open reading frame (ORF) was analyzed by next generation sequencing (NGS).

### Immunostaining of HCV Antigens in Cell Culture

The % of HCV infected cells was monitored by immunostaining using primary antibodies monoclonal anti-HCV core antibody C7-50 (EnzoLifeSciences) diluted 1:5000 and monoclonal anti-HCV NS5A antibody 9E10 (Lindenbach et al., 2005) diluted 1:5000 as well as secondary antibody Alexa Fluor 488 goat anti mouse IgG (H+L) (Invitrogen) diluted at 1:500 as described (Gottwein et al., 2009).

In brief, immunostaining of HCV antigens was done on replicate cultures derived when HCV infected cells were split using primary monoclonal anti-HCV core antibody C7-50 (EnzoLifeSciences diluted 1:5000 in BSK (PBS supplemented with 0.2% skim milk (Easis) and 1% Bovine Serum Albumin, (Sigma)) and monoclonal anti-HCV NS5A antibody 9E10 (Lindenbach et al., 2005) diluted 1:5000 in BSK (Gottwein et al., 2009). After at least one hour of incubation and subsequent washing, secondary antibody Alexa Fluor 488 goat anti mouse IgG (H+L) (Invitrogen) diluted 1:500 in BSK was added and cell nuclei were counterstained with Hoechst reagent (Invitrogen) (Gottwein et al., 2009; Mathiesen et al. 2014). The % of infected cells was scored from 0% to 100%, using intervals of 10%, by fluorescence microscopy with a Zeiss Axio Vert.A1 microscope.

### Determination of HCV Infectivity Titers

HCV infectivity titers in cell culture supernatants were determined as focus-forming units (FFU)/ml by titration on 96-well plates and subsequent immunostaining using primary antibodies anti-HCV core antibody C7-50 (EnzoLifeSciences, Farmingdale, NY) diluted 1:1000 and anti-HCV NS5A antibody 9E10 diluted 1:3000 as well as secondary antibody ECL sheep anti-mouse IgG diluted 1:500, followed by visualization and automated counting of FFU as described (Pham et al., 2019). Thus, HCV infectivity titers were determined in cell culture supernatant as focus-forming units (FFU)/ml as previously described (Gottwein et al., 2009).

In brief, 6000 cells were seeded per well of 96 well plates (Nunc) and infected the following day with serially diluted cell culture supernatants, testing each dilution in triplicate. After 48 h of incubation, cells were fixed using methanol and stained for HCV antigens using primary anti-HCV core antibody C7-50 (EnzoLifeSciences) diluted 1:1000 in BSK and anti-HCV NS5A antibody 9E10 diluted 1:3000 in BSK, followed by secondary antibody ECL sheep anti-mouse IgG diluted 1:500 in BSK (Mathiesen et al., 2014). The HCV antigen positive cells were visualized and FFU were automatically counted using an Immunospot series 5 UV analyzer (CTL Europe GmbH) as described (Gottwein et al., 2009).

### Sequencing of Cell Culture Derived HCV

HCV RNA was extracted from culture supernatants and either the complete ORF (for serial passage experiments, first passage kinetic experiments and virus seed stocks) or E1/E2 (for virus stocks for neutralization assays) were amplified by RT-PCR using specific primers (Table 1-2) followed by NGS for ORF amplicons or Sanger sequencing for E1/E2 amplicons as described (Pham et al., 2019; Jensen et al. 2019).

In brief, HCV RNA was extracted from cell culture supernatants using Trizol LS (Life technology) and the RNeasy Minelute kit (QIAGEN) as described (Jensen et al., 2019). RNA was eluted and used for reverse transcription with Maxima H Minus Reverse Transcriptase (ThermoScientific) and genotype specific reverse primers binding to the HCV 3′UTR variable region as described (Jensen et al., 2019; Pham et al., 2019) (Table 1). Polymerase chain reaction (PCR)-based amplification of the complete open reading frame (ORF) was carried out using Q5 Hot start High-Fidelity DNA polymerase and genotype specific primers as described (Jensen et al., 2019; Pham et al., 2019) (Table 2). Alternatively, for virus stocks used for neutralization assays, a PCR amplicon spanning the envelope proteins was generated (Table 2).

The sequence of the resulting PCR amplicons was either determined by Sanger sequencing or by NGS. NGS was carried out as described (Jensen et al., 2019). In brief, PCR amplicons were loaded on a gel with SYBR safe DNA gel stain (Invitrogen), visualized with blue light and gel extracted with the Large Fragment DNA recovery kit (Zymo Research). Size selection was done with magnetic beads and adaptors with unique barcodes with the use of TruSeq Nano DNA library kit (Illumina). Finally, pair-end sequencing was performed on the Miseq platform. Viruses from serial passage experiments, first passage kinetic experiments and virus seed stocks used for vaccine production, were subjected to NGS of the complete ORF. Virus stocks for neutralization assays were subjected to Sanger sequencing of genome regions encoding E1 and E2.

### Subclonal Analysis

Selected RT-PCR ORF amplicons were subcloned using the TOPO-XL Cloning kit (Invitrogen) followed by Sanger sequencing.

In brief, PCR amplicons spanning the complete ORF were subcloned for serially passaged viruses using the TOPO-XL Cloning kit (Invitrogen) following the manufacturers' instructions. The HCV sequence of the resulting plasmids was determined by Sanger sequencing. TNcc-PP-10, TNcc-PP-18, TNcc-PP-38.1, J6cc-PP-35 and DBNcc-PP-16 were analyzed like this. For TNcc-PP-10, TNcc-PP-18 and TNcc-PP-38.1, phylogenetic analysis was carried out using MAFFT for aligning sequences and subsequently PhyML for building the phylogeny visualized by FigTree v1.4.3.

### HCV Production for Vaccine Generation

HCV was produced in serum-free medium in 10-layer cell factories as described (Mathiesen et al., 2014; Pihl et al., 2022).

HCV production for immunization studies was done using previously developed protocols (Phil et al., 2022). In brief, 18×10⁶ cells were plated in T500 triple layer cell culture flasks (ThermoFisher Scientific). The next day, cells were infected at MOI 0.003 with genotype 1a, 2a and 3a seed stocks. On day 1 post infection, around 1.8×10⁸ cells were plated in 10-layer cell factories (ThermoFisher Scientific). When 80% of cells were estimated to be infected by monitoring of a replicate T25 cell culture, cells in the cell-factory were washed with pre-warmed PBS and DMEM was replaced by Adenovirus Expression Medium (AEM) supplemented with P/S (Mathiesen et al., 2014). From the 10-layer cell factories, supernatant was harvested every 2-3 days. Each factory yielded 5 harvests of 800 ml each. For each HCV recombinant, 4 cell factories were done, collecting a total volume of ~16L of virus containing supernatant. Harvested supernatant was stored at −80° C. until further processing.

### Processing of HCV for Vaccine Generation

HCV was clarified using 5 μm and 0.65 μm filters and further concentrated by tangential flow filtration (TFF) with a molecular weight cut off (MWCO) of 500 kDa, (Lothert et al., 2020) followed by two ultracentrifugation steps using Optiprep Density Gradient Medium (Sigma) for formation of 3 density cushions and a continuous gradient, respectively, separated by an intermediate TFF step (MWCO 500 kDa). Following a final size exclusion chromatography using Sephadex G-100 (Sigma Aldrich), HCV was UV-irradiated with a UVG-54 Handheld UV lamp (240 nm UV, 6 watt) (Analytik Jena) (Pihl et al., 2022).

In brief, initial clarification of ~16L virus containing supernatant harvested from cell factories was carried out through a 5 μm capsule filter, followed by a 0.65 μm Sartopure® PP3 filter (Sartorius). Afterwards, two tangential flow filtration (TFF) steps were carried out using a fiber with a surface area (SA) of 2600 cm² and a molecular weight cut off (MWCO) of 500 kDa and subsequently a fiber with a SA of 790 cm² and a MWCO of 500 kDa (MINIKROS 65 cm 500KD MPES, MINIKROS 20 cm 500KD MPES, Repligen), concentrating ~16L to ~350 ml and subsequently to ~35 ml. For a subsequent 3-cushion ultracentrifugation step, 3 cushions containing 1 ml of 60%, 28% and 10% Optiprep Density Gradient Medium (Sigma), respectively, were layered on top of each other in 6 ultracentrifugation tubes. To each tube, ~6 ml of virus sample were added and the total volume was adjusted to 11 ml with PBS. After centrifugation with a Beckman SW-41 rotor at 40,000 revolutions per minute (rpm) at 4° C. for 2h, fraction 1 (8 ml), fraction 2 (1.2 ml) and fraction 3 (2 ml) were collected from the top of each tube.

Fraction 2 was pooled from 6 tubes and adjusted to a total volume of 20 ml with PBS prior to TFF with a fiber with a SA of 20 cm² and a MWCO of 500 kDa (MICROKROS 20 CM 500K MPES 0.5 MM, Repligen) resulting in concentration to ~2 ml. For a subsequent equilibrium density gradient ultracentrifugation step one day prior to the experiment a semi-continuous 40% to 10% iodixanol gradient was prepared by layering 2.5 ml of 40%, 30%, 20% and 10% Optiprep Density Gradient Medium on top of each other followed by equilibration at 4° C. The ~2 ml sample resulting from the previous step was added on top of the gradient and ultracentrifugated at 40,000 rpm with a Beckman SW-41 rotor for 6 h at 4° C. Eighteen fractions of ~550 μl were collected from the bottom of the tube and each fraction was weighed for determination of buoyant density. The three fractions with a density closest to 1.1 g/ml were pooled, obtaining a final volume of ~1.5 ml. For a subsequent size exclusion chromatography step 1.54 g of Sephadex G-100 (Sigma Aldrich) was mixed with 35 ml sterile water 3 days prior to the experiment and added to a chromatography PD-10 column (PD-10 reservoirs, GE Healthcare Life Science). The ~1.5 ml of sample resulting from the previous step were then added to the column, and 12 fractions of ~1 ml were eluted with NaCl (9 mg/ml). Based on absorbance determined with a NanoDrop (Thermo Scientific) at an OD of 230 nm, 5 fractions were pooled, obtaining a final volume of close to 5 ml. Finally, the resulting preparation was subjected to UV-irradiation with a UVG-54 Handheld UV lamp (240 nm UV, 6 watt) (Analytik Jena) for 25 min with frequent agitation using a 6 well plate with 1.25 ml of sample per well. To confirm inactivation, cells seeded one day prior to the experiment were inoculated with 100 μl of UV inactivated sample, and cell cultures were followed for 3 weeks by splitting and immunostaining every 2-3 days.

### Immunization of Mice

6-8 week-old BALB/c mice (Taconic Farms, Denmark) were subcutaneously immunized four times every 3 weeks with HCV or OVA formulated with adjuvant AddaVax 50%/50% (v/v).

In brief, 6-8 week-old female BALB/c mice were acquired from Taconic Farms, Denmark, and were housed in certified animal facilities at the University of Copenhagen, to evaluate immunogenicity of the developed vaccine candidates. Animals were resting for at least one week following arrival to the animal facility. Animals were subcutaneously immunized four times every 3 weeks with processed and inactivated HCV or as a control with 100 μg EndoFit OVA (Invitrogen) formulated with adjuvant AddaVax 50%/50% (v/v). Genotype 1a, 2a and 3a HCV vaccines contained an equivalent of 6.8, 8.6, and 8.6 log 10 FFU, respectively, determined prior to inactivation. Each experimental group including the control group consisted of 3 animals. Thus, a total of 12 animals were used. Animals were randomly assigned to the different groups and treatments were administered in random order. The size of the groups was determined based on availability of vaccine antigen. Two weeks after the last immunization mice were sacrificed and serum was obtained. IgG was purified from serum of each animal and was evaluated in in vitro neutralization assays as the primary outcome measure; in addition IgG pools derived from each group were evaluated in in vitro neutralization assays as the primary outcome measure and in HCV E1/E2 complex ELISA assays as a secondary outcome measure. No animals were excluded from the study. All data obtained from all animals were reported. During the experiments, persons handling animals and samples were blinded to the identity of the vaccine antigen.

### Patient Samples

Sera or plasma from patients with chronic hepatitis C (CHC) were collected between May 2011 and August 2021 in biobanks attached to the Danish Database for Hepatitis B and C and the HCV Tandem cohort at the Department of Infectious Diseases, Copenhagen University Hospital, Hvidovre. Patients were ≥18 years, had no previous history of treatment for CHC, no co-infection with human immunodeficiency virus (HIV) or hepatitis B virus, and no recent intravenous drug use.

### Purification, Concentration and Quantification of IgG

IgG was purified from mouse serum or patient serum or plasma with the Amicon® Pro Affinity Concentration Kit Protein G with 50 kDa Amicon® Ultra-0.5 Device (Merck Millipore), concentrated with the Vivaspin 500, 30,000 MWCO (GE Lifescience) kit. Mouse and patient IgG was quantified with the IgG (TOTAL) mouse uncoated ELISA Kit (ThermoFisher) and the Cedex Bio Analyzer (Roche), respectively.

IgG from individual mouse serum or individual patient serum/plasma samples was purified with the Amicon® Pro Affinity Concentration Kit Protein G with 50 kDa Amicon® Ultra-0.5 Device (Merck Millipore), in accordance to the manufacturers' instructions. Briefly, for mouse samples 200 μl and for patient samples 600 μl of Protein G resin was added to the column, followed by a wash step. Next, serum or plasma was added and incubated for 1 h at room temperature on a shaker. Following the second wash, elution and neutralization buffer were added to obtain IgG.

Concentration of IgG was done with the Vivaspin 500, 30,000 MWCO (GE Lifescience) kit according to the manufacturers' instructions. Briefly, IgG resulting from the previous step was diluted in PBS to a total volume of 500 μl and centrifugated at 14,000 g until a volume of ~80 μl was obtained.

Concentrations of the resulting mouse IgG preparations were determined with the IgG (TOTAL) mouse uncoated ELISA Kit (ThermoFisher) according to the manufacturers' instructions. Briefly, 96 well plates were coated with capture antibody and incubated overnight, followed by incubation with blocking buffer. After several washes, concentrated and serially diluted IgG was added in duplicates together with the provided standards. Then, the detection antibody was added and incubated for two hours on a shaker. Next, substrate was added followed by stop solution and absorbance at an OD of 490 nm was measured (FLUOstar OPTIMA, BMG Labtech). The generated standard curve was used to calculate the IgG concentration.

Concentrations of the resulting patient IgG preparations were determined with the Cedex Bio Analyzer (Roche) according to the manufacturers' instructions.

### In Vitro Neutralization Assay

HCV neutralization with mAb AR3A and AR4A (Law et al., 2008; Giang et al., 2012) and polyclonal antibody C211 (Prentoe et al., 2019) was done in a volume of 100 μl followed by inoculation of Huh7.5 cells plated on 96-well plates, which were subsequently subjected to immunostaining of HCV antigen and automated counting of FFU (Mathiesen et al., 2015; Mathiesen et al., 2014). HCV neutralization with purified mouse or patient IgG was done similarly in a volume of 10 μl (Czarnota et al., 2020). % neutralization was calculated as 100-[100×(FFU count in experimental wells)/(mean FFU count in virus only wells)].

Neutralization with human derived monoclonal antibodies AR3A and AR4A, (Law et al., 2008; Giang et al., 2012) and polyclonal IgG C211 (Prentoe et al., 2019) was done as described in (Gottwein et al., 2009). In brief, these antibodies were serially diluted in cell growth medium (DMEM+10% FBS+P/S), added to the virus and incubated in a total of 100 μL for 1 h at 37° C. Each antibody concentration was tested in triplicate. Virus only controls were prepared by mixing virus with cell growth medium. Virus-antibody mixes, virus-medium mixes or medium only were then added to Huh7.5 cells, which had been seeded at 6000 cells/well in 96 well poly-D-Lysine coated plates (Nunc) the day prior to the experiment. Following 3.5 h incubation, cells were washed with PBS and 100 μL cell growth medium was added per well. After 48 h of incubation at 37° C., plates were fixed with methanol and stained for HCV NS5A antigen as described for infectivity titrations.

Neutralization with purified mouse serum IgG or patient serum/plasma IgG was done using previously established assays (Czarnota et al., 2020). In brief, these IgG were serially diluted in cell growth medium in a total of 3 μl and added to virus diluted in 7 μl to a total of 10 μl and incubated for 1.5 h at 37° C. Each antibody concentration was tested in triplicate. Virus only controls were prepared by mixing virus with cell growth medium. Subsequently, 30 μl of cell growth medium were added to virus-IgG or virus-medium mixes and the resulting samples were added to Huh7.5 cells, which had been seeded at 6000 cells/well in 96 well poly-D-Lysine coated plates (Nunc) the day prior to the experiment. Following 4.5 h incubation at 37° C., cells were washed with PBS and 100 μl of cell growth medium were added per well. As a positive neutralization control, C211 antibody and a well-defined genotype 5a virus (Mathiesen et al., 2015) were mixed and included in each experiment. After 48 h of incubation at 37° C., plates were fixed with methanol and BSK was added at 300 μl/well. Following 1 h incubation, BSK was removed and 100 μg/mL Fab Fragment Goat anti-mouse IgG (Jackson ImmunoResearch) diluted in PBS was added and incubated for 1 hour at room temperature. Next, plates were washed and 50 μl of monoclonal primary antibody 9E10 diluted 1:5000 in BSK was added per well. The remaining staining steps were done as described above for infectivity titrations.

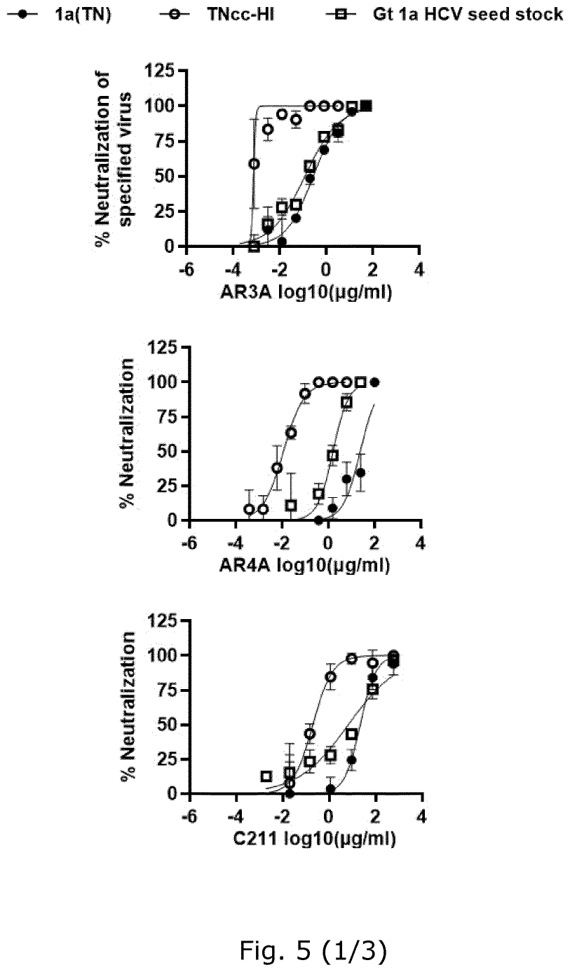

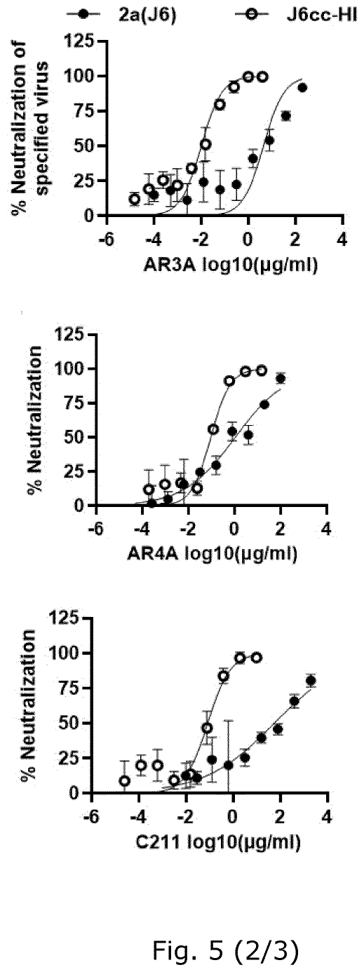

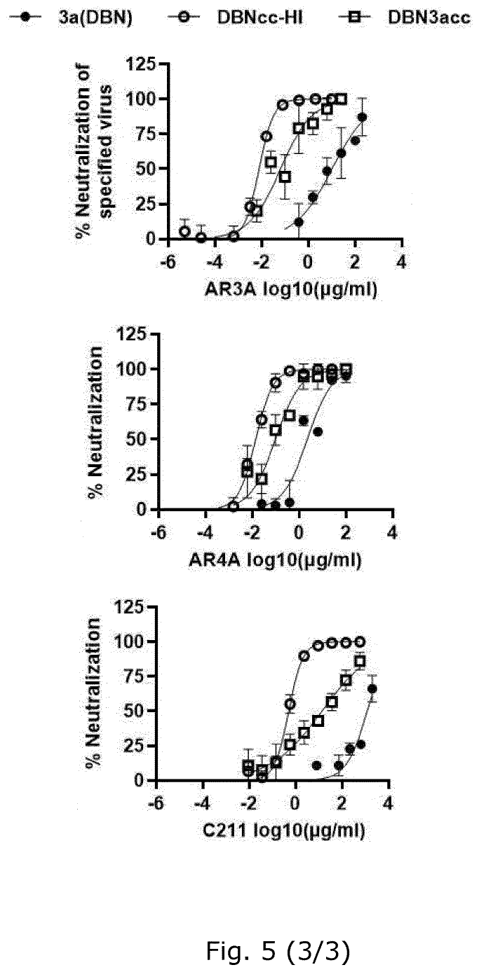

For all neutralization assays, the percentage of neutralization was calculated relating the number of FFU in experimental wells to the mean number of FFU in virus only wells. For all neutralization assays, EC50 were calculated based on concentration-response curves, using top and bottom constrains of 0 and 100% and the formula y=100/(1+10^{(log 10EC50-X)×hillslope}) using GraphPad Prism, as described (Mathiesen et al., 2014). Fold-changes in neutralization sensitivity given in Tables 7, 8 and 9, based on data in FIG. 5, were calculated as [(EC50 of original virus)/(EC50 of virus with envelope substitutions)]; these calculated values are not meant to reflect data accuracy.

### HCV E1/E2 Complex ELISA

E1/E2 complexes were obtained from lysates of HEK293 cells transfected with E1/E2 expression plasmids. Binding of mouse IgG or immune-sera to E1/E2 complexes was evaluated by ELISA using secondary antibody ECL sheep anti-mouse IgG horseradish-peroxidase linked whole antibody (GE Healthcare) diluted 1:1000. As positive control, mAb AP33 (Clayton et al., 2002) and H77.39 (Sabo et al., 2011) were used. As negative control, secondary antibody only was used.

Expression plasmids harboring E1 and E2 envelope protein sequences of TNcc-HI, J6cc-HI and DBNcc-HI were constructed using the In-Fusion HD cloning kit (Takara Bio) and 50 ng of PCR amplicons harboring either E1 and E2 sequences of the TNcc-HI, J6cc-HI or DBNcc-HI plasmids and an HCV pseudo-particle expression plasmid. The E1 and E2 sequence in the resulting plasmids was sequence confirmed (Macrogen). Plasmids were transfected in HEK293 cells, plated at 8×10⁴ cells/well in 6-well plates 1 day prior to the experiment, using 5 μl Lipofectamine2000 (Invitrogen) and 5 μg plasmid in a total volume of 2 ml of Opti-MEM (Invitrogen). Following 6 h of incubation, Opti-MEM was replaced with DMEM+10% FBS+P/S. In a replicate culture, after 48 h of incubation at 37° C., transfection efficiency was determined by immunostaining for E1/E2 protein using the monoclonal antibody AR4A (Law et al., 2008) diluted 1:5000 in BSK for one hour at room temperature, followed by application of the anti-human Alexa Fluor 488 coupled secondary antibody, as described above. Following confirmation of expression, cell growth medium was discarded, and total protein was collected in lysate buffer (1% Triton X-100, 50 nM Tris-HCl, 150 nM NaCl, pH8.0). Next, the cell lysate was treated with Benzonase endonuclease (Sigma) and 2 nM of MgCl₂ and finally, the generated protein was measured with BCA protein assay kit (Pierce™) according to the manufacturers' instructions.

For ELISA assays, 96-well plates (Nunc) were coated with Galanthus nivalis lectin (Medicago) and the next day the plate was washed with PBS and blocked with 200 μl of PBS-5% non-fat milk (Åblomst). The next day, the plates were washed with PBS containing 0.1% Tween (PBST) and 50 μl of E1/E2 complexes (200 μg/ml) were added to the plates and incubated at 4° C. overnight. After several washes with PBST, purified mouse serum IgG or immune-sera were serially diluted in PBST-1% non-fat milk, dilutions were added to plates in duplicates, and plates were incubated for 2 h at room temperature, followed by a washing step with PBST. Binding of antibodies to E1/E2 complexes was detected by secondary antibody ECL sheep anti-mouse IgG horseradish-peroxidase linked whole antibody (GE Healthcare) diluted 1:1000 in PBST-1% non-fat milk and incubated for 1 h at room temperature, followed by a washing step with PBST. TMB substrate (3,3′, 5,5′-tetramethylbenzidine, Thermo Scientific) was then added for 10 min followed by ELISA Stop Solution (Invitrogen).

Absorbance was determined at 450 nm with the use of an ELISA plate reader (BIO-TEK Instruments, Inc.). As positive controls, instead of mouse serum IgG, mouse anti-E2 antibody AP33 (Sabo et al., 2011) was used to bind TNcc-HI E1/E2 and DBNcc-HI E1/E2 complexes and H77.39 (Prentoe et al., 2016) was used to bind J6cc-HI E1/E2 complexes. As negative control, only secondary antibody, ECL sheep anti-mouse IgG horseradish-peroxidase linked whole antibody (GE Healthcare) was added to the E1/E2 complexes omitting prior addition of mouse serum IgG, immune-sera or control antibodies.

### Example 2-Generation of High-Yield Genotype 1a, 2a and 3a Polyclonal HCV

### Aim

To develop high-yield culture systems.

### Materials and Methods

See example 1.

### Results

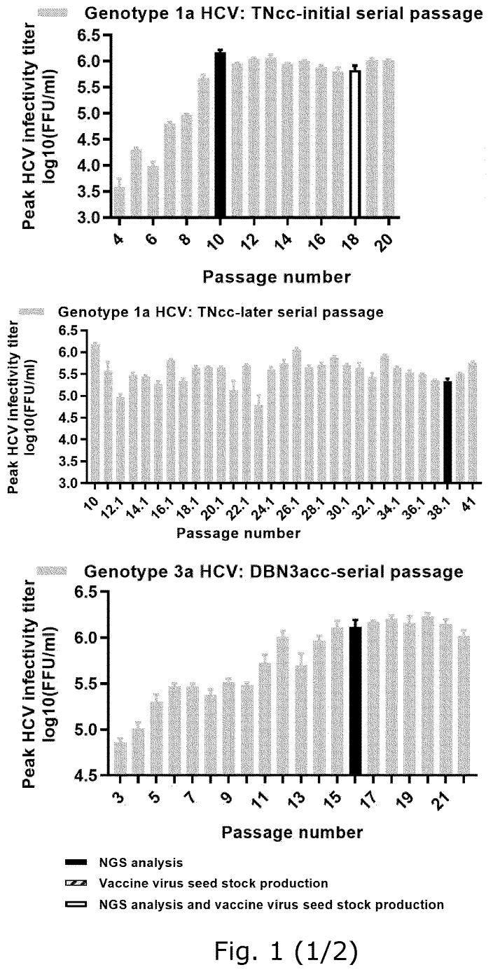

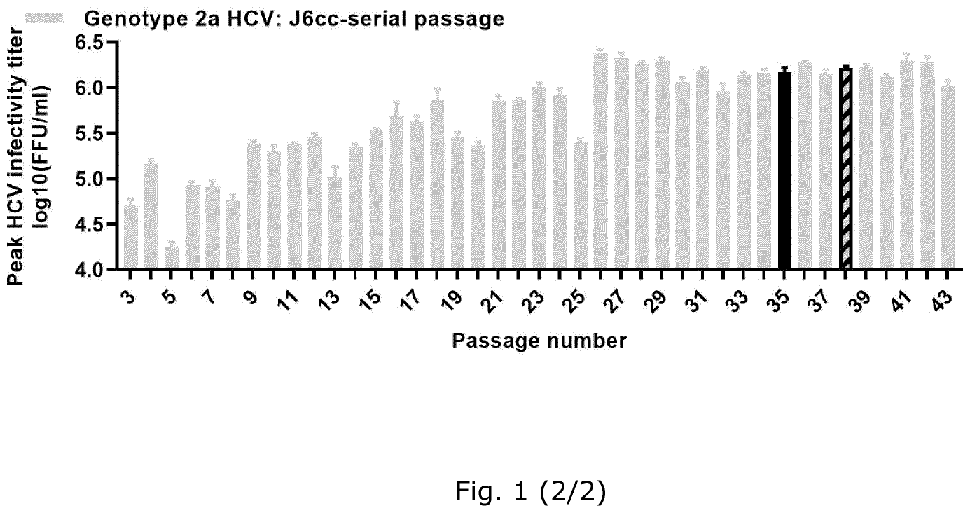

Full-length TNcc (genotype 1a) (Li et al., 2012 (a)), J6cc (genotype 2a) (Li et al., 2012 (b)) and DBN3acc (genotype 3a) (Ramirez et al., 2016) HCV recombinants were serially passaged in Huh7.5 hepatoma cells until peak HCV infectivity titers showed a plateau at ~6 log 10 FFU/ml (FIG. 1).

An additional criterion for termination of passaging was detection of putative cell culture adaptive substitutions in >80% of the viral population as determined by NGS (see example 3). For an initial TNcc passage line, NGS suggested viral quasispecies populations with mutations at a prevalence <80%, spurring a later passage line with a total of 41 viral passages. For J6cc and DBN3acc, 43 and 22 passages were done, respectively. Compared to the initial passages, late passages showed an increase in HCV infectivity titers of up to 2.6 log 10 for TNcc, 1.7 log 10 for J6cc and 1.3 log 10 for DBN3acc.

### Conclusion

Later passages for all three genotypes showed increased infectivity titers.

### Example 3-Genetic Changes Acquired by High-Yield Polyclonal Genotype 1a, 2a and 3a HCV

### Aim

To identify genetic correlates of high-yield phenotypes.

### Materials and Methods

See example 1.

### Results

NGS of the entire ORF of polyclonal passage (PP) viruses were carried out.

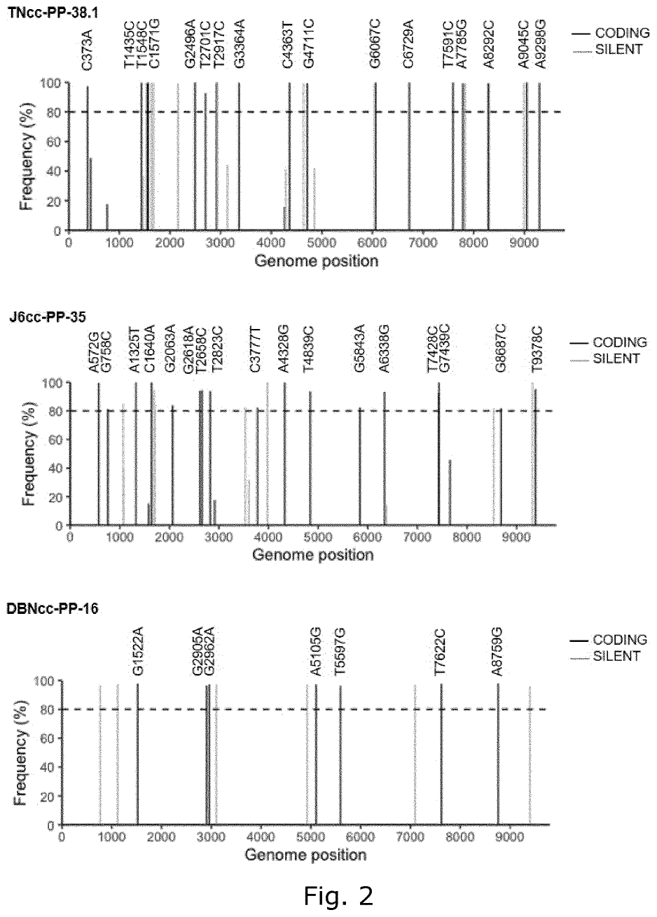

For TNcc-PP-10 and TNcc-PP-18, derived from passage 10 and 18 of the initial passage line, NGS suggested a viral quasispecies population with most coding nucleotide changes being present in <80% of viral genomes (Table 3). In contrast, for TNcc-PP-38.1 derived from the later passage line, as well as J6cc-PP-35 and DBNcc-PP-16, a more homogeneous viral population was found with most coding nucleotide changes present in ≥80% of viral genomes (FIG. 2 and Tables 4-6).

TNcc-PP-38.1, J6cc-PP-35, and DBNcc-PP-16 had 17, 17, and 7 coding changes being present in at least 80% of the viral population, among which 4, 3 and 1 localized to the envelope proteins, respectively. Of note, DBN3acc already harbored 5 coding changes in the envelope proteins compared to the consensus HCV sequence in the infected patient this recombinant was based on, while TNcc and J6cc did not contain coding changes in the envelope proteins (Li et al., 2012 (a); Li et al., 2012 (b); Ramirez et al., 2016). Subclonal analysis of these PP-viruses and phylogenetic analysis of TNcc-PP subclones reflected NGS results (Data not shown).

For the original TNcc, TNcc-PP-10, TNcc-PP-18 and TNcc-PP-38.1, generated subclones were subjected to phylogenetic analysis using PhyML; subclones for the original TNcc were generated and reported previously (Jensen et al., 2019). In comparison to the TNcc plasmid sequence, in passage 10 and 18 viruses, this analysis revealed two main quasispecies populations, designated A and B. The A population was mainly characterized by signature substitutions N2651H and H2986R. The B population was mainly characterized by signature substitutions T2357A, S2375G, M2834L and H2986R. In the passage 10 subclones, the A and B population had similar frequency, with 8/18 belonging to A and 7/18 to B, respectively. In the passage 18 subclones, the A population appeared to prevail over the B population with 15/21 and 3/21 subclones belonging to the A and B population, respectively. TNcc-PP-38.1 was characterized by population A signature substitutions and acquisition of additional substitutions corresponding to the substitutions found with >80% frequency in TNcc-PP-38.1 (Table 4). Thus, subclonal analysis described in this section reflected results obtained by NGS (Tables 3-4).

### Conclusion

TNcc-PP-38.1, J6cc-PP-35, and DBNcc-PP-16 had 17, 17, and 7 coding changes being present in at least 80% of the viral population, among which 4, 3 and 1 localized to the envelope proteins, respectively.

### Example 4-Engineering of high-yield genotype 1a, 2a and 3a HCV recombinants

### Aim

Based on genetic analysis of high-yield PP-viruses, high-yield (HI)-recombinants were engineered.

### Materials and Methods

See example 1.

### Result

TNcc-HI-18A and TNcc-HI-18B reflected the two main populations in the initial passage line (Table 3). TNcc-HI reflecting TNcc-PP-38.1 in the later passage line, J6cc-HI reflecting J6cc-PP-35, and DBNcc-HI reflecting DBNcc-PP-16 harbored coding nucleotide changes with >80% frequency in NGS in combinations confirmed by subclonal analysis; as an exception, TNcc-HI also harbored G32S found at 48% frequency (unless otherwise indicated, amino acid position numbers relate to the specified sequences (FIG. 2 and Tables 4-6).

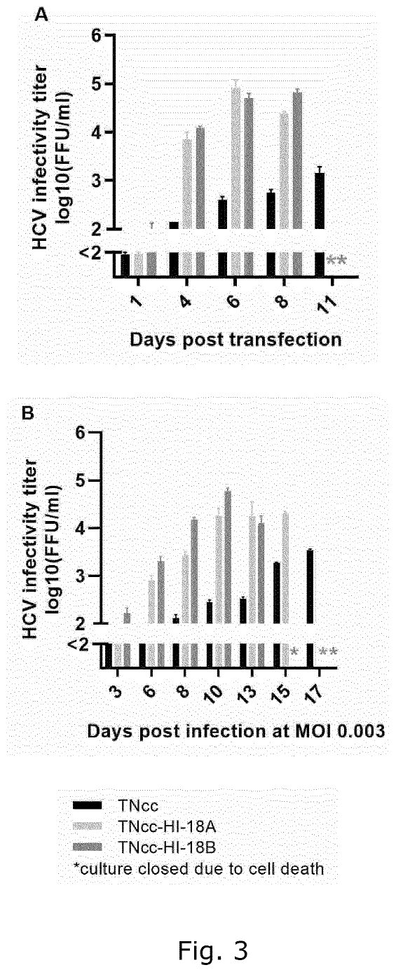

Compared to the original recombinants, all HI-recombinants showed increased fitness in transfection and first passage infection kinetic experiments, with accelerated spread kinetics, monitored by determination of the % of infected cells and of HCV infectivity titers, and with increased peak infectivity titers. TNcc-HI-18A and TNcc-HI-18B peak infectivity titers were only approaching 5 log 10 FFU/ml and thus fell short of the target of 6 log 10 FFU/ml (FIG. 3).

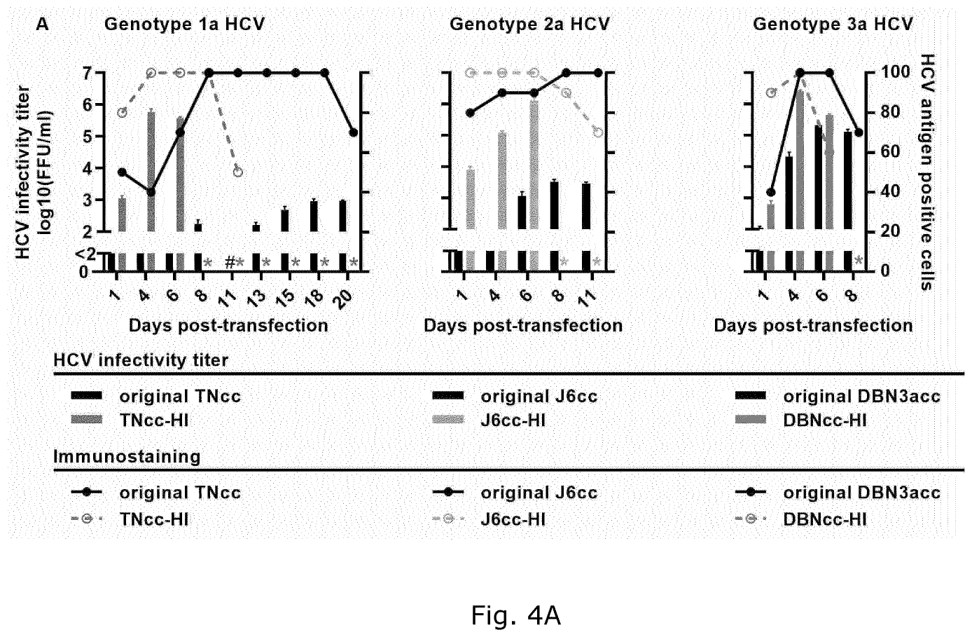

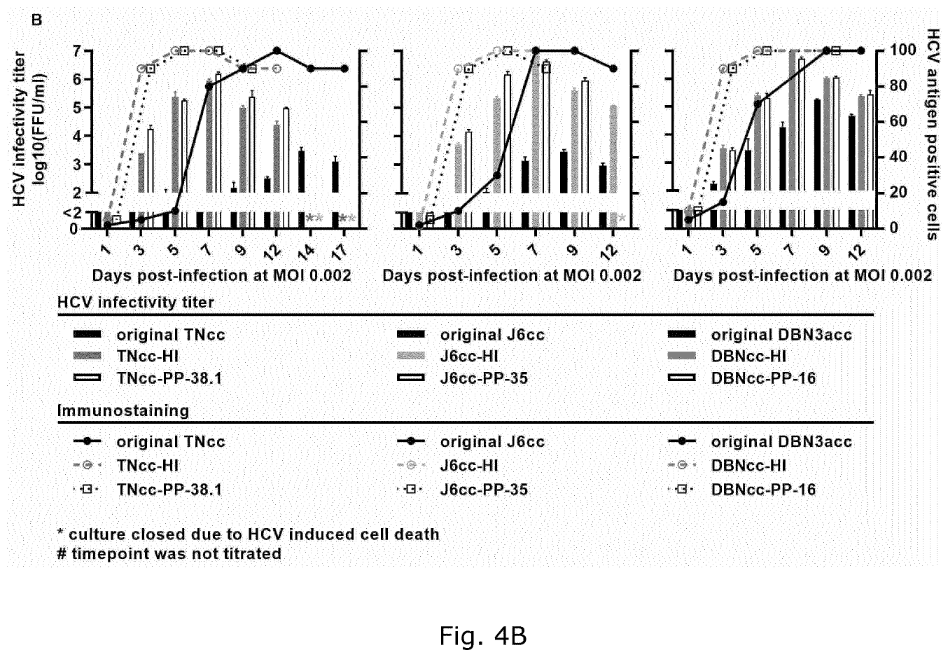

In contrast, in transfection/infection experiments, TNcc-HI, J6cc-HI and DBNcc-HI yielded peak infectivity titers of 5.8/6.0, 6.1/6.8, and 6.4/7.0 log 10 FFU/ml, respectively, while the respective original recombinants yielded 3.0/3.5, 3.5/3.4, and 5.3/5.3 log 10 FFU/ml, respectively (FIG. 4). In infection experiments, infectivity titers of HI-viruses were comparable to those of PP-viruses (FIG. 4B). HI-recombinants were genetically stable following first viral passage (no acquisition of substitutions with >10% frequency with exception of TNcc-HI, that acquired L179P and S1930Y with 15% and 39% frequency, respectively).

### Conclusion

HI-recombinants being genetically stable and having high infectivity titers were generated.

### Example 5-High-Yield Genotype 1a, 2a and 3a Recombinants Showed Increased Exposure of Neutralizing Epitopes

### Aim

To study exposure of neutralizing antibodies.

### Materials and Methods

See example 1.

### Results

Compared to the respective recombinants with in vivo derived envelope protein sequences without cell culture adaptive substitutions (Scheel et al., 2011; Lindenbach et al., 2005), based on determined EC50 values, HI-recombinants showed 12- to 2472-fold increased sensitivity to neutralization by human derived mAb AR3A (Law et al., 2008) and AR4A (Giang et al., 2012), targeting conserved conformational epitopes in E2 and E1/E2 associated with protection, respectively, and by polyclonal IgG C211 (Prentoe et al., 2019) derived from a patient chronically infected with genotype 1a (FIG. 5, Table 7, 8 and 9).

In detail, TNcc-HI showed 300-, 2400- and 110-fold increased neutralization sensitivity to AR3A, AR4A and C211, respectively, while a genotype 1a HCV seed stock derived from TN-PP-18 showed 3-, 15- and 3.2-fold increased sensitivity. J6cc-HI showed 440, 12- and 633-fold increased neutralization sensitivity. DBNcc-HI showed 1250-, 220- and 2472-fold, while DBN3acc showed 167-, 22- and 88-fold increased neutralization sensitivity.

For viruses with in vivo derived TN, J6 and DBN envelope protein sequences, determined half maximal effective concentrations (EC50) as shown in Tables 7-9, were in line with previously reported results (Prentoe et al., 2016; Carlsen et al., 2014; Augestad et al., 2020).

### Conclusion

Thus, HI-viruses showed greatly increased exposure of conserved conformational neutralizing epitopes associated with protection against chronic HCV infection.

### Example 6-Generation of Vaccine Candidates Based on Inactivated High-Yield Genotype 1a, 2a and 3a Recombinants

### Aim

To produce viruses for vaccine experiments HCV.

### Materials and Methods

See example 1.

### Results

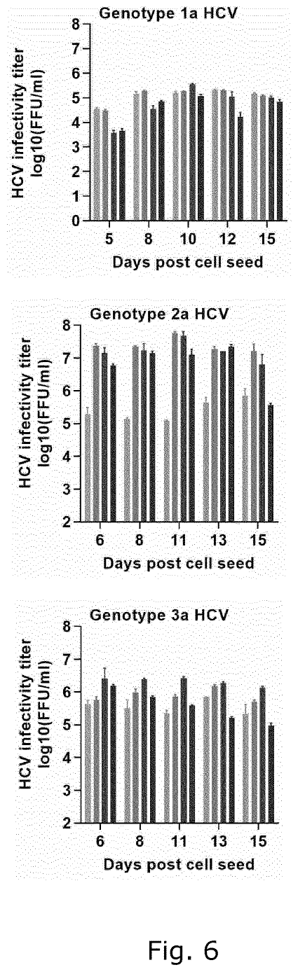

Seed stocks were generated by inoculation of Huh7.5 cells with polyclonal virus preparation TNcc-PP-18, polyclonal virus preparation J6cc-PP-35 or a first viral passage DBNcc-HI virus, available upon initiation of vaccine studies. Sequence confirmed genotype 1a, 2a and 3a HCV seed stocks with infectivity titers of 4.8, 6.2 and 6.4 log 10 FFU/ml, respectively (Tables 3, 5 and 6), were used to inoculate Huh7.5 cells for HCV production in 10-layer cell factories, resulting in a total volume of 16L HCV containing supernatant per virus (FIG. 6).

Supernatants were subjected to downstream processing, involving an initial filter clarification followed by two TFF steps, cushion ultracentrifugation, another TFF, gradient ultracentrifugation, chromatography and eventually inactivation by UV irradiation.

The downstream processing was initiated with a first filter clarification step using filters with a pore size of 5 μM and 0.65 μM, followed by two TFF steps with two hollow fibers with different surface areas, which resulted in a volume reduction from ~16L to ~35 ml. The resulting material had HCV infectivity titers of 7.5, 9.2 and 8.8 log 10 FFU/ml for genotype 1a, 2a and 3a HCV, respectively.

The resulting material was distributed into six different ultracentrifugation vials, which were subjected to 3-cushion ultracentrifugation resulting in three fractions. Fraction 2 from each of the six vials (~1.2 ml each) were pooled amounting to a total volume of ~7.5 ml with HCV infectivity titers of 7.8, 9.5 and 9.1 log 10 FFU/ml for genotype 1a, 2a and 3a HCV, respectively.

This was followed by TFF using a small hollow fiber reducing the volume to ~2 ml with infectivity titers of 8.5, 11 and 9.6 log 10 FFU/ml for genotype 1a, 2a and 3a HCV, respectively. These samples were subjected to gradient ultracentrifugation; 18 fractions (~550 μl each) were collected and weighed to determine their buoyant densities. The three fractions with densities closest to 1.1 g/ml were pooled amounting to a total volume of ~1.5 ml. These pools yielded infectivity titers of 7.8, 9.9 and 9.6 log 10 FFU/ml for genotype 1a, 2a and 3a HCV, respectively. These pools were then subjected to Sephadex chromatography, where 12 fractions (~1 ml each) were collected. Five of these fractions were pooled based on absorbance at OD of 230 nm amounting to a total volume of ~4.8 ml with infectivity titers of 7.4, 9.2 and 9.1 log 10 FFU/ml for genotype 1a, 2a and 3a HCV, respectively.

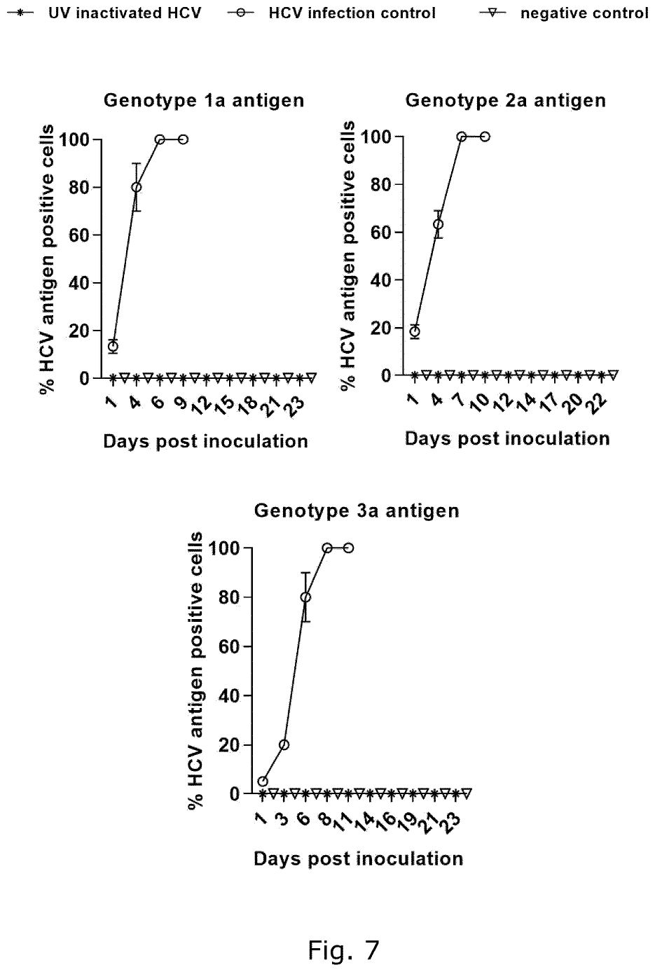

Thus, recoveries in downstream processing were 9%, 17% and 30% for genotype 1a, 2a and 3a HCV, respectively. The pools resulting from chromatography were UV inactivated to yield the final vaccine antigens. To confirm virus inactivation, replicate cell cultures were inoculated and maintained for three weeks; cells were split every 2-3 days, when replicate cultures for immunostaining for HCV antigen were plated, to confirm inactivation based on absence of HCV antigen positive cells (FIG. 7).

### Conclusion

Vaccine candidates were successfully produced for all three HCV genotypes.

### Example 7-Immunization of Mice with Inactivated Genotype 1a, 2a and 3a HCV Vaccine Candidates Elicited Broadly Neutralizing and Envelope-Protein Binding Antibodies

### Aim

To study immunogenicity of genotype 1a, 2a and 3a HCV vaccine candidates.

### Materials and Methods

See example 1.

### Results

Processed inactivated genotype 1a, 2a or 3a HCVcc or control antigen ovalbumin (OVA) were formulated with the adjuvant AddaVax, an analogue of the adjuvant MF-59, which is licensed for human use, and used for immunization of BALB/c mice.

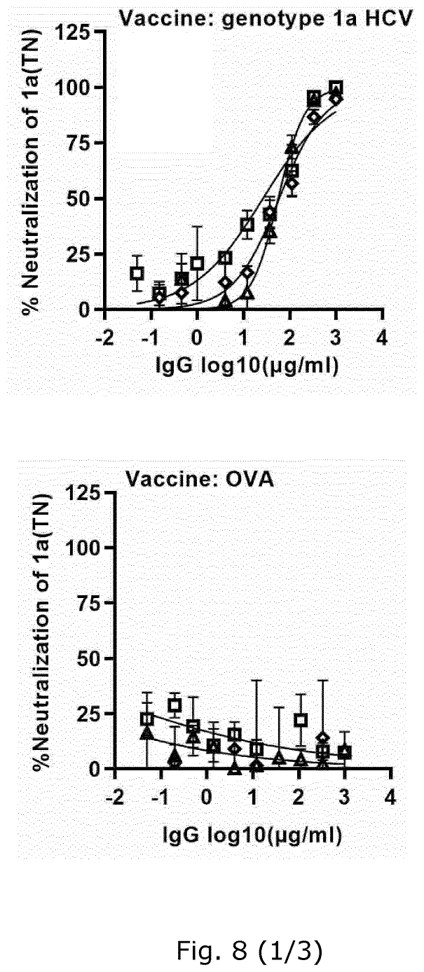

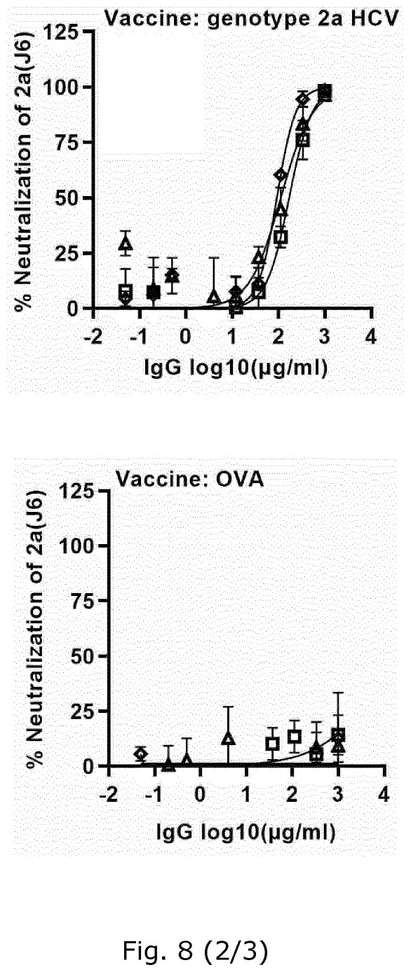

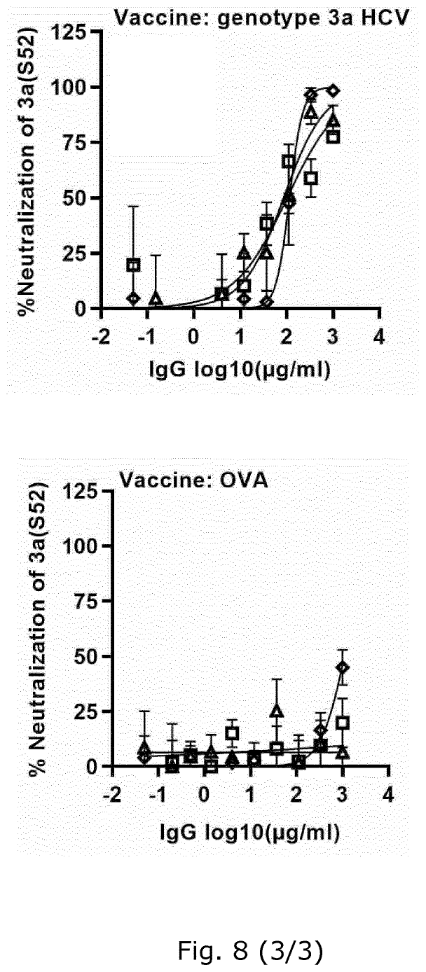

Purified serum IgG from individual animals neutralized HCV with in vivo derived envelope protein sequences of the same genotype as used in the respective vaccine in a concentration dependent manner, with mean EC50 of 47, 124 and 101 μg/ml for genotype 1a, 2a and 3a HCV vaccines, respectively (FIG. 8 and Table 10). Close to complete neutralization was achieved at the highest applied IgG concentration of 1000 μg/ml. OVA immunized mice did not elicit HCV nAb.

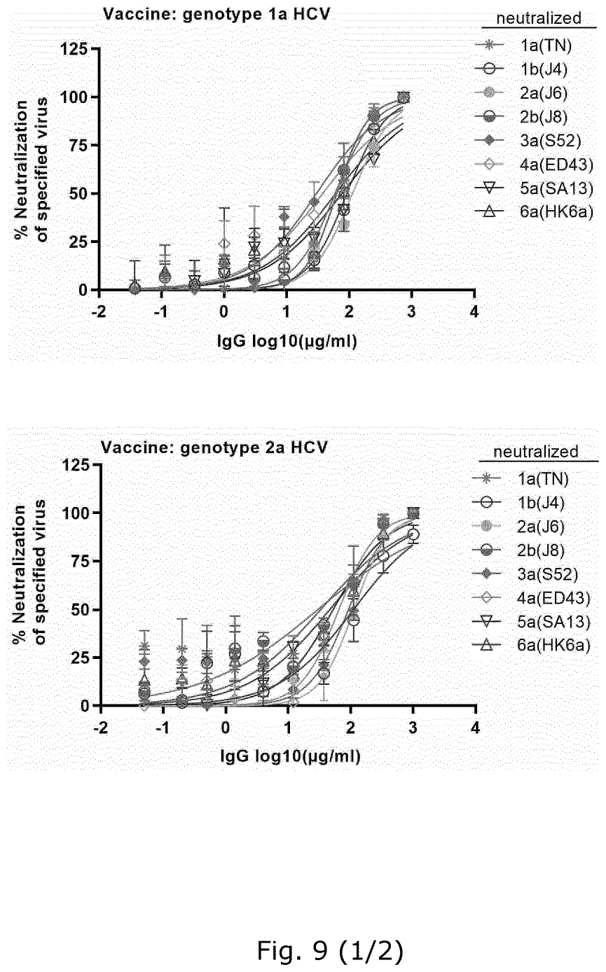

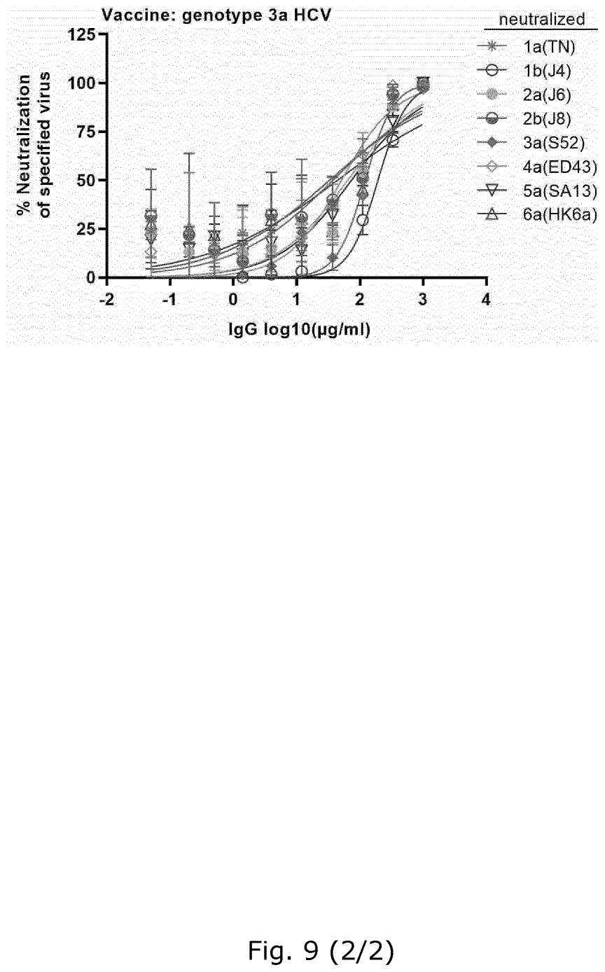

Moreover, IgG pools neutralized genotype 1-5 HCV with in vivo derived envelope sequences and genotype 6 HCV with 2 vital cell culture adaptive substitutions in the envelope proteins with similar efficacy and in a concentration dependent manner. Across neutralized viruses, mean EC50 of IgG from genotype 1a, 2a and 3a vaccinated animals were 67, 68 and 77 μg/ml, respectively, and close to complete neutralization was observed at 1000 μg/ml (FIG. 9 and Table 11).

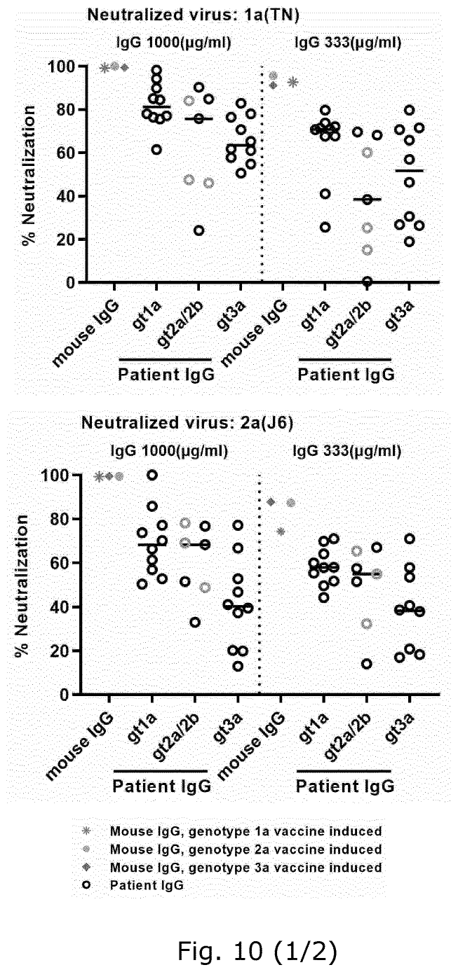

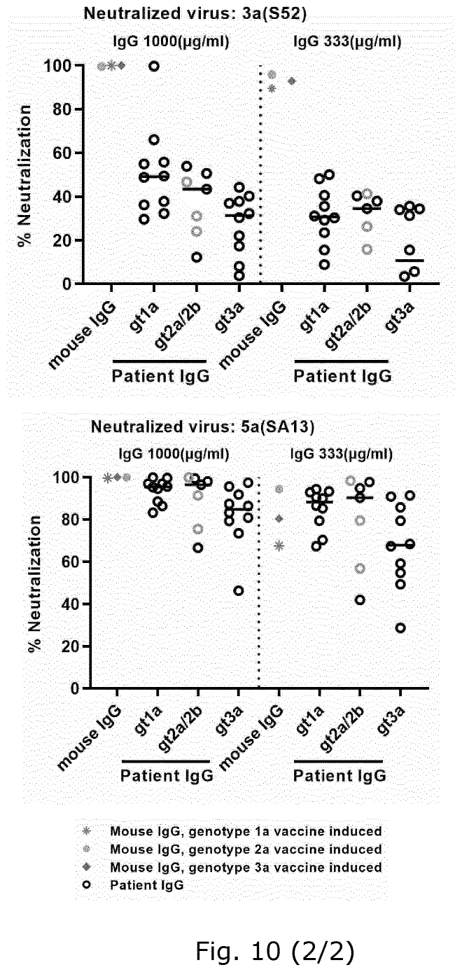

Mouse IgG neutralization capacity compared favorably to that of IgG from patients with CHC regarding efficacy and broadness (FIG. 10). Interestingly, these data confirmed that 5a (SA13) had relatively high neutralization sensitivity, while 2a (J6) and 3a (S52) had relatively low neutralization sensitivity (Bankwitz et al., 2021). Further, neutralization capacity of IgG from genotype 3a infected patients was lower than that from genotype 1a, 2a or 2b infected patients.

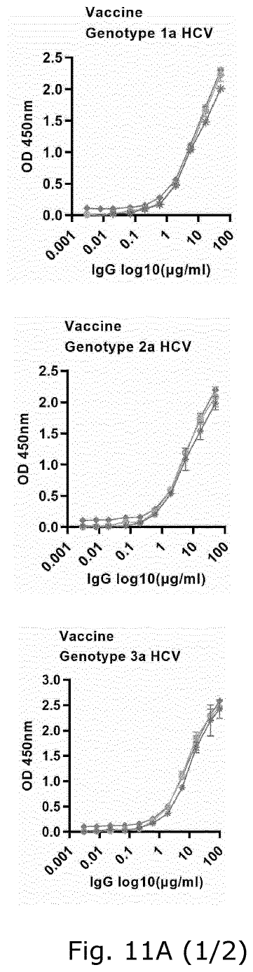

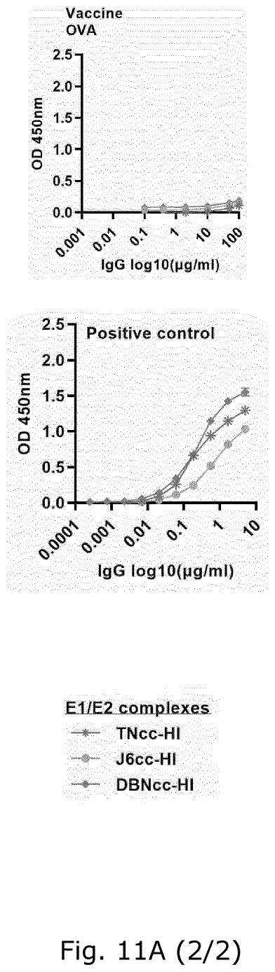

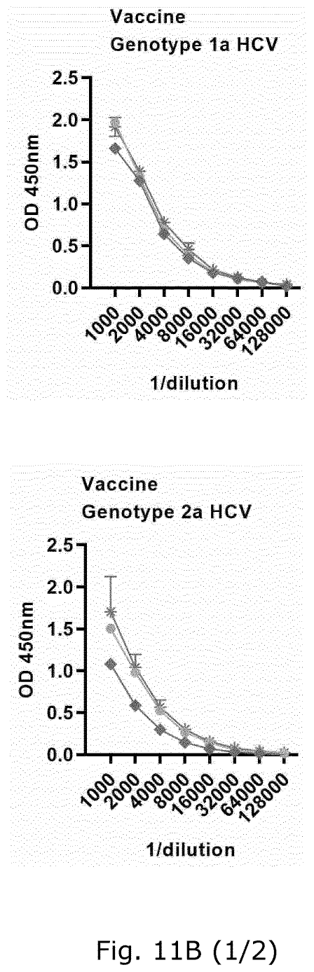

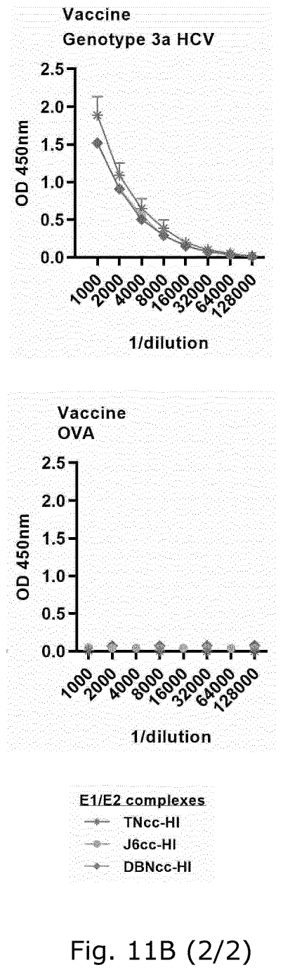

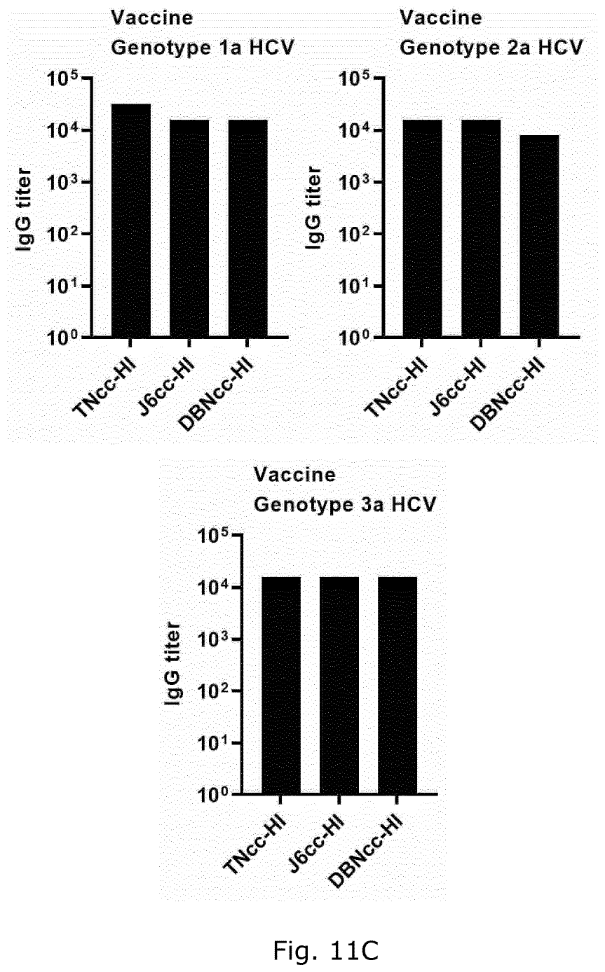

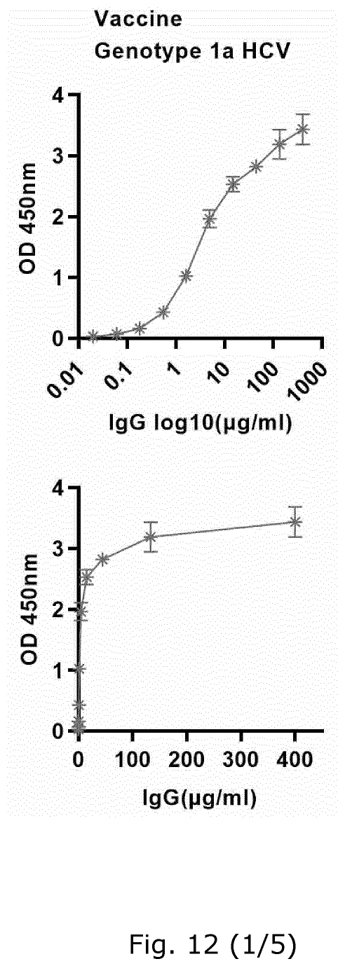

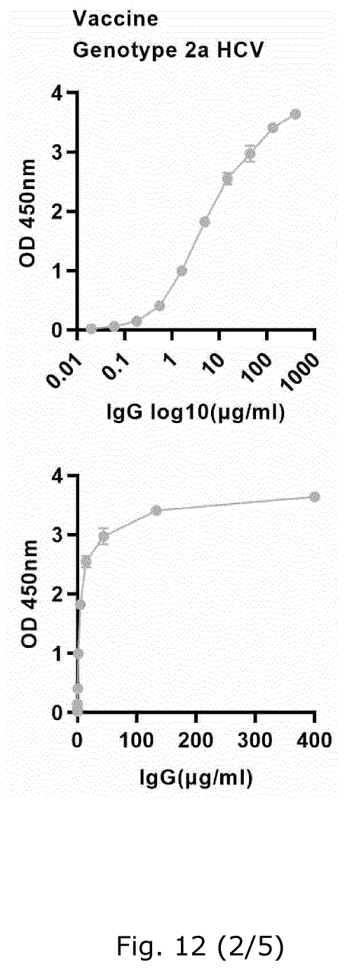

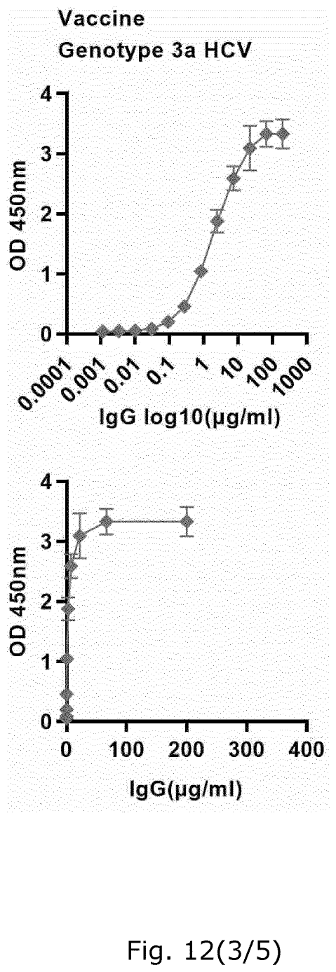

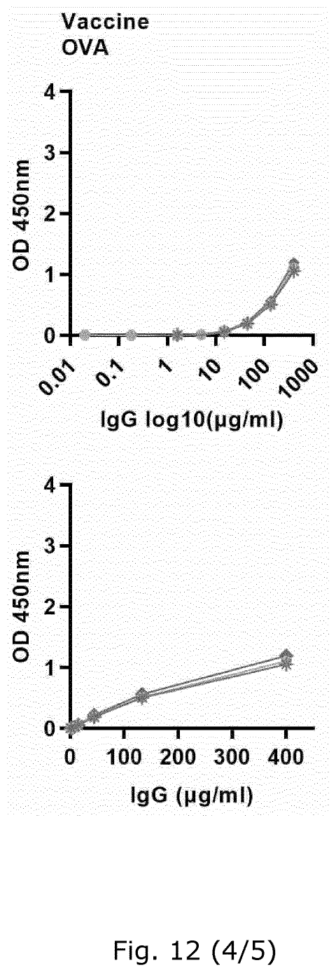

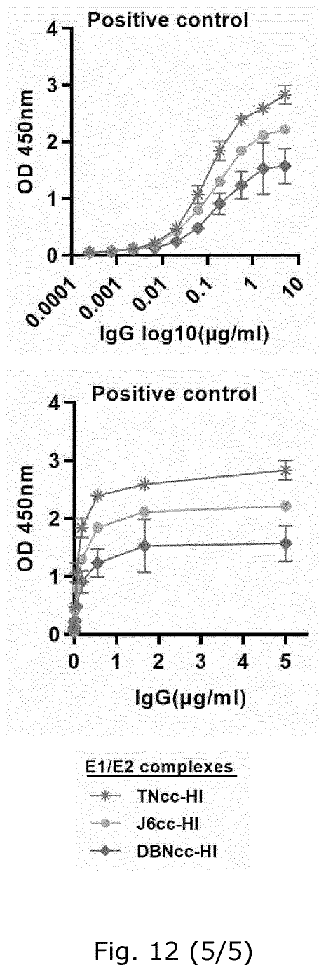

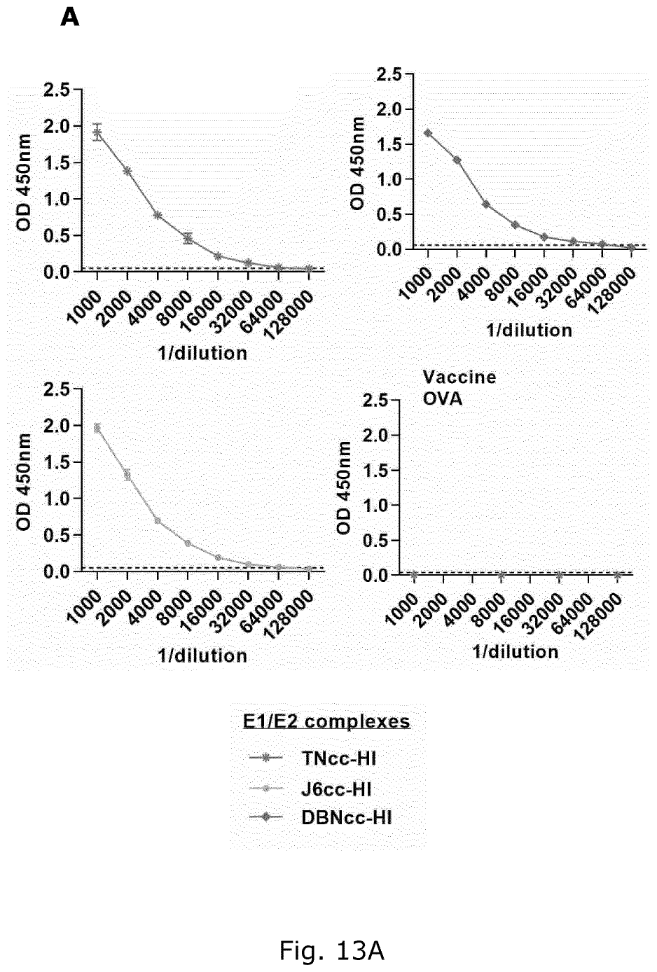

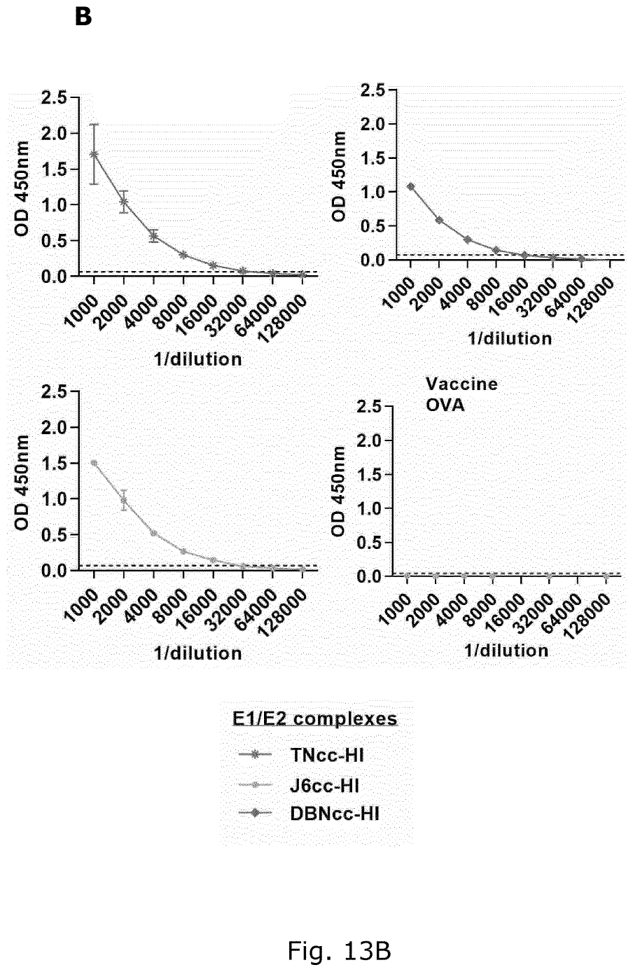

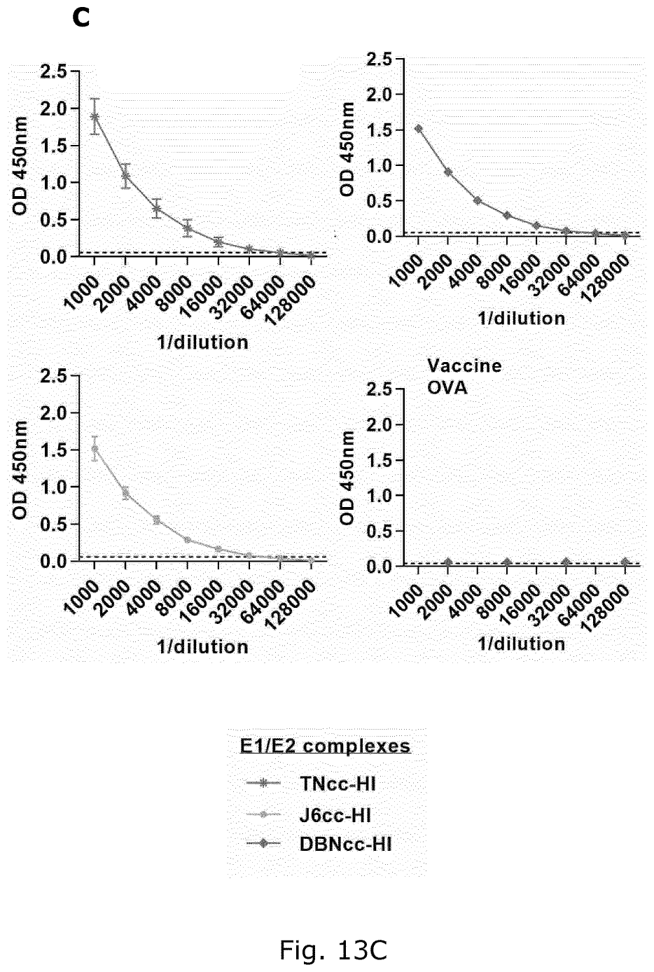

Finally, IgG pools from all HCV immunized animal groups efficiently bound to TNcc-HI, J6cc-HI and DBNcc-HI E1/E2 complexes in a concentration-dependent manner (FIG. 11-12). Such binding was not observed for pooled IgG from OVA immunized animals. Moreover, pools of immune-sera had endpoint titers of up to 32,000 (FIGS. 11 and 13).

### Discussion

High-yield genotype 1a, 2a and 3a HCV cell culture systems were developed to facilitate development of whole virus inactivated vaccine candidates. Compared to the original viruses, high-yield viruses showed increased exposure of conserved conformational neutralizing epitopes associated with protection against chronic HCV infection, as suggested by increased sensitivity to neutralization by mAb AR3A and AR4A. In mouse immunogenicity studies, high-yield genotype 1a, 2a or 3a viruses, formulated with an analogue of the human MF-59 adjuvant, each had the capacity to induce efficient nAb broadly neutralizing HCV of all major genotypes with recognized epidemiological importance.

For efficient production of whole virus inactivated vaccines high-yield virus production systems are required. The developed genetically stable, high-yield HCV recombinants can in the future be used to initiate virus vaccine antigen production with sequence confirmed early-viral-passage seed stocks. Based on results from this study using full-length recombinants, as well as on results from a previous study using a JFH1-based recombinant (Mathiesen et al., 2015), it appears that the upper limit for HCV infectivity titers in Huh7.5 cells grown in monolayers in cell culture flasks is between 6 and 7 log 10 FFU/ml, which might be due to limited availability of required host cell factors.

TN-PP-18 and TN-PP-38.1 acquired N410K in hypervariable region 1 (HVR1), while TN-PP-38.1 in addition acquired F403L in HVR1. J6cc-PP-35 acquired H434N in the E2 front layer and DBNcc-PP-16 acquired G395R in HVR1, while DBN3acc harbored S449A in the E2 front layer (Ramirez et al., 2016). TN-PP-38.1 acquired V719I and J6cc-PP-35 acquired A575T, while DBN3acc harbored D474A, T528N and V629A (Ramirez et al., 2016).

For HCV, in vivo protection of conserved conformational neutralizing epitopes might be associated with a fitness cost as closed E1/E2 states might decrease access of the main HCV entry receptor CD81 to its binding site, which is overlapping with AR3.

For HCV, deletion of HVR1 led to a maximally open E1/E2 state associated with high neutralization sensitivity. For the HI-viruses the AR3A epitopes were as accessible as in HVR1-deleted viruses, while the AR4A epitopes were approximately 10- to 100-fold less exposed (Prentoe et al., 2016). However, HVR1-deleted viruses typically show relatively low infectivity titers, hampering vaccine production. Compared to the original viruses, HI-viruses showed approximately 300- to 1250-fold higher exposure of AR3A epitopes and 12- to 2400-fold higher exposure of AR4A epitopes.

Therefore, the HI-viruses present interesting vaccine antigens, as they might facilitate induction of antibodies targeting epitopes that are conserved between HCV variants and that are mediating protection in humans. A vaccine antigen exposing such conserved epitopes with the ability to induce broadly nAb might make a multivalent vaccine approach unnecessary.

Indeed, immunization with PP- and HI-viruses resulted in induction of broadly nAb. 50% neutralization titers and ELISA endpoint titers of vaccine induced antibodies were comparable to those reported for licensed antiviral vaccines and to those in chimpanzees protected from HCV challenge following vaccination with the E1/E2 heterodimer vaccine. However, compared to IgG from chronically infected patients, antibodies elicited by the E1/E2 heterodimer vaccine and different vaccine candidates based on soluble E2 protein, nAb induced in this study showed increased capacity to neutralize different HCV variants.

### Conclusion

High-yield genotype 1a, 2a and 3a HCV were developed constituting a basis for inactivated vaccine candidates that could be used for further preclinical and clinical development.

TABLE 1
Primers for reverse transcription for generation of full-length HCV ORF
amplicons.

| 3′ cDNA | | | SEQ ID |
|---|---|---|---|
| primers | Target virus | Primer sequence (5′-3′) | NO: |
| 1a-9405-RT | 1a(TNcc, TNcc-HI) | TAAGAGGCCGGAGTGTTTAC | 13 |
| 2a-9481-RT | 2a(J6cc, J6cc-HI) | CTATGGAGTGTACCTAGTGTGTGC | 14 |
| 3a-9235-RT | 3a(DBNcc, DBNcc-HI) | AAAAGAATGGAGTGTTATC | 15 |

TABLE 2
Primers for PCR for generation of full-length HCV ORF amplicons and
amplicons spanning HCV E1/E2.

| | | Primer sequence (5′-3′) | SEQ ID |
|---|---|---|---|
| Primers | Target virus | | NO: |
| 1a-209-F | 1a(TNcc, TNcc-HI) | TGCCTGATAGGGTGCTTGCG | 16 |
| 1a-9402-R | 1a(TNcc, TNcc-HI) | AGGCCGGAGTGTTTACCCCA | 17 |
| 1a-3285R | 1a(TNcc, TNcc-HI) | TGGTCTCCATCTGGGAAAAG | 18 |
| 2a-303-F | 2a(J6cc, J6cc-HI) | CTTGCGAGTGCCCCGGGAGG | 19 |
| 2a-9467-R | 2a(J6cc, J6cc-HI) | TGGAGTGTACCTAGTGTGTGCCGCTC | 20 |
| 2a-3774-R | 2a(J6cc, J6cc-HI) | GGGATGACATCAGCGTTCCGCGTGAC | 21 |
| 3a-293-F | 3a(DBNcc, DBNcc-HI) | GATAGGGTGCTTGCGAGTGCC | 22 |
| 3a-9432-R | 3a(DBNcc, DBNcc-HI) | AGAATGGAGTGTTATCCTACCAGCTCA | 23 |
| 3a-3694R | 3a(DBNcc, DBNcc-HI) | CTGGCCACCCAACRAGRTCCT | 24 |

TABLE 3
Coding nucleotide changes identified in polyclonal passaged genotype 1a HCV from an initial passage line.

| | Nucleotide change^{b} | | | Allele frequency (%)^{c} | | | | | |
|---|---|---|---|---|---|---|---|---|---|
| HCV | TNcc | TNcc | | | | Gt 1a HCV | AA change | Engineered construct^{e} | |
| proteina | position | reference | change | TNcc-PP-10 | TNcc-PP-18 | seed stock | TNcc^{d} | TNcc-HI-18A | TNcc-HI-18B |
| Core | 373 | C | A | 16 | 26 | 41 | T11N | | |
| | 430 | T | C | 7 | 12 | 11 | I30T | | |
| E2 | 1571 | C | G | 18 | 27 | 42 | N410K | | |
| | 2464 | G | A | 11 | 29 | 16 | S708N | | |
| NS2 | 2822 | G | A | 5 | 16 | 24 | M827I | | |
| | 2895 | A | G | 17 | 30 | 15 | R852G | | |
| | 2935 | A | C | 1 | 14 | 21 | N865T | | |
| | 3364 | G | A | 18 | 28 | 44 | R1008Q | | |
| NS3 | 3804 | C | G | 20 | 33 | 15 | P1155A | | |
| | 3978 | A | T | 2 | 10 | 23 | N1213Y | | |
| | 4848 | G | A | 5 | 10 | 10 | A1503T | | |
| NS4B | 5812 | G | A | 15 | 5 | 12 | G1824D | | |
| | 6067 | G | C | 25 | 36 | 59 | G1909A | | |
| NS5A | 6729 | C | A | 4 | 24 | 40 | L2130I | | |
| | 7296 | C | T | 5 | 15 | 23 | P2319S | | |
| | 7410 | A | G | 24 | 34 | 17 | T2357A | | x |
| | 7464 | A | G | 29 | 45 | 27 | S2375G | | x |
| | 7588 | A | G | 9 | 20 | 23 | D2416G | | |
| | 7591 | T | C | 32 | 34 | 46 | V2417A | | |
| | 7596 | T | C | 25 | 43 | 29 | C2419R | | x |
| NS5B | 7785 | A | G | 18 | 29 | 43 | S2482G | x | |
| | 8292 | A | C | 61 | 60 | 80 | N2651H | x | |
| | 8841 | A | T | 29 | 38 | 20 | M2834L | | x |
| | 8901 | A | C | 5 | 11 | 11 | I2854L | | |
| | 8985 | A | C | 9 | 18 | 24 | I2882L | | |
| | 9045 | A | C | 7 | 27 | 43 | I2902L | x | |
| | 9298 | A | G | 72 | 96 | 98 | H2986R | x | x |

^{a}HCV protein, in which specified change was located.
^{b}Nucleotide change specified by nucleotide position and identity relating to the TNcc genome (Genbank accession number JX993348) as a reference.²⁶ Coding changes with allele frequency of at least 10% in one of the analyzed samples are listed.
^{c}Allele frequency of the identified nucleotide change. Polyclonal passage 10 (TNcc-PP-10) and 18 (TNcc-PP-18) virus from the initial passage line (FIG. 1), and the seed stock used for vaccine production (Gt 1a HCV seed stock) were analyzed.
^{d}Amino acid change specified by amino acid position and identity relating to the TNcc polyprotein; amino acid position numbers are identical to position numbers in relation to the reference H77 polyprotein (Genbank accession number AF009606).
^{e}x indicates that the respective nucleotide change was engineered for generation of TNcc-HI-18A and TNcc-HI-18B, respectively.

TABLE 4
Coding nucleotide changes identified in polyclonal
passaged genotype 1a HCV from a later passage line.

| | | | | Allele | | |
|---|---|---|---|---|---|---|
| | Nucleotide change^{b} | | | frequency | | Engineered |
| HCV | TNcc | TNcc | | (%)^{c} | AA change | construc^{e} |
| protein^{a} | position | reference | change | TNcc-PP-38.1 | TNcc ^{d} | TNcc-HI |
| Core | 373 | C | A | 97 | T11N | x |
| | 435 | G | A | 48 | G32S | x |
| | 756 | C | A | 17 | L139I | |
| E1 | 1435 | T | C | 99 | V365A | x |
| E2 | 1548 | T | C | 99 | F403L | x |
| | 1571 | C | G | 100 | N410K | x |
| | 2496 | G | A | 100 | V719I | x |
| p7 | 2701 | T | C | 92 | V787A | x |
| NS2 | 2917 | T | C | 100 | V859A | x |
| | 3364 | G | A | 100 | R1008Q | x |
| NS3 | 4267 | C | T | 15 | A1309V | |
| | 4363 | C | T | 100 | A1341V | x |
| | 4711 | G | C | 99 | C1457S | x |
| NS4B | 6067 | G | C | 100 | G1909A | x |
| NS5A | 6729 | C | A | 100 | L2130I | x |
| | 7591 | T | C | 100 | V2417A | x |
| NS5B | 7785 | A | G | 99 | S2482G | x |
| | 8292 | A | C | 99 | N2651H | x |
| | 9045 | A | C | 100 | I2902L | x |
| | 9298 | A | G | 99 | H2986R | x |

^{a}HCV protein in which specified change was located.
^{b}Nucleotide change specified by nucleotide position and identity relating to the TNcc genome (Genbank accession number JX993348) as a reference.²⁶ Coding changes with allele frequency of at least 10% in one of the analyzed samples are listed.
^{c}Allele frequency of the identified nucleotide change. Polyclonal passage 38.1 (TNcc-PP-38.1) virus from the later passage line was analyzed (FIG. 1).
^{d} Amino acid change specified by amino acid position and identity relating to the TNcc polyprotein; amino acid position numbers are identical to position numbers in relation to the reference H77 polyprotein (Genbank accession number AF009606).
^{e}x indicates that the respective nucleotide change was engineered for generation of TNcc-HI.

TABLE 5
Coding nucleotide changes identified in polyclonal passaged genotype 2a HCV.

| | Nucleotide change^{b} | | | | | AA change | AA position | Engineered |
|---|---|---|---|---|---|---|---|---|
| HCV | J6cc | J6cc | | Allele frequency (%)^{c} | | J6cc | H77 reference | construct^{f} |
| Protein^{a} | position | reference | change | J6cc-PP-35 | Gt 2a HCV seed stock | reference^{d} | number^{e} | J6cc-HI |
| core | 572 | A | G | 99 | 99 | K78E | 78 | x |
| | 758 | G | C | 81 | 81 | V140L | 140 | x |
| E1 | 1325 | A | T | 100 | 100 | T329S | 329 | x |
| E2 | 1583 | A | G | 14 | 14 | N415D | 415 | |
| | 1640 | C | A | 100 | 100 | H434N | 434 | x |
| | 2063 | G | A | 83 | 83 | A575T | 573 | x |
| p7 | 2618 | G | A | 94 | 94 | A760T | 756 | x |
| | 2658 | T | C | 94 | 94 | V773A | 769 | x |
| NS2 | 2823 | T | C | 93 | 93 | V828A | 824 | x |
| | 2913 | A | C | 17 | 17 | E858A | 854 | |
| NS3 | 3777 | C | T | 82 | 82 | A1146V | 1142 | x |
| | 4328 | A | G | 100 | 100 | I1330V | 1326 | x |
| | 4839 | T | C | 93 | 93 | L1500P | 1496 | x |
| NS4B | 5843 | G | A | 82 | 82 | V1835I | 1831 | x |
| NS5A | 6338 | A | G | 93 | 93 | T2000A | 1996 | x |
| | 7428 | T | C | 100 | 92 | L2363P | 2363 | x |
| | 7439 | G | C | 100 | 100 | A2367P | 2367 | x |
| | 7661 | T | C | 45 | 45 | C2441R | 2419 | |
| NS5B | 8687 | G | C | 81 | 81 | E2783Q | 2761 | x |
| | 9378 | T | C | 95 | 45 | L3013S | 2991 | x |

^{a}HCV protein in which specified change was located.
^{b}Nucleotide change specified by nucleotide position and identity relating to the J6cc genome (Genbank accession number JQ745650) as a reference (Li et al., 2012b). Coding changes with allele frequency of at least 10% in one of the analyzed samples are listed.
^{c}Allele frequency of the identified nucleotide change. Polyclonal passage 35 (J6cc-PP-35) virus (FIG. 1) and the seed stock used for vaccine production (Gt 2a HCV seed stock) were analyzed.
^{d}Amino acid change specified by amino acid position and identity relating to the J6cc polyprotein (Genbank accession number JQ745650).
^{e}Amino acid position relating to the H77 reference polyprotein (Genbank accession number AF009606).
^{f}x indicates that the respective nucleotide change was engineered for generation of J6cc-HI.

TABLE 6
Coding nucleotide changes identified in polyclonal passaged genotype 3a HCV.

| | | | | | | | AA position | |
|---|---|---|---|---|---|---|---|---|
| | Nucleotide change^{b} | | | Allele frequency (%)^{c} | | AA change | H77 | Engineered |
| HCV | DBN3acc | DBN3acc | | | Gt 3a HCV | DBN3acc | reference | construct^{f} |
| protein^{a} | position | reference | change | DBNcc-PP-16 | seed stock | reference^{d} | number^{e} | DBNcc-HI |
| E2 | 1522 | G | A | 97 | 100 | G395R | 395 | x |
| NS2 | 2905 | G | A | 96 | 100 | A856T | 849 | x |
| | 2962 | G | A | 97 | 100 | G875R | 868 | x |
| NS3 | 5105 | A | G | 97 | 100 | N1589S | 1582 | x |
| NS4B | 5597 | T | G | 96 | 100 | I1753S | 1746 | x |
| NS5A | 7622 | T | C | 97 | 100 | V2428A | 2417 | x |
| NS5B | 8759 | A | G | 97 | 100 | D2807G | 2796 | x |

^{a}HCV protein in which specified change was located.
^{b}Nucleotide change specified by nucleotide position and identity relating to the DBN3acc genome (Genbank accession number KX280714) as a reference.²⁸ Coding changes with allele frequency of at least 10% in one of the analyzed samples are listed.
^{c}Allele frequency of the identified nucleotide change. Polyclonal passage 16 (DBNcc-PP-16) virus (FIG. 1) and the seed stock used for vaccine production (Gt 3a HCV seed stock) were analyzed.
^{d}Amino acid change specified by amino acid position and identity relating to the DBN3acc polyprotein (Genbank accession number KX280714).
^{e}Amino acid position relating to the H77 reference polyprotein (Genbank accession number AF009606).
^{f}x indicates that the respective nucleotide change was engineered for generation

TABLE 7
EC50 of human antibodies AR3A, AR4A and C211 in neutralization
assays employing original and high-yield HCV recombinants,
calculated based on curves in FIG. 5. Fold, fold increased
neutralization sensitivity, calculated as [(EC50 of original
virus)/(EC50 of virus with envelope substitutions)].

| | AR3A | | AR4A | | C211 | |
|---|---|---|---|---|---|---|
| | EC50 | fold | EC50 | fold | EC50 | fold |
| 1a(TN) | 0.3 | na | 24 | na | 22 | na |
| TNcc-HI | 0.001 | 300 | 0.01 | 2400 | 0.2 | 110 |
| Gt 1a HCV | 0.1 | 3 | 1.6 | 15 | 6.8 | 3.2 |
| seed | | | | | | |
| stock | | | | | | |

TABLE 8
EC50 of human antibodies AR3A, AR4A and C211 in neutralization
assays employing original and high-yield HCV recombinants,
calculated based on curves in FIG. 5. Fold, fold increased
neutralization sensitivity, calculated as [(EC50 of original
virus)/(EC50 of virus with envelope substitutions)].

| | | |
|---|---|---|
| AR3A | AR4A | C211 |

| | EC50 | fold | EC50 | fold | EC50 | fold |
|---|---|---|---|---|---|---|
| 2a(J6) | 4.4 | na | 1.1 | na | 57 | na |
| J6cc-HI | 0.01 | 440 | 0.09 | 12 | 0.09 | 633 |

TABLE 9
EC50 of human antibodies AR3A, AR4A and C211 in neutralization
assays employing original and high-yield HCV recombinants,
calculated based on curves in FIG. 5. Fold, fold increased
neutralization sensitivity, calculated as [(EC50 of original
virus)/(EC50 of virus with envelope substitutions)].

| | AR3A | | AR4A | | C211 | |
|---|---|---|---|---|---|---|
| | EC50 | fold | EC50 | fold | EC50 | fold |
| 3a(DBN) | 10 | na | 2.2 | na | 1236 | na |
| DBNcc-HI | 0.008 | 1250 | 0.01 | 220 | 0.5 | 2472 |
| DBN3acc | 0.06 | 167 | 0.1 | 22 | 14 | 88 |

TABLE 10
EC50 of purified mouse serum IgG induced by HCV genotype 1a,
2a and 3a vaccines in neutralization assays employing HCV
recombinants of the same genotype, calculated based on curves
in FIG. 8. Each vaccine was used to immunize 3 mice.

| | | EC₅₀ (Vaccine: same genotype) | | |
|---|---|---|---|---|
| Neutralized | Vaccine | Mouse 1 | Mouse 2 | Mouse 3 |
| 1a(TN) | HCV genotype 1a | 60 | 56 | 26 |
| 2a(J6) | HCV genotype 2a | 92 | 112 | 169 |
| 3a(S52) | HCV genotype 3a | 115 | 86 | 103 |

TABLE 11
EC50 of purified mouse serum IgG induced by HCV genotype 1a,
2a and 3a vaccines in neutralization assays employing HCV
recombinants of the indicated genotypes(isolates), calculated
based on curves in FIG. 9. Prior to neutralization assays,
IgG from mice immunized with the same vaccine was pooled.

| | EC₅₀ | | |
|---|---|---|---|
| | 1a HCV | 2a HCV | 3a HCV |
| Neutralized | vaccine | vaccine | vaccine |
| 1a(TN) | 60 | 30 | 38 |
| 1b(J4) | 92 | 111 | 193 |
| 2a(J6) | 113 | 113 | 64 |
| 2b(J8) | 63 | 33 | 33 |
| 3a(S52) | 30 | 92 | 121 |
| 4a(ED43) | 38 | 65 | 51 |
| 5a(SA13) | 77 | 52 | 74 |
| 6a(HK6a) | 61 | 46 | 45 |

### REFERENCES

Akazawa D, Moriyama M, Yokokawa H, et al. Neutralizing antibodies induced by cell culture-derived hepatitis C virus protect against infection in mice. Gastroenterology. 2013; 145.
Altschul et al. (1997). Gapped BLAST and PSI-BLAST: a new generation of protein database search programs. Nucleic acids research 25, 3389-3402; 10.1093/nar/25.17.3389.
Altschul et al. (2005). Protein database searches using compositionally adjusted substitution matrices. The FEBS journal 272, 5101-5109; 10.1111/j.1742-4658.2005.04945.x.
Augestad E H, Castelli M, Clementi N, et al. Global and local envelope protein dynamics of hepatitis C virus determine broad antibody sensitivity. Sci Adv. 2020; 6: eabb5938.
Bankwitz D, Bahai A, Labuhn M, et al. Hepatitis C reference viruses highlight potent antibody responses and diverse viral functional interactions with neutralising antibodies. Gut 2021; 70:1734-1745.
Carlsen T H R, Pedersen J, Prentoe J C, et al. Breadth of neutralization and synergy of clinically relevant human monoclonal antibodies against HCV genotypes 1a, 1b, 2a, 2b, 2c, and 3a. Hepatology. 2014; 60:1551-1562.
Clayton R F, Owsianka A, Aitken J, Graham S, Bhella D, Patel A H. Analysis of Antigenicity and Topology of E2 Glycoprotein Present on Recombinant Hepatitis C Virus-Like Particles. J Virol. 2002; 76:7672-7682.
Czarnota A, Offersgaard A, Pihl A F, et al. Specific antibodies induced by immunization with hepatitis B virus-like particles carrying hepatitis C virus envelope glycoprotein 2 epitopes show differential neutralization efficiency. Vaccines. 2020; 8:1-19.
Giang E, Dorner M, Prentoe J C, et al. Human broadly neutralizing antibodies to the envelope glycoprotein complex of hepatitis C virus. Proc Natl Acad Sci USA. 2012; 109:6205-6210.
Gottwein J M, Scheel T K H, Jensen T B, et al. Development and characterization of hepatitis C virus genotype 1-7 cell culture systems: Role of CD81 and scavenger receptor class B type I and effect of antiviral drugs. Hepatology. 2009; 49:364-377.
Jensen S B, Fahnøe U, Pham L V., et al. Evolutionary Pathways to Persistence of Highly Fit and Resistant Hepatitis C Virus Protease Inhibitor Escape Variants. Hepatology. 2019; 70:771-787.
Law M, Maruyama T, Lewis J, et al. Broadly neutralizing antibodies protect against hepatitis C virus quasispecies challenge. Nat Med. 2008; 14:25-27.
Li Y P, Ramirez S, Gottwein J M, et al. Robust full-length hepatitis C virus genotype 2a and 2b infectious cultures using mutations identified by a systematic approach applicable to patient strains. Proc Natl Acad Sci USA. 2012 (b); 109:6806.
Li Y P, Ramirez S, Jensen S B, Purcell R H, Gottwein J M, Bukh J. Highly efficient full-length hepatitis C virus genotype 1 (strain TN) infectious culture system. Proc Natl Acad Sci USA. 2012 (a); 109:19757-19762.
Lindenbach B D, Evans M J, Syder A J, et al. Virology: Complete replication of hepatitis C virus in cell culture. Science. 2005; 309:623-626.
Lothert K, Offersgaard A F, Pihl A F, et al. Development of a downstream process for the production of an inactivated whole hepatitis C virus vaccine. Sci Rep. 2020; 10:1-13.
Mathiesen C K, Jensen T B, Prentoe J, et al. Production and characterization of high-titer serum-free cell culture grown hepatitis C virus particles of genotype 1-6. Virology. 2014; 458-459:190-208.
Mathiesen C K, Prentoe J, Meredith L W, et al. Adaptive Mutations Enhance Assembly and Cell-to-Cell Transmission of a High-Titer Hepatitis C Virus Genotype 5a Core-NS2 JFH1-Based Recombinant. J Virol. 2015; 89:7758-7775.
Pham L V., Jensen S B, Fahnøe U, et al. HCV genotype 1-6 NS3 residue 80 substitutions impact protease inhibitor activity and promote viral escape. J Hepatol. 2019; 70:388-397.
Pihl A F, Feng S, Offersgaard A, et al. Inactivated whole hepatitis C virus vaccine employing a licensed adjuvant elicits cross-genotype neutralizing antibodies in mice. J Hepatol. 2022; 76:1051-1061.
Prentoe J, Velázquez-Moctezuma R, Augestad E H, et al. Hypervariable region 1 and N-linked glycans of hepatitis C regulate virion neutralization by modulating envelope conformations. Proc Natl Acad Sci USA. 2019; 116:10039-10047.
Prentoe J, Velázquez-Moctezuma R, Foung S K H, Law M, Bukh J. Hypervariable region 1 shielding of hepatitis C virus is a main contributor to genotypic differences in neutralization sensitivity. Hepatology. 2016; 64:1881-1892.
Ramirez S, Mikkelsen L S, Gottwein J M, Bukh J. Robust HCV Genotype 3a Infectious Cell Culture System Permits Identification of Escape Variants With Resistance to Sofosbuvir. Gastroenterology. 2016; 151:973-985.
Sabo M C, Luca V C, Prentoe J, et al. Neutralizing Monoclonal Antibodies against Hepatitis C Virus E2 Protein Bind Discontinuous Epitopes and Inhibit Infection at a Postattachment Step. J Virol. 2011; 85:7005-7019.
Scheel T K H, Gottwein J M, Carlsen T H R, et al. Efficient Culture Adaptation of Hepatitis C Virus Recombinants with Genotype-Specific Core-NS2 by Using Previously Identified Mutations. J Virol. 2011; 85:2891-2906.
Yokokawa H, Higashino A, Suzuki S, et al. Induction of humoural and cellular immunity by immunisation with HCV particle vaccine in a non-human primate model. Gut. 2018; 67:372-379.

Sequence listing

| SEQ | | | |
|---|---|---|---|
| ID NO: | Name | AA/NA | GenBank |
| 1 | H77 | AA | AF009606 |
| 2 | TNcc-HI-18A | AA | |
| 3 | TNcc-HI-18B | AA | |
| 4 | TNcc-HI | AA | |
| 5 | J6cc-HI | AA | |
| 6 | DBNcc-HI | AA | |
| 7 | TNcc-HI-18A | NA | |
| 8 | TNcc-HI-18B | NA | |
| 9 | TNcc-HI | NA | |
| 10 | J6cc-HI | NA | |
| 11 | DBNcc-HI | NA | |
| 12 | H77 | NA | AF009606 |
| 13 | Primer (1a-9405-RT) | | |
| 14 | Primer (2a-9481-RT) | | |
| 15 | Primer (3a-9235-RT) | | |
| 16 | Primer (1a-209-F) | | |
| 17 | Primer (1a-9402-R) | | |
| 18 | Primer (1a-3285-R) | | |
| 19 | Primer (2a-303-F) | | |
| 20 | Primer (2a-9467-R) | | |
| 21 | Primer (2a-3774-R) | | |
| 22 | Primer (3a-293-F) | | |
| 23 | Primer (3a-9432-R) | | |
| 24 | Primer (3a-3694-R) | | |
| 25 | TNcc | AA | JX993348 |
| 26 | J6cc | AA | JQ745650 |
| 27 | DBN3acc | AA | KX280714 |
| 28 | TNcc | NA | JX993348 |
| 29 | J6cc | NA | JQ745650 |
| 30 | DBN3acc | NA | KX280714 |

1. An isolated nucleic acid molecule which encodes a human hepatitis C virus or a fragment hereof,
wherein when said hepatitis C virus is derived from genotype 1a, said nucleic acid molecule encodes an amino acid sequence comprising one of the following groups of adaptive mutations:
a) S2482G, N2651H, I2902L and H2986R according to SEQ ID NO: 25; or
b) T2357A, S2375G, C2419R, M2834L and H2986R according to SEQ ID NO: 25;
wherein when said hepatitis C virus is derived from genotype 2a, said nucleic acid molecule encodes an amino acid sequence comprising the following adaptive mutations: K78E, V140L, T329S, H434N, A575T, A760T, V773A, V828A, A1146V, I1330V, L1500P, V1835I, T2000A, L2363P, A2367P, E2783Q and L3013S according to SEQ ID NO: 26; or
wherein when said hepatitis C virus is derived from genotype 3a, said nucleic acid molecule encodes an amino acid sequence comprising the following adaptive mutations: G395R, A856T, G875R, N1589S, 11753S, V2428A and D2807G according to SEQ ID NO: 27.

2-15. (canceled)

16. The isolated nucleic acid molecule according to claim 1, wherein
the hepatitis C virus is derived from genotype 1a, strain TNcc;
the hepatitis C virus is derived from genotype 2a, strain J6cc; or
the hepatitis C virus is derived from genotype 3a, strain DBN3acc.

17. The isolated nucleic acid molecule according to claim 1, wherein
said nucleic acid molecule encodes an amino acid sequence with a sequence identity of at least 95% to that of SEQ ID NO: 25;
said nucleic acid molecule encodes an amino acid sequence with a sequence identity of at least 95% to that of SEQ ID NO: 26; or
said nucleic acid molecule encodes an amino acid sequence with a sequence identity of at least 95% to that of SEQ ID NO: 27.

18. The isolated nucleic acid molecule according to claim 1, wherein said nucleic acid molecule encodes an amino acid sequence further comprising the following adaptive mutations: T11N, G32S, V365A, F403L, N410K, V719I, V787A, V859A, R1008Q, A1341V, C1457S, G1909A, L2130I and V2417A according to SEQ ID NO: 25, wherein said nucleic acid molecule encodes an amino acid sequence derived from genotype 1a and said adaptive mutations of group a).

19. The isolated nucleic acid molecule according to claim 1, wherein said nucleic acid molecule encodes an amino acid sequence further comprising the following adaptive mutations: T11N, G32S, L179P, V365A, F403L, N410K, V719I, V787A, V859A, R1008Q, A1341V, C1457S, G1909A, S1930Y, L2130I and V2417A according to SEQ ID NO: 25, wherein said nucleic acid molecule encodes an amino acid sequence derived from genotype 1a and said adaptive mutations of group a).

20. The isolated nucleic acid molecule according to claim 1, which encodes for strain TNcc-HI-18A (SEQ ID NO: 2), strain TNcc-HI-18B (SEQ ID NO: 3), strain TNcc-HI (SEQ ID NO: 4), strain J6cc-HI (SEQ ID NO: 5) or strain DBNcc-HI (SEQ ID NO: 6).

21. The nucleic acid molecule according to claim 1, comprised in a composition, wherein the nucleic acid molecule is suspended in a suitable amount of a pharmaceutical acceptable diluent or excipient.

22. The nucleic acid molecule according to claim 1, comprised in a cell.

23. A method for producing a hepatitis C virus particle or a hepatitis C virus-like particle comprising culturing a cell or raising an animal to to produce either the virus or the hepatitis C virus-like particle, wherein the cell or animal comprises a nucleic acid molecule encoding a human hepatitis C virus or the hepatitis C virus like particle selected from:
a hepatitis C virus derived from genotype 1a and said nucleic acid molecule encodes an amino acid sequence comprising one of the following groups of adaptive mutations:
a) S2482G, N2651H, 12902L and H2986R according to SEQ ID NO: 25; or
b) T2357A, S2375G, C2419R, M2834L and H2986R according to SEQ ID NO: 25;
a hepatitis C virus derived from genotype 2a and said nucleic acid molecule encodes an amino acid sequence comprising the following adaptive mutations: K78E, V140L, T329S, H434N, A575T, A760T, V773A, V828A, A1146V, I1330V, L1500P, V1835I, T2000A, L2363P, A2367P, E2783Q and L3013S according to SEQ ID NO: 26; or
a hepatitis C virus derived from genotype 3a and said nucleic acid molecule encodes an amino acid sequence comprising the following adaptive mutations: G395R, A856T, G875R, N1589S, 11753S, V2428A and D2807G according to SEQ ID NO: 27.

24. The method according to claim 23, wherein said nucleic acid molecule encodes an amino acid sequence further comprising the following adaptive mutations: T11N, G32S, V365A, F403L, N410K, V719I, V787A, V859A, R1008Q, A1341V, C1457S, G1909A, L2130I and V2417A according to SEQ ID NO: 25, wherein said nucleic acid molecule encodes an amino acid sequence derived from genotype 1a and said adaptive mutations of group a).

25. The method according to claim 23, wherein said nucleic acid molecule encodes an amino acid sequence further comprising the following adaptive mutations: T11N, G32S, L179P, V365A, F403L, N410K, V719I, V787A, V859A, R1008Q, A1341V, C1457S, G1909A, S1930Y, L2130I and V2417A according to SEQ ID NO: 25, wherein said nucleic acid molecule encodes an amino acid sequence derived from genotype 1a and said adaptive mutations of group a).

26. The method according to claim 23, wherein the cell or animal comprises a nucleic acid molecule comprising at least two genotypes selected from the group consisting of genotype 1a, genotype 2a and genotype 3a, thereby providing a mixture of hepatitis C virus particles.

27. The nucleic acid molecule according to claim 1 or the encoded amino acid sequence thereof comprised in a hepatitis C virus vaccine.

28. A method of treating, alleviating or preventing a hepatitis C virus infection, the method comprising administering to a subject a hepatitis C virus particle or a hepatitis C virus like particle comprising a nucleic acid sequence or a hepatitis C virus vaccine comprising the nucleic acid sequence or the encoded amino acid sequence thereof, wherein the nucleic acid molecule encoding the human hepatitis C virus or the hepatitis C virus like particle is selected from:
a hepatitis C virus derived from genotype 1a and said nucleic acid molecule encodes an amino acid sequence comprising one of the following groups of adaptive mutations:
a) S2482G, N2651H, 12902L and H2986R according to SEQ ID NO: 25; or
b) T2357A, S2375G, C2419R, M2834L and H2986R according to SEQ ID NO: 25;
a hepatitis C virus derived from genotype 2a and said nucleic acid molecule encodes an amino acid sequence comprising the following adaptive mutations: K78E, V140L, T329S, H434N, A575T, A760T, V773A, V828A, A1146V, I1330V, L1500P, V1835I, T2000A, L2363P, A2367P, E2783Q and L3013S according to SEQ ID NO: 26; or
a hepatitis C virus derived from genotype 3a and said nucleic acid molecule encodes an amino acid sequence comprising the following adaptive mutations: G395R, A856T, G875R, N1589S, 11753S, V2428A and D2807G according to SEQ ID NO: 27.

29. The method according to claim 28, wherein said nucleic acid molecule encodes an amino acid sequence further comprising the following adaptive mutations: T11N, G32S, V365A, F403L, N410K, V719I, V787A, V859A, R1008Q, A1341V, C1457S, G1909A, L2130I and V2417A according to SEQ ID NO: 25, wherein said nucleic acid molecule encodes an amino acid sequence derived from genotype 1a and said adaptive mutations of group a).

30. The method according to claim 28, wherein said nucleic acid molecule encodes an amino acid sequence further comprising the following adaptive mutations: T11N, G32S, L179P, V365A, F403L, N410K, V719I, V787A, V859A, R1008Q, A1341V, C1457S, G1909A, S1930Y, L2130I and V2417A according to SEQ ID NO: 25, wherein said nucleic acid molecule encodes an amino acid sequence derived from genotype 1a and said adaptive mutations of group a).

31. A method for screening an anti-hepatitis C virus substance, comprising
a) culturing a cell comprising a nucleic acid molecule, which replicates human hepatitis C virus or a hepatitis C virus like particle; and
b) detecting the replicating RNA or the virus particles in the resulting culture
wherein the nucleic acid molecule encodes a human hepatitis C virus or a hepatitis C virus like particle selected from:
a hepatitis C virus derived from genotype 1a and said nucleic acid molecule encodes an amino acid sequence comprising one of the following groups of adaptive mutations:
a) S2482G, N2651H, 12902L and H2986R according to SEQ ID NO: 25; or
b) T2357A, S2375G, C2419R, M2834L and H2986R according to SEQ ID NO: 25;
a hepatitis C virus derived from genotype 2a and said nucleic acid molecule encodes an amino acid sequence comprising the following adaptive mutations: K78E, V140L, T329S, H434N, A575T, A760T, V773A, V828A, A1146V, I1330V, L1500P, V1835I, T2000A, L2363P, A2367P, E2783Q and L3013S according to SEQ ID NO: 26; or
a hepatitis C virus derived from genotype 3a and said nucleic acid molecule encodes an amino acid sequence comprising the following adaptive mutations: G395R, A856T, G875R, N1589S, 11753S, V2428A and D2807G according to SEQ ID NO: 27.

32. The method according to claim 31, wherein said nucleic acid molecule encodes an amino acid sequence further comprising the following adaptive mutations: T11N, G32S, V365A, F403L, N410K, V719I, V787A, V859A, R1008Q, A1341V, C1457S, G1909A, L2130I and V2417A according to SEQ ID NO: 25, wherein said nucleic acid molecule encodes an amino acid sequence derived from genotype 1a and said adaptive mutations of group a).

33. The method according to claim 31, wherein said nucleic acid molecule encodes an amino acid sequence further comprising the following adaptive mutations: T11N, G32S, L179P, V365A, F403L, N410K, V719I, V787A, V859A, R1008Q, A1341V, C1457S, G1909A, S1930Y, L2130I and V2417A according to SEQ ID NO: 25, wherein said nucleic acid molecule encodes an amino acid sequence derived from genotype 1a and said adaptive mutations of group a).
